SEQUENCE LISTING (238 sequences)

SEQ ID NO: 1 - protein, 19 aa, Artificial sequence
  Synthetic polypeptide
RCRERRRNERLRRESVRPV

SEQ ID NO: 2 - protein, 29 aa, Artificial sequence
  Synthetic polypeptide
RKGRMMDVKKCGIQDTNSKKQSDTHLEET

SEQ ID NO: 3 - protein, 32 aa, Artificial sequence
  Synthetic polypeptide
KKRHMASYSMCSDPSTRDPPGRPEPYVEVYLI

SEQ ID NO: 4 - protein, 35 aa, Artificial sequence
  Synthetic polypeptide
TKKKYSSSVHDPNGEYMFMRAVNTAKKSRLTDVTL

SEQ ID NO: 5 - protein, 35 aa, Artificial sequence
  Synthetic polypeptide
MEESVVRPSVFVVDGQTDIPFTRLGRSHRRQSCSV

SEQ ID NO: 6 - protein, 39 aa, Artificial sequence
  Synthetic polypeptide
SLSKMLKKRSPLTTGVYVKMPPTEPECEKQFQPYFIPIN

SEQ ID NO: 7 - protein, 40 aa, Artificial sequence
  Synthetic polypeptide
ARTQIKKLCSWRDKNSAACVVYEDMSHSRCNTLSSPNQYQ

SEQ ID NO: 8 - protein, 42 aa, Artificial sequence
  Synthetic polypeptide
KRGRKKLLYIFKQPFMRPVQTTQEEDGCSCRFPEEEEGGCEL

SEQ ID NO: 9 - protein, 43 aa, Artificial sequence
  Synthetic polypeptide
WVRSKRSRLLHSDYMNMTPRRPGPTRKHYQPYAPPRDFAAYRS

SEQ ID NO: 10 - protein, 49 aa, Artificial sequence
  Synthetic polypeptide
HQRRKYRSNKGESPVEPAEPCHYSCPREEEGSTIPIQEDYRKPEPACSP

SEQ ID NO: 11 - protein, 51 aa, Artificial sequence
  Synthetic polypeptide
RRQWRPRRFSALEQGIHPPQAQSKIEELEQEPEPEPEPEPEPEPEPEPEQL

SEQ ID NO: 12 - protein, 54 aa, Artificial sequence
  Synthetic polypeptide
HIWQLRSQCMWPRETQLLLEVPPSTEDARSCQFPEEERGERSAEEKGRLGDLWV

SEQ ID NO: 13 - protein, 61 aa, Artificial sequence
  Synthetic polypeptide
NRRRRRERRDLFTESWDTQKAPNNYRSPISTSQPTNQSMDDTREDIYVNYPTFSRRPKTRV

SEQ ID NO: 14 - protein, 62 aa, Artificial sequence
  Synthetic polypeptide
KKVAKKPTNKAPHPKQEPQEINFPDDLPGSNTAAPVQETLHGCQPVTQEDGKESRISVQERQ

SEQ ID NO: 15 - protein, 77 aa, Artificial sequence
  Synthetic polypeptide
KWYSHSKEKIQNLSLISLANLPPSGLANAVAEGIRSEENIYTIEENVYEVEEPNEYYCYVSSRQQPSQPLGCRFAMP

SEQ ID NO: 16 - protein, 80 aa, Artificial sequence
  Synthetic polypeptide
RKKKALRIHSVEGDLRRKSAGQEEWSPSAPSPPGSCVQAEAAPAGLCGEQRGEDCAELHDYFNVLSYRSLGNCSFFTETG

SEQ ID NO: 17 - protein, 83 aa, Artificial sequence
  Synthetic polypeptide
KLRKAHVIWKKENEVSEHTLESYRSRSNNEETSSEEKNGQSSHPMRCMNYITKLYSEAKTKRKENVQHSKLEEKHIQVPESIV

SEQ ID NO: 18 - protein, 98 aa, Artificial sequence
  Synthetic polypeptide
ICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 19 - protein, 101 aa, Artificial sequence
  Synthetic polypeptide
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 20 - protein, 107 aa, Artificial sequence
  Synthetic polypeptide
CCRKKRREEKYEKEVHHDIREDVPPPKSRTSTARSYIGSNHSSLGSMSPSNMEGYSKTQYNQVPSEDFERTPQSPTLPPAKVAAPNLSRMGAIPVMIPAQSKDGSIV

SEQ ID NO: 21 - protein, 114 aa, Artificial sequence
  Synthetic polypeptide
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 22 - protein, 117 aa, Artificial sequence
  Synthetic polypeptide
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 23 - protein, 120 aa, Artificial sequence
  Synthetic polypeptide
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 24 - protein, 187 aa, Artificial sequence
  Synthetic polypeptide
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 25 - protein, 108 aa, Artificial sequence
  Synthetic polypeptide
TYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQ

SEQ ID NO: 26 - protein, 42 aa, Artificial sequence
  Synthetic polypeptide
ALYLLRRDQRLPPDAHKPPGGGSFRTPIQEEQADAHSTLAKI

SEQ ID NO: 27 - protein, 51 aa, Artificial sequence
  Synthetic polypeptide
MGWIRGRRSRHSWEMSEFHNYNLDLKKSDFSTRWQKQRCPVVKSKCRENAS

SEQ ID NO: 28 - protein, 48 aa, Artificial sequence
  Synthetic polypeptide
KKYFFKKEVQQLSVSFSSLQIKALQNAVEKEVQAEDNIYIENSLYATD

SEQ ID NO: 29 - protein, 22 aa, Artificial sequence
  Synthetic polypeptide
SGFLQEKVWVMLVTSLVALQAL

SEQ ID NO: 30 - protein, 85 aa, Artificial sequence
  Synthetic polypeptide
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 31 - protein, 111 aa, Artificial sequence
  Synthetic polypeptide
ACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 32 - protein, 95 aa, Artificial sequence
  Synthetic polypeptide
MAEAITYADLRFVKAPLKKSISSRLGQDPGADDDGEITYENVQVPAVLGVPSSLASSVLGDKAAVKSEQPTASWRAVTSPAVGRILPCRTTCLRY

SEQ ID NO: 33 - protein, 141 aa, Artificial sequence
  Synthetic polypeptide
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 34 - protein, 45 aa, Artificial sequence
  Synthetic polypeptide
RKWCQYQKEIMERPPPFKPPPPPIKYTCIQEPNESDLPYHEMETL

SEQ ID NO: 35 - protein, 84 aa, Artificial sequence
  Synthetic polypeptide
LRKRRDSLSLSTQRTQGPAESARNLEYVSVSPTNNTVYASVTHSNRETEIWTPRENDTITIYSTINHSKESKPTFSRATALDNV

SEQ ID NO: 36 - protein, 88 aa, Artificial sequence
  Synthetic polypeptide
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 37 - protein, 82 aa, Artificial sequence
  Synthetic polypeptide
KTHRRKAARTAVGRNDTHPTTGSASPKHQKKSKLHGPTETSSCSGAAPTVEMDEELHYASLNFHGMNPSKDTSTEYSEVRTQ

SEQ ID NO: 38 - protein, 38 aa, Artificial sequence
  Synthetic polypeptide
MTDSVIYSMLELPTATQAQNDYGPQQKSSSSRPSCSCL

SEQ ID NO: 39 - protein, 45 aa, Artificial sequence
  Synthetic polypeptide
MDQQAIYAELNLPTDSGPESSSPSSLPRDVCQGSPWHQFALKLSC

SEQ ID NO: 40 - protein, 168 aa, Artificial sequence
  Synthetic polypeptide
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 41 - protein, 83 aa, Artificial sequence
  Synthetic polypeptide
RWCSNKKNAAVMDQESAGNRTANSEDSDEQDPQEVTYTQLNHCVFTQRKITRPSQRPKTPPTDIIVYTELPNAESRSKVVSCP

SEQ ID NO: 42 - protein, 84 aa, Artificial sequence
  Synthetic polypeptide
HLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 43 - protein, 10 aa, Artificial sequence
  Synthetic polypeptide
MAVFKTTLWR

SEQ ID NO: 44 - protein, 29 aa, Artificial sequence
  Synthetic polypeptide
KGSQRVPEEPGEQPIYMNFSEPLTKDMAT

SEQ ID NO: 45 - protein, 11 aa, Artificial sequence
  Synthetic polypeptide
VNRPQWAPPGR

SEQ ID NO: 46 - protein, 8 aa, Artificial sequence
  Synthetic polypeptide
VTLRSFVP

SEQ ID NO: 47 - protein, 35 aa, Artificial sequence
  Synthetic polypeptide
AAWHGQKPGTHPPSELDCGHDPGYQLQTLPGLRDT

SEQ ID NO: 48 - protein, 32 aa, Artificial sequence
  Synthetic polypeptide
QHSQRSPPRCSQEANSRKDNAPFRVVEPWEQI

SEQ ID NO: 49 - protein, 168 aa, Artificial sequence
  Synthetic polypeptide
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 50 - protein, 116 aa, Artificial sequence
  Synthetic polypeptide
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 51 - protein, 128 aa, Artificial sequence
  Synthetic polypeptide
MAKRKQGNRLGVCGRFLSSRVSGMNPSSVVHHVSDSGPAAELPLDVPHIRLDSPPSFDNTTYTSLPLDSPSGKPSLPAPSSLPPLPPKVLVCSKPVTYATVIFPGGNKGGGTSCGPAQNPPNNQTPSS

SEQ ID NO: 52 - protein, 61 aa, Artificial sequence
  Synthetic polypeptide
KVNGCRKYKLNKTESTPVVEEDEMQPYASYTEKNNPLYDTTNKVKASEALQSEVDTDLHTL

SEQ ID NO: 53 - protein, 98 aa, Artificial sequence
  Synthetic polypeptide
RMFQKWIKAGDHSELSQNPKQAATQSELHYANLELLMWPLQEKPAPPREVEVEYSTVASPREELHYASVVFDSNTNRIAAQRPREEEPDSDYSVIRKT

SEQ ID NO: 54 - protein, 113 aa, Artificial sequence
  Synthetic polypeptide
WRMMKYQQKAAGMSPEQVLQPLEGDLCYADLTLQLAGTSPQKATTKLSSAQVDQVEVEYVTMASLPKEDISYASLTLGAEDQEPTYCNMGHLSSHLPGRGPEEPTEYSTISRP

SEQ ID NO: 55 - protein, 37 aa, Artificial sequence
  Synthetic polypeptide
MSDSKEPRLQQLGLLEEEQLRGLGFRQTRGYKSLAGC

SEQ ID NO: 56 - protein, 29 aa, Artificial sequence
  Synthetic polypeptide
RKSSGGKGGSYSQAACSDSAQGSDVSLTA

SEQ ID NO: 57 - protein, 95 aa, Artificial sequence
  Synthetic polypeptide
LPKYKTRKAMRNNVPRDRGDTAMEVGIYANILEKQAKEESVPEVGSRPCVSTAQDEAKHSQELQYATPVFQEVAPREQEACDSYKSGYVYSELNF

SEQ ID NO: 58 - protein, 178 aa, Artificial sequence
  Synthetic polypeptide
RRRHRGKFRKDVQKEKDLQLSSGAEEPITRKGELQKRPNPAAATQEESLYASVEDMQTEDGVELNSWTPPEEDPQGETYAQVKPSRLRKAGHVSPSVMSREQLNTEYEQAEEGQGANNQAAESGESQDVTYAQLCSRTLRQGAAASPLSQAGEAPEEPSVYATLAAARPEAVPKDMEQ

SEQ ID NO: 59 - protein, 42 aa, Artificial sequence
  Synthetic polypeptide
SAGSAGSAGSAGSAGSAGSAGSAGSAGSAGSAGSAGSAGSAG

SEQ ID NO: 60 - protein, 93 aa, Artificial sequence
  Synthetic polypeptide
KKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 61 - protein, 118 aa, Artificial sequence
  Synthetic polypeptide
KCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 62 - protein, 74 aa, Artificial sequence
  Synthetic polypeptide
HFGKTGRASDQRDLTEHKPSVSNHTQDHSNDPPNKMNEVTYSTLNFEAQQPTQPTSASPSLTATEIIYSEVKKQ

SEQ ID NO: 63 - protein, 112 aa, Artificial sequence
  Synthetic polypeptide
DVVCTGWLRKSPPEKKLRRYAWKKRWFILRSGRMSGDPDVLEYYKNDHSKKPLRIINLNFCEQVDAGLTFNKKELQDSFVFDIKTSERTFYLVAETEEDMNKWVQSICQICG

SEQ ID NO: 64 - protein, 116 aa, Artificial sequence
  Synthetic polypeptide
AVMEGPLFLQSQRFGTKRWRKTWAVLYPASPHGVARLEFFDHKGSSSGGGRGSSRRLDCKVIRLAECVSVAPVTVETPPEPGATAFRLDTAQRSHLLAADAPSSAAWVQTLCRNAF

SEQ ID NO: 65 - protein, 111 aa, Artificial sequence
  Synthetic polypeptide
GAVKQGFLYLQQQQTFGKKWRRFGASLYGGSDCALARLELQEGPEKPRRCEAARKVIRLSDCLRVAEAGGEASSPRDTSAFFLETKERLYLLAAPAAERGDWVQAICLLAF

SEQ ID NO: 66 - protein, 44 aa, Artificial sequence
  Synthetic polypeptide
FWVRSKRSRLLHSDYMNMTPRRPGPTRKHYQPYAPPRDFAAYRS

SEQ ID NO: 67 - protein, 37 aa, Artificial sequence
  Synthetic polypeptide
RRDQRLPPDAHKPPGGGSFRTPIQEEQADAHSTLAKI

SEQ ID NO: 68 - protein, 49 aa, Artificial sequence
  Synthetic polypeptide
HQRRKYRSNKGESPVEPAEPCRYSCPREEEGSTIPIQEDYRKPEPACSP

SEQ ID NO: 69 - protein, 60 aa, Artificial sequence
  Synthetic polypeptide
CVKRRKPRGDVVKVIVSVQRKRQEAEGEATVIEALQAPPDVTTVAVEETIPSFTGRSPNH

SEQ ID NO: 70 - protein, 4 aa, Artificial sequence
  Synthetic polypeptide
  MISC_FEATURE (2)..(2): Xaa may be any amino acid.
  MISC_FEATURE (3)..(3): Xaa may be any amino acid.
  MISC_FEATURE (4)..(4): Xaa may be Leu or Ile.
YXXX

SEQ ID NO: 71 - protein, 16 aa, Artificial sequence
  Synthetic polypeptide
  MISC_FEATURE (2)..(2): Xaa may be any amino acid.
  MISC_FEATURE (3)..(3): Xaa may be any amino acid.
  MISC_FEATURE (4)..(4): Xaa may be Leu or Ile.
  MISC_FEATURE (5)..(12): Xaa may be any amino acid.
  MISC_FEATURE (11)..(12): These two residues may be missing.
  MISC_FEATURE (14)..(14): Xaa may be any amino acid.
  MISC_FEATURE (15)..(15): Xaa may be any amino acid.
  MISC_FEATURE (16)..(16): Xaa may be Leu or Ile.
YXXXXXXXXXXXYXXX

SEQ ID NO: 72 - protein, 113 aa, Artificial sequence
  Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 73 - protein, 112 aa, Artificial sequence
  Synthetic polypeptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 74 - protein, 102 aa, Artificial sequence
  Synthetic polypeptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 75 - protein, 101 aa, Artificial sequence
  Synthetic polypeptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 76 - protein, 21 aa, Artificial sequence
  Synthetic polypeptide
ESPYQELQGQRSDVYSDLNTQ

SEQ ID NO: 77 - protein, 86 aa, Artificial sequence
  Synthetic polypeptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 78 - protein, 21 aa, Artificial sequence
  Synthetic polypeptide
DGVYTGLSTRNQETYETLKHE

SEQ ID NO: 79 - protein, 171 aa, Artificial sequence
  Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 80 - protein, 127 aa, Artificial sequence
  Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 81 - protein, 21 aa, Artificial sequence
  Synthetic polypeptide
DQVYQPLRDRDDAQYSHLGGN

SEQ ID NO: 82 - protein, 207 aa, Artificial sequence
  Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 83 - protein, 21 aa, Artificial sequence
  Synthetic polypeptide
NPDYEPIRKGQRDLYSGLNQR

SEQ ID NO: 84 - protein, 182 aa, Artificial sequence
  Synthetic polypeptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 85 - protein, 21 aa, Artificial sequence
  Synthetic polypeptide
DQLYQPLKDREDDQYSHLQGN

SEQ ID NO: 86 - protein, 163 aa, Artificial sequence
  Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 164 aa, Artificial sequence
  Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 88 - protein, 112 aa, Artificial sequence
  Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 21 aa, Artificial sequence
  Synthetic polypeptide
NQLYNELNLGRREEYDVLDKR

SEQ ID NO: 90 - protein, 22 aa, Artificial sequence
  Synthetic polypeptide
EGLYNELQKDKMAEAYSEIGMK

SEQ ID NO: 91 - protein, 21 aa, Artificial sequence
  Synthetic polypeptide
DGLYQGLSTATKDTYDALHMQ

SEQ ID NO: 92 - protein, 226 aa, Artificial sequence
  Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 93 - protein, 188 aa, Artificial sequence
  Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 94 - protein, 21 aa, Artificial sequence
  Synthetic polypeptide
ENLYEGLNLDDCSMYEDISRG

SEQ ID NO: 95 - protein, 20 aa, Artificial sequence
  Synthetic polypeptide
RPRRSPAQDGKVYINMPGRG

SEQ ID NO: 96 - protein, 68 aa, Artificial sequence
  Synthetic polypeptide
FWVLVVVGGVLACYSLLVTVAFIIFWVRSKRSRLLHSDYMNMTPRRPGPTRKHYQPYAPPRDFAAYRS

SEQ ID NO: 97 - protein, 619 aa, Artificial sequence
  Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 98 - protein, 24 aa, Artificial sequence
  Synthetic polypeptide
IYIWAPLAGTCGVLLLSLVITLYC

SEQ ID NO: 99 - protein, 23 aa, Artificial sequence
  Synthetic polypeptide
LGLLVAGVLVLLVSLGVAIHLCC

SEQ ID NO: 100 - protein, 25 aa, Artificial sequence
  Synthetic polypeptide
ALIVLGGVAGLLLFIGLGIFFCVRC

SEQ ID NO: 101 - protein, 23 aa, Artificial sequence
  Synthetic polypeptide
LCYLLDGILFIYGVILTALFLRV

SEQ ID NO: 102 - protein, 26 aa, Artificial sequence
  Synthetic polypeptide
WVLVVVGGVLACYSLLVTVAFIIFWV

SEQ ID NO: 103 - protein, 26 aa, Artificial sequence
  Synthetic polypeptide
VAAILGLGLVLGLLGPLAILLALYLL

SEQ ID NO: 104 - protein, 24 aa, Artificial sequence
  Synthetic polypeptide
ALPAALAVISFLLGLGLGVACVLA

SEQ ID NO: 105 - protein, 5 aa, Artificial sequence
  Synthetic polypeptide
  MISC_FEATURE (5)..(5): This residue may be repeated at least once.
GSGGS

SEQ ID NO: 106 - protein, 4 aa, Artificial sequence
  Synthetic polypeptide
  MISC_FEATURE (4)..(4): This residue may be repeated at least once.
GGGS

SEQ ID NO: 107 - protein, 4 aa, Artificial sequence
  Synthetic polypeptide
GGSG

SEQ ID NO: 108 - protein, 5 aa, Artificial sequence
  Synthetic polypeptide
GGSGG

SEQ ID NO: 109 - protein, 5 aa, Artificial sequence
  Synthetic polypeptide
GSGSG

SEQ ID NO: 110 - protein, 5 aa, Artificial sequence
  Synthetic polypeptide
GSGGG

SEQ ID NO: 111 - protein, 5 aa, Artificial sequence
  Synthetic polypeptide
GGGSG

SEQ ID NO: 112 - protein, 5 aa, Artificial sequence
  Synthetic polypeptide
GSSSG

SEQ ID NO: 113 - protein, 5 aa, Artificial sequence
  Synthetic polypeptide
  MISC_FEATURE (1)..(5): This stretch of residues may be repeated at least once.
GSGGS

SEQ ID NO: 114 - protein, 4 aa, Artificial sequence
  Synthetic polypeptide
  MISC_FEATURE (1)..(4): This stretch of residues may be repeated at least once.
GGGS

SEQ ID NO: 115 - protein, 4 aa, Artificial sequence
  Synthetic polypeptide
CPPC

SEQ ID NO: 116 - protein, 5 aa, Artificial sequence
  Synthetic polypeptide
DKTHT

SEQ ID NO: 117 - protein, 15 aa, Artificial sequence
  Synthetic polypeptide
CPEPKSCDTPPPCPR

SEQ ID NO: 118 - protein, 12 aa, Artificial sequence
  Synthetic polypeptide
ELKTPLGDTTHT

SEQ ID NO: 119 - protein, 10 aa, Artificial sequence
  Synthetic polypeptide
KSCDKTHTCP

SEQ ID NO: 120 - protein, 7 aa, Artificial sequence
  Synthetic polypeptide
KCCVDCP

SEQ ID NO: 121 - protein, 7 aa, Artificial sequence
  Synthetic polypeptide
KYGPPCP

SEQ ID NO: 122 - protein, 15 aa, Artificial sequence
  Synthetic polypeptide
EPKSCDKTHTCPPCP

SEQ ID NO: 123 - protein, 12 aa, Artificial sequence
  Synthetic polypeptide
ERKCCVECPPCP

SEQ ID NO: 124 - protein, 17 aa, Artificial sequence
  Synthetic polypeptide
ELKTPLGDTTHTCPRCP

SEQ ID NO: 125 - protein, 12 aa, Artificial sequence
  Synthetic polypeptide
SPNMVPHAHHAQ

SEQ ID NO: 126 - protein, 15 aa, Artificial sequence
  Synthetic polypeptide
EPKSCDKTYTCPPCP

SEQ ID NO: 127 - protein, 45 aa, Artificial sequence
  Synthetic polypeptide
TTTPAPRPPTPAPTIASQPLSLRPEACRPAAGGAVHTRGLDFACD

SEQ ID NO: 128 - protein, 9 aa, Artificial sequence
  Synthetic polypeptide
SLLMWITQC

SEQ ID NO: 129 - protein, 275 aa, Artificial sequence
  Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 130 - protein, 275 aa, Artificial sequence
  Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 131 - protein, 309 aa, Artificial sequence
  Synthetic polypeptide
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 132 - protein, 309 aa, Artificial sequence
  Synthetic polypeptide
MSIGLLCCAALSLLWAGPVNAGVTQTPKFQVLKTGQSMTLQCAQDMNHEYMSWYRQDPGMGLRLIHYSVAAGITDQGEVPNGYNVSRSTTEDFPLRLLSAAPSQTSVYFCASSYVGNTGELFFGEGSRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 133 - protein, 208 aa, Artificial sequence
  Synthetic polypeptide
MQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 134 - protein, 242 aa, Artificial sequence
  Synthetic polypeptide
MGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRAD

SEQ ID NO: 135 - protein, 525 aa, Artificial sequence
  Synthetic polypeptide
MDFQVQIFSFLLISASVIMSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNPGGGGGSGGGGSGGGGSGGGGSGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSTSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 136 - protein, 549 aa, Artificial sequence
  Synthetic polypeptide
MDFQVQIFSFLLISASVIELGGGGSGSMDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGGGGSGGGGSGGGGSGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSTSGGGGSGGGGSGGGGSGGGGSDIMSRGSDLGKKLLEAARAGQDDEVRILMANGADVNAKDEYGLTPLYLATAHGHLEIVEVLLKNGADVNAVDAIGFTPLHLAAFIGHLEIAEVLLKHGADVNAQDKFGKTAFDISIGNGNEDLAEILQKLNEQKLISEEDLNVDGGGGSGGGGSGGGGSGGGGSLESGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 137 - protein, 258 aa, Artificial sequence
  Synthetic polypeptide
YDLLQRLGGGTYGEVFKARDKVSGDLVALKMVKMEPDDDVSTLQKEILILKTCRHANIVAYHGSYLWLQKLWICMEFCGAGSLQDIYQVTGSLSELQISYVCREVLQGLAYLHSQKKIHRDIKGANILINDAGEVRLADFGISAQIGATLARRLSFIGTPYWMAPEVAAVALKGGYNELCDIWSLGITAIELAELQPPLFDVHPLRVLFLMTKSGYQPPRLKEKGKWSAAFHNFIKVTLTKSPKKRPSATKMLSHQLV

SEQ ID NO: 138 - protein, 272 aa, Artificial sequence
  Synthetic polypeptide
FWEEFESLQKQEVKNLHQRLEGQRPENKGKNRYKNILPFDHSRVILQGRDSNIPGSDYINANYIKNQLLGPDENAKTYIASQGCLEATVNDFWQMAWQENSRVIVMTTREVEKGRNKCVPYWPEVGMQRAYGPYSVTNCGEHDTTEYKLRTLQVSPLDNGDLIREIWHYQYLSWPDHGVPSEPGGVLSFLDQINQRQESLPHAGPIIVHCSAGIGRTGTIIVIDMLMENISTKGLDCDIDIQKTIQMVRAQRSGMVQTEAQYKFIYVAIAQF

SEQ ID NO: 139 - protein, 275 aa, Artificial sequence
  Synthetic polypeptide
FWEEFETLQQQECKLLYSRKEGQRQENKNKNRYKNILPFDHTRVVLHDGDPNEPVSDYINANIIMPEFETKCNNSKPKKSYIATQGCLQNTVNDFWRMVFQENSRVIVMTTKEVERGKSKCVKYWPDEYALKEYGVMRVRNVKESAAHDYTLRELKLSKVGQALLQGNTERTVWQYHFRTWPDHGVPSDPGGVLDFLEEVHHKQESIMDAGPVVVHCSAGIGRTGTFIVIDILIDIIREKGVDCDIDVPKTIQMVRSQRSGMVQTEAQYRFIYMA

SEQ ID NO: 140 - protein, 270 aa, Artificial sequence
  Synthetic polypeptide
GPQKRCLFVCRHGERMDVVFGKYWLSQCFDAKGRYIRTNLNMPHSLPQRSGGFRDYEKDAPITVFGCMQARLVGEALLESNTIIDHVYCSPSLRCVQTAHNILKGLQQENHLKIRVEPGLFEWTKWVAGSTLPAWIPPSELAAANLSVDTTYRPHIPISKLVVSESYDTYISRSFQVTKEIISECKSKGNNILIVAHASSLEACTCQLQGLSPQNSKDFVQMVRKIPYLGFCSCEELGETGIWQLTDPPILPLTHGPTGGFNWRETLLQE

SEQ ID NO: 141 - protein, 255 aa, Artificial sequence
  Synthetic polypeptide
LKLLQTIGKGEFGDVMLGDYRGNKVAVKCIKNDATAQAFLAEASVMTQLRHSNLVQLLGVIVEEKGGLYIVTEYMAKGSLVDYLRSRGRSVLGGDCLLKFSLDVCEAMEYLEGNNFVHRDLAARNVLVSEDNVAKVSDFGLTKEASSTQDTGKLPVKWTAPEALREKKFSTKSDVWSFGILLWEIYSFGRVPYPRIPLKDVVPRVEKGYKMDAPDGCPPAVYEVMKNCWHLDAAMRPSFLQLREQLEHIKTHELH

SEQ ID NO: 142 - protein, 9 aa, Artificial sequence
  Synthetic polypeptide
YPYDVPDYA

SEQ ID NO: 143 - protein, 8 aa, Artificial sequence
  Synthetic polypeptide
DYKDDDDK

SEQ ID NO: 144 - protein, 10 aa, Artificial sequence
  Synthetic polypeptide
EQKLISEEDL

SEQ ID NO: 145 - protein, 5 aa, Artificial sequence
  Synthetic polypeptide
HHHHH

SEQ ID NO: 146 - protein, 6 aa, Artificial sequence
  Synthetic polypeptide
HHHHHH

SEQ ID NO: 147 - protein, 8 aa, Artificial sequence
  Synthetic polypeptide
WSHPQFEK

SEQ ID NO: 148 - protein, 5 aa, Artificial sequence
  Synthetic polypeptide
RYIRS

SEQ ID NO: 149 - protein, 4 aa, Artificial sequence
  Synthetic polypeptide
FHHT

SEQ ID NO: 150 - protein, 17 aa, Artificial sequence
  Synthetic polypeptide
WEAAAREACCRECCARA

SEQ ID NO: 151 - protein, 16 aa, Artificial Sequence
  Synthetic polypeptide
DAFQLRQLILRGLQDD

SEQ ID NO: 152 - protein, 16 aa, Artificial sequence
  Synthetic polypeptide
DAFQLRQLILRGLQDD

SEQ ID NO: 153 - protein, 13 aa, Artificial sequence
  Synthetic polypeptide
SPGSREWFKDMLS

SEQ ID NO: 154 - protein, 13 aa, Artificial sequence
  Synthetic polypeptide
SPGSREWFKDMLS

SEQ ID NO: 155 - protein, 25 aa, Artificial Sequence
  Synthetic polypeptide
CPSSHSSLTERHKILHRLLQEGSPS

SEQ ID NO: 156 - protein, 21 aa, Artificial sequence
  Synthetic polypeptide
SLTARHKILHRLLQEGSPSDI

SEQ ID NO: 157 - protein, 19 aa, Artificial Sequence
  Synthetic polypeptide
ESKGHKKLLQLLTCSSDDR

SEQ ID NO: 158 - protein, 21 aa, Artificial sequence
  Synthetic polypeptide
PKKENNALLRYLLDRDDPSDV

SEQ ID NO: 159 - protein, 17 aa, Artificial sequence
  Synthetic polypeptide
AEEPSLLKKLLLAPANT

SEQ ID NO: 160 - protein, 21 aa, Artificial sequence
  Synthetic polypeptide
QEAEEPSLLKKLLLAPANTQL

SEQ ID NO: 161 - protein, 21 aa, Artificial sequence
  Synthetic polypeptide
SKVSQNPILTSLLQITGNGGS

SEQ ID NO: 162 - protein, 25 aa, Artificial sequence
  Synthetic polypeptide
GHSFADPASNLGLEDIIRKALMGSF

SEQ ID NO: 163 - protein, 17 aa, Artificial sequence
  Synthetic polypeptide
PRQGSILYSMLTSAKQT

SEQ ID NO: 164 - protein, 17 aa, Artificial sequence
  Synthetic polypeptide
AANNSLLLHLLKSQTIP

SEQ ID NO: 165 - protein, 21 aa, Artificial sequence
  Synthetic polypeptide
PKKKENALLRYLLDKDDTKDI

SEQ ID NO: 166 - protein, 16 aa, Artificial sequence
  Synthetic polypeptide
DAFQLRQLILRGLQDD

SEQ ID NO: 167 - protein, 13 aa, Artificial sequence
  Synthetic polypeptide
SPGSREWFKDMLS

SEQ ID NO: 168 - protein, 21 aa, Artificial sequence
  Synthetic polypeptide
GNTKNHPMLMNLLKDNPAQDF

SEQ ID NO: 169 - protein, 16 aa, Artificial sequence
  Synthetic polypeptide
SSKGVLWRMLAEPVSR

SEQ ID NO: 170 - protein, 15 aa, Artificial sequence
  Synthetic polypeptide
SRTLQLDWGTLYWSR

SEQ ID NO: 171 - protein, 15 aa, Artificial sequence
  Synthetic polypeptide
SSNHQSSRLIELLSR

SEQ ID NO: 172 - protein, 19 aa, Artificial sequence
  Synthetic polypeptide
LKEKHKILHRLLQDSSSPV

SEQ ID NO: 173 - protein, 14 aa, Artificial sequence
  Synthetic polypeptide
QAQQKSLLQQLLTE

SEQ ID NO: 174 - protein, 21 aa, Artificial sequence
  Synthetic polypeptide
KYSQTSHKLVQLLTTTAEQQL

SEQ ID NO: 175 - protein, 21 aa, Artificial sequence
  Synthetic polypeptide
SLTARHKILHRLLQEGSPSDI

SEQ ID NO: 176 - protein, 21 aa, Artificial sequence
  Synthetic polypeptide
KESKDHQLLRYLLDKDEKDLR

SEQ ID NO: 177 - protein, 15 aa, Artificial sequence
  Synthetic polypeptide
PQAQQKSLLQQLLTE

SEQ ID NO: 178 - protein, 15 aa, Artificial sequence
  Synthetic polypeptide
PQAQQKSLRQQLLTE

SEQ ID NO: 179 - protein, 21 aa, Artificial sequence
  Synthetic polypeptide
HDSKGQTKLLQLLTTKSDQME

SEQ ID NO: 180 - protein, 21 aa, Artificial sequence
  Synthetic polypeptide
SLKEKHKILHRLLQDSSSPVD

SEQ ID NO: 181 - protein, 21 aa, Artificial sequence
  Synthetic polypeptide
PKKKENALLRYLLDKDDTKDI

SEQ ID NO: 182 - protein, 21 aa, Artificial sequence
  Synthetic polypeptide
LESKGHKKLLQLLTCSSDDRG

SEQ ID NO: 183 - protein, 21 aa, Artificial sequence
  Synthetic polypeptide
LLQEKHRILHKLLQNGNSPAE

SEQ ID NO: 184 - protein, 21 aa, Artificial sequence
  Synthetic polypeptide
KKKENNALLRYLLDRDDPSDA

SEQ ID NO: 185 - protein, 21 aa, Artificial sequence
  Synthetic polypeptide
QEAEEPSLLKKLLLAPANTQL

SEQ ID NO: 186 - protein, 21 aa, Artificial sequence
  Synthetic polypeptide
PEVDELSLLQKLLLATSYPTS

SEQ ID NO: 187 - protein, 21 aa, Artificial sequence
  Synthetic polypeptide
VSPREGSSLHKLLTLSRTPPE

SEQ ID NO: 188 - protein, 21 aa, Artificial sequence
  Synthetic polypeptide
SKVSQNPILTSLLQITGNGGS

SEQ ID NO: 189 - protein, 21 aa, Artificial sequence
  Synthetic polypeptide
GNTKNHPMLMNLLKDNPAQDF

SEQ ID NO: 190 - protein, 21 aa, Artificial sequence
  Synthetic polypeptide
DVTLTSPLLVNLLQSDISAGH

SEQ ID NO: 191 - protein, 21 aa, Artificial sequence
  Synthetic polypeptide
AMREAPTSLSQLLDNSGAPNV

SEQ ID NO: 192 - protein, 21 aa, Artificial sequence
  Synthetic polypeptide
DAASKHKQLSELLRGGSGSSI

SEQ ID NO: 193 - protein, 21 aa, Artificial sequence
  Synthetic polypeptide
KRKLIQQQLVLLLHAHKCQRR

SEQ ID NO: 194 - protein, 21 aa, Artificial sequence
  Synthetic polypeptide
DAASKHKQLSELLRSGSSPNL

SEQ ID NO: 195 - protein, 21 aa, Artificial sequence
  Synthetic polypeptide
GHPPAIQSLINLLADNRYLTA

SEQ ID NO: 196 - protein, 21 aa, Artificial sequence
  Synthetic polypeptide
TLQQQAQQLYSLLGQFNCLTH

SEQ ID NO: 197 - protein, 21 aa, Artificial sequence
  Synthetic polypeptide
GSRETSEKFKLLFQSYNVNDW

SEQ ID NO: 198 - protein, 21 aa, Artificial sequence
  Synthetic polypeptide
NANYPRSILTSLLLNSSQSST

SEQ ID NO: 199 - protein, 21 aa, Artificial sequence
  Synthetic polypeptide
IPIEPDYKFSTLLMMLKDMHD

SEQ ID NO: 200 - protein, 21 aa, Artificial sequence
  Synthetic polypeptide
ATPPPSPLLSELLKKGSLLPT

SEQ ID NO: 201 - protein, 21 aa, Artificial sequence
  Synthetic polypeptide
VDGHERAMLKRLLRIDSKCLH

SEQ ID NO: 202 - protein, 21 aa, Artificial sequence
  Synthetic polypeptide
HEDLDKVKLIEYYLTKNKEGP

SEQ ID NO: 203 - protein, 21 aa, Artificial sequence
  Synthetic polypeptide
ESPEFCLGLQTLLSLKCCIDL

SEQ ID NO: 204 - protein, 21 aa, Artificial sequence
  Synthetic polypeptide
AATTQNPVLSKLLMADQDSPL

SEQ ID NO: 205 - protein, 30 aa, Artificial sequence
  Synthetic polypeptide
MGQVPRTHRLITLADHICQIITQDFARNQV

SEQ ID NO: 206 - protein, 14 aa, Artificial sequence
  Synthetic polypeptide
NLGLEDIIRKALMG

SEQ ID NO: 207 - protein, 40 aa, Artificial sequence
  Synthetic polypeptide
APGVKGHQRVVTLAQHISEVITQDTYRHHPQQLSAPLPAP

SEQ ID NO: 208 - protein, 14 aa, Artificial sequence
  Synthetic polypeptide
NMGLEAIIRKALMG

SEQ ID NO: 209 - protein, 21 aa, Artificial sequence
  Synthetic polypeptide
RLTKTNPILYYMLQKGGNSVA

SEQ ID NO: 210 - protein, 21 aa, Artificial sequence
  Synthetic polypeptide
QDSIVLTYLEGLLMHQAAGGS

SEQ ID NO: 211 - protein, 21 aa, Artificial sequence
  Synthetic polypeptide
KGKQDSTLLASLLQSFSSRLQ

SEQ ID NO: 212 - protein, 21 aa, Artificial sequence
  Synthetic polypeptide
CYGVASSHLKTLLKKSKVKDQ

SEQ ID NO: 213 - protein, 21 aa, Artificial sequence
  Synthetic polypeptide
KPSVACSQLALLLSSEAHLQQ

SEQ ID NO: 214 - protein, 21 aa, Artificial sequence
  Synthetic polypeptide
KQAANNSLLLHLLKSQTIPKP

SEQ ID NO: 215 - protein, 21 aa, Artificial sequence
  Synthetic polypeptide
NSHQKVTLLQLLLGHKNEENV

SEQ ID NO: 216 - protein, 21 aa, Artificial sequence
  Synthetic polypeptide
NLLERRTVLQLLLGNPTKGRV

SEQ ID NO: 217 - protein, 21 aa, Artificial sequence
  Synthetic polypeptide
FSFSKNGLLSRLLRQNQDSYL

SEQ ID NO: 218 - protein, 21 aa, Artificial sequence
  Synthetic polypeptide
RESKSFNVLKQLLLSENCVRD

SEQ ID NO: 219 - protein, 21 aa, Artificial sequence
  Synthetic polypeptide
ELNADDAILRELLDESQKVMV

SEQ ID NO: 220 - protein, 21 aa, Artificial sequence
  Synthetic polypeptide
YENLPPAALRKLLRAEPERYR

SEQ ID NO: 221 - protein, 21 aa, Artificial sequence
  Synthetic polypeptide
MAFAGDEVLVQLLSGDKAPEG

SEQ ID NO: 222 - protein, 21 aa, Artificial sequence
  Synthetic polypeptide
SCCYLCIRLEGLLAPTASPRP

SEQ ID NO: 223 - protein, 21 aa, Artificial sequence
  Synthetic polypeptide
PSNKSVDVLAGLLLRRMELKP

SEQ ID NO: 224 - protein, 595 aa, Artificial sequence
  Synthetic polypeptide
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 225 - protein, 238 aa, Artificial sequence
  Synthetic polypeptide
LTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAH

SEQ ID NO: 226 - protein, 236 aa, Artificial sequence
  Synthetic polypeptide
LSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAH

SEQ ID NO: 227 - protein, 227 aa, Artificial Sequence
  Synthetic polypeptide
NPTVPLISHLVNIEPNPILTGYNPQCTPTEGYLMALVTDLANREIEGLVDWAARLPGYGMLPMDDQVNLIRTVWLDLLMLGLVWRSMEHRGEWLVFAPDLLMDRSLCRLSGMEYICTPMLEFARQFADLQVPQEVYVCLKALTLYTTAVSRLQDYRQVQRLQHEINEALAEACSSTFGFSPGNIARLMMIVSQVRQLSSLGVDHLNRLRGAETVSVEGLLREIVDEP

SEQ ID NO: 228 - protein, 238 aa, Artificial sequence
  Synthetic polypeptide
LSPEELISRIMEAEPPEIYLMKDMKKPFTEANVMMSLTNLADKELVHMISWAKKIPGFVELSLFDQVHLLECCWLEVLMLGLMWRSVNHPGKLIFSPDLCLSRDESSCVQGLVEIFDMLLAATSRFRELKLQREEYVCLKAMILLNSNMCLGSSEGGEDLQSRSKLLCLLDSVTDALVWAISKTGLSFQQRSTRLAHLLMLLSHIRHVSNKGMDHLHCMKMKKMVPLYDLLLEMLDAH

SEQ ID NO: 229 - protein, 238 aa, Artificial sequence
  Synthetic polypeptide
LTAEQMVSALLEAEPPIVYSEYDPNRPFNEASMMTLLTNLADRELVHMINWAKRVPGFVDLTLHDQVHLLECAWLEILMIGLVWRSMEHPGKLLFAPNLLLDRNQGKCVEGMVEIFDMLLATAARFRMMNLQGEEFVCLKSIILLNSGVYTFLSSTLKSLEERDYIHRVLDKITDTLIHLMAKSGLSLQQQHRRLAQLLLILSHIRHMSNKGMEHLYNMKCKNVVPLYDLLLEMLDAH

SEQ ID NO: 230 - protein, 238 aa, Artificial sequence
  Synthetic polypeptide
LTAEQLISALMEAEAPIVYSEHDSTKPLSEASMMTLLTNLADRELVHMINWAKRVPGFVDLTLHDQVHLLECAWLEILMVGLIWRSVEHPGKLSFAPNLLLDRNQGRCVEGLVEIFDMLVTTATRFRMMRLRGEEFICLKSIILLNSGVYTFLSSTLESLEDTDLIHIILDKIIDTLVHFMAKSGLSLQQQQRRLAQLLLILSHIRHMSNKGMEHLYSMKCKNVVPLYDLLLEMLDAH

SEQ ID NO: 231 - protein, 243 aa, Artificial sequence
  Synthetic polypeptide
QRLKALIDALDVKEGEHRGEENHPTGQQAGNWQEISNPELIESVSSLVDRELTGIICWGKKIPGYSKLSLNDQVLLMESTWLDLLILDLVWCSIRHKGEKLLLSGGVLVNRNTISNRRNNSSGDDMEVLEMCDQILSIATKFYEFDLQRREYLCLKAITLVHGSLKGLESDTQVRQLQDDLTDALMDVCSERHALGSRRPAKMLLLLSHLRQVSARASSHLGAVRNGLKVPLYDILLDILTDQ

SEQ ID NO: 232 - protein, 235 aa, Artificial sequence
  Synthetic polypeptide
LSSEQFLTCLLNAEPPNMTCHHDVSRPFTAERLMMLLTNLADRELVHMIGWAKKVPGFVQISLRDQVLLLESSWLEVLIMGLIWRSMSQPGKLVFASNLILDRDDGECVEGIFEIFDILLNIVQHFRELQVWMDEYVCLKAIILLNASMMVATSEKDGSRAKVQQLVEATTDTLVKCIARRSLATPEQFRRLSHLLTILSHIRHISNKGIQHMYSMKCKNLVPFYDLLLEMLDAH

SEQ ID NO: 233 - protein, 229 aa, Artificial sequence
  Synthetic polypeptide
SLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNV

SEQ ID NO: 234 - protein, 314 aa, Artificial sequence
  Synthetic polypeptide
SAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 235 - protein, 238 aa, Artificial sequence
  Synthetic polypeptide
LTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAH

SEQ ID NO: 236 - protein, 229 aa, Artificial sequence
  Synthetic polypeptide
SLALSLTADQMVSALLDAEPPILYSEYDPTRPFSEASMMGLLTNLAYRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNV

SEQ ID NO: 237 - protein, 314 aa, Artificial sequence
  Synthetic polypeptide
SAGDMRAANLWPSPLMIKRSKKNSLALSLTADQMVSALLDAEPPILYSEYDPTRPFSEASMMGLLTNLAYRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 238 - protein, 238 aa, Artificial sequence
  Synthetic polypeptide
LTADQMVSALLDAEPPILYSEYDPTRPFSEASMMGLLTNLAYRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAH

(19) United States
(12) Patent Application Publication
(10) Pub. No.: US 2021/0206826 A1
(43) Pub. Date: Jul. 8, 2021

(54) CONDITIONALLY REPRESSIBLE IMMUNE CELL RECEPTORS AND METHODS OF USE THEREOF

(71) Applicant: THE REGENTS OF THE UNIVERSITY OF CALIFORNIA, Oakland, CA (US)

(72) Inventors: Wendell A. LIM, San Francisco, CA (US); Kole T. ROYBAL, San Francisco, CA (US); Chia-Yung WU, San Francisco, CA (US); Jasper Z. WILLIAMS, San Francisco, CA (US)

(73) Assignee: The Regents of the University of California, Oakland, CA (US)

(21) Appl. No.: 15/777,592

(22) PCT Filed: Nov. 17, 2016
(86) PCT No.: PCT/US2016/062612

§ 371 Date: May 18, 2018

Related U.S. Application Data
(60) Provisional application No. 62/257,592, filed on Nov. 19, 2015.

(51) Int. Cl. C07K 14/725; C12N 15/86

(52) U.S. Cl. CPC C07K 14/7051; A61K 38/00; C07K 2319/03; C12N 15/86

(57) ABSTRACT

The present disclosure provides heteromeric, conditionally repressible synthetic immune cell receptors, nucleic acids expressing such receptors, cells expressing such nucleic acids and methods of making and using such receptors and nucleic acids. The present disclosure also provides methods of repressing immune cell activation attributable to a stimulatory synthetic immune cell receptor by dimerizing the stimulatory synthetic immune cell receptor with a synthetic immune cell repressor.

### CROSS-REFERENCE

This application claims the benefit of U.S. Provisional Patent Application No. 62/257,592, filed Nov. 19, 2015, which application is incorporated herein by reference in its entirety.

### STATEMENT REGARDING FEDERALLY SPONSORED RESEARCH

This invention was made with government support under Grant Nos. EY016546, P50 GM081879, R01 CA196277 and R01 GM055040 awarded by the National Institutes of Health. The government has certain rights in the invention.

### INCORPORATION BY REFERENCE OF SEQUENCE LISTING PROVIDED AS A TEXT FILE

A Sequence Listing is provided herewith as a text file, “UCSF-521WO_SeqList_ST25.txt” created on Nov. 17, 2016 and having a size of 108 KB. The contents of the text file are incorporated by reference herein in their entirety.

### INTRODUCTION

Artificial immune cell activation can be achieved through expression of various designer stimulating immune cell receptors including, e.g., synthetic chimeric antigen receptors (CAR) and engineered T cell Receptors (TCR). However, safe human testing and possibly even clinical use of such designer CAR and TCR immune cell stimulators requires fine control of the powerful stimulating activity of these highly engineered proteins and protein complexes. Such control is required in order to inhibit, halt or otherwise modulate immune cell activation when activation from the designer stimulating receptor is unwanted, becomes undesirable or is no longer necessary.

### SUMMARY

The present disclosure provides heteromeric, conditionally repressible synthetic immune cell receptors, nucleic acids expressing such receptors, cells expressing such nucleic acids and methods of making and using such receptors and nucleic acids. The present disclosure also provides methods of repressing immune cell activation attributable to a stimulatory synthetic immune cell receptor by dimerizing the stimulatory synthetic immune cell receptor with a synthetic immune cell repressor.

Provided is a heteromeric, conditionally repressible synthetic immune cell receptor (ICR) comprising: a synthetic stimulatory ICR comprising a first member of a dimerization pair linked to the synthetic stimulatory ICR; and a synthetic ICR repressor comprising a second member of the dimerization pair linked to an intracellular inhibitory domain.

Also provided is a conditionally repressible synthetic ICR, wherein the synthetic stimulatory ICR comprises an intracellular co-stimulatory domain. Also provided is such a conditionally repressible synthetic ICR, wherein the synthetic stimulatory ICR comprises an intracellular co-stimulatory domain and the intracellular co-stimulatory domain is selected from the group consisting of: 4-1BB (CD137), CD28, ICOS, OX-40, BTLA, CD27, CD30, GITR, and HVEM.

Also provided is a conditionally repressible synthetic ICR, wherein the first member of a dimerization pair is linked intracellularly to the synthetic stimulatory ICR and the second member of the dimerization pair is linked intracellularly to the intracellular inhibitory domain.

Also provided is a conditionally repressible synthetic ICR, wherein the synthetic ICR repressor further comprises a transmembrane domain. Also provided is a conditionally repressible synthetic ICR, wherein the second member of the dimerization pair is linked intracellularly to the transmembrane domain. Also provided is a conditionally repressible synthetic ICR, wherein the second member of the dimerization pair is extracellular and linked to the intracellular inhibitory domain by way of the transmembrane domain.

Also provided is a conditionally repressible synthetic ICR, wherein the stimulatory ICR binds a soluble antigen.

Also provided is a conditionally repressible synthetic ICR, wherein the stimulatory ICR binds a cell surface antigen, in some cases a peptide-major histocompatibility complex (peptide-MHC).

Also provided is a conditionally repressible synthetic ICR, wherein the intracellular inhibitory domain is an inhibitory domain derived from a protein selected from the group consisting of: PD-1, CTLA4, HPK1, SHP1, SHP2, Sts1 and Csk.

Also provided is a conditionally repressible synthetic ICR, wherein the synthetic stimulatory ICR comprises an intracellular signaling domain selected from the group consisting of: a CD3-zeta signaling domain, a ZAP70 signaling domain and an immunoreceptor tyrosine-based activation motif (ITAM).

Also provided is a conditionally repressible synthetic ICR, wherein the first and second members of the dimerization pair form a homodimer in the presence of a small molecule dimerizer.

Also provided is a conditionally repressible synthetic ICR, wherein the first and second members of the dimerization pair form a heterodimer in the presence of a small molecule dimerizer.

Also provided is a conditionally repressible synthetic ICR, wherein the dimerization pair is a dimerization pair responsive to a small molecule selected from the group consisting of: rapamycin or an analog thereof, gibberellic acid or an analog thereof, coumermycin or an analog thereof, methotrexate or an analog thereof, abscisic acid or an analog thereof and tamoxifen or an analog thereof.

Also provided is a conditionally repressible synthetic ICR, wherein the synthetic stimulatory ICR is a synthetic chimeric antigen receptor (CAR) or portion thereof.

Also provided is a conditionally repressible synthetic ICR of any one of the preceding claims, wherein the synthetic stimulatory ICR is a synthetic T cell receptor (TCR) or portion thereof.

Also provided is a mammalian cell genetically modified to produce a heteromeric, conditionally repressible synthetic ICR. Also provided is a T cell genetically modified to produce a heteromeric, conditionally repressible synthetic ICR.

Also provided is a nucleic acid comprising a nucleotide sequence encoding a heteromeric, conditionally repressible synthetic ICR. Also provided is a nucleic acid comprising a nucleotide sequence encoding a heteromeric, conditionally repressible synthetic ICR, wherein the nucleotide sequence is operably linked to a T cell specific promoter.

Also provided is a recombinant expression vector comprising a nucleic acid comprising a nucleotide sequence encoding a heteromeric, conditionally repressible synthetic ICR. Also provided is an in vitro transcribed RNA comprising a nucleotide sequence encoding a heteromeric, conditionally repressible synthetic ICR.

Also provided is a method of repressing T cell activation, the method comprising: contacting a T cell that expresses a heteromeric, conditionally repressible synthetic ICR and has been activated by binding of an antigen or epitope to the synthetic stimulatory ICR with a dimerizing agent; wherein, in the presence of the dimerizing agent, the first and second members of the dimerization pair dimerize and the intracellular inhibitory domain represses the activation of the T cell. Also provided is the method as described, wherein said contacting occurs in vivo.

Also provided is a method of making a cell, the method comprising genetically modifying a mammalian cell with an expression vector comprising nucleotide sequences encoding a conditionally repressible synthetic ICR, or genetically modifying a mammalian cell with an RNA comprising nucleotide sequences encoding a conditionally repressible synthetic ICR. Also provided is such a method, wherein said genetic modification is carried out ex vivo. Also provided is such a method, wherein the cell is a T lymphocyte, a stem cell, an NK cell, a progenitor cell, a cell derived from a stem cell, or a cell derived from a progenitor cell.

Also provided is a method of modulating treatment of a cancer in an individual, the method comprising: genetically modifying an immune cell or immune cell progenitor obtained from the individual with an expression vector comprising nucleotide sequences encoding a conditionally repressible synthetic ICR, wherein the synthetic stimulatory ICR is specific for an epitope on a cancer cell in the individual; treating the individual with the genetically modified immune cell, immune cell progenitor or progeny thereof under conditions sufficient for killing of the cancer cell; and modulating the treatment of the individual by administering to the individual an effective amount of a dimerizing agent, wherein the dimerizing agent induces dimerization of the first and second members of the dimerization pair, wherein said dimerization provides for repression of the genetically modified immune cell, immune cell progenitor or progeny thereof. Also provide is such a method, wherein the genetic modification is carried out ex vivo and the treating comprises introducing the genetically modified immune cell, immune cell progenitor or progeny thereof into the individual.

Also provided is a method of repressing the activity of a host cell, the method comprising contacting an activated host cell with a dimerizing agent, wherein the host cell is genetically modified to produce a conditionally repressible synthetic ICR, and wherein, in the presence of the dimerizing agent the first and second dimerizing members of the conditionally repressible synthetic ICR dimerize and represses at least one activity of the activated host cell. Also provided is such a method, wherein the activity is selected from the group consisting of: proliferation, cell survival, apoptosis, gene expression, immune activation, cytokine/chemokine secretion and combinations thereof.

Also provided is a heteromeric, conditionally repressible synthetic chimeric antigen receptor (CAR) comprising: a synthetic stimulatory CAR comprising: i) a extracellular recognition domain; ii) a transmembrane domain linked to the extracellular recognition domain; iii) a first member of a dimerization pair linked to the transmembrane domain; and iv) an intracellular stimulation domain; and a synthetic CAR repressor comprising: i) a second member of the dimerization pair; and ii) an intracellular inhibitory domain linked to the second member of the dimerization pair. Also provided is such a heteromeric, conditionally repressible synthetic CAR, wherein the synthetic CAR repressor further comprises a transmembrane domain linked to the second member of the dimerization pair, the intracellular inhibitory domain or both.

Also provided is a heteromeric, conditionally repressible synthetic T cell receptor (TCR) comprising: a synthetic stimulatory TCR comprising: i) a transmembrane domain; ii) a first member of a dimerization pair linked to the transmembrane domain; iii) an engineered TCR polypeptide comprising at least one TCR alpha or beta chain, wherein the at least one TCR alpha or beta chain is linked to the transmembrane domain or the first member of a dimerization pair; and a synthetic TCR repressor comprising: i) a second member of the dimerization pair; and ii) an intracellular inhibitory domain linked to the second member of the dimerization pair. Also provided is such a heteromeric, conditionally repressible synthetic TCR, wherein the synthetic TCR repressor further comprises a transmembrane domain linked to the second member of the dimerization pair, the intracellular inhibitory domain or both. Also provided is such a heteromeric, conditionally repressible synthetic TCR, wherein the engineered TCR polypeptide further comprises a TCR gamma chain.

### BRIEF DESCRIPTION OF THE DRAWINGS

FIG. 1 depicts a schematic of one embodiment of a heteromeric, conditionally repressible CAR as described herein.

FIG. 2 depicts a schematic representation of an assay for evaluating repression of immune cell activation by a heteromeric, conditionally repressible CAR as described herein.

FIG. 3 depicts repression of immune cell activation by a heteromeric, conditionally repressible CAR under varied levels of antigen stimulation as described herein.

FIG. 4 provides a graphical representation showing repression of immune cell activation by a heteromeric, conditionally repressible CAR under varied levels of antigen stimulation as described herein.

FIG. 5 provides an overlay graphical representation showing repression of immune cell activation by a heteromeric, conditionally repressible CAR under varied levels of antigen stimulation as described herein.

FIG. 6A-6B depicts repression of immune cell activation by a heteromeric, conditionally repressible CAR as assayed by ELISA.

FIG. 7 depicts repression of immune cell activation by a heteromeric, conditionally repressible CARs having various inhibitory domains and combinations thereof.

FIG. 8 depicts repression of immune cell activation by a heteromeric, conditionally repressible CARs having various inhibitory domains and combinations thereof.

FIG. 9 provides a graphical representation showing the repression of immune cell activation by a heteromeric, conditionally repressible CARs having various inhibitory domains and combinations thereof.

FIG. 10 depicts a schematic representation of an antigen expressing target cell assay for evaluating repression of immune cell activation by a heteromeric, conditionally repressible CAR as described herein.

FIG. 11A-11C depicts the results of repression of immune cell activation by a heteromeric, conditionally repressible CAR in an antigen expressing target cell assay.

FIG. 12 provides a graphical representation showing the repression of immune cell activation by a heteromeric, conditionally repressible CAR in an antigen expressing target cell assay.

FIG. 13 provides a schematic representation of various heteromeric, conditionally repressible ICR component configurations as described herein.

FIG. 14 provides a schematic representation of various engineered TCR variants for use in certain embodiments of heteromeric, conditionally repressible ICRs as described herein.

FIG. 15 provides Table 1.

FIG. 16 depicts a schematic representation of one embodiment of a repressible (i.e., “OFF-switch”) CAR in various states as used in a method of repressing T cell activation.

FIG. 17 depicts certain methods of repressing a repressible ICR by administering a dimerizer in two different exemplary and non-limiting clinical scenarios.

FIG. 18 depicts the rapalog concentration response of two different repressible ICRs in a CD69 expression assay.

FIG. 19 depicts the influence of rapalog timing on the CD69 expression response for two different repressible ICRs.

FIG. 20 depicts the rapalog concentration response of two different repressible ICRs in a T cell proliferation assay.

FIG. 21 provides results demonstrating titratable inhibition of IL-2 secretion using an OFF-switch CAR as described herein.

FIG. 22 provides results demonstrating titratable inhibition of INF-γ secretion using an OFF-switch CAR as described herein.

FIG. 23 depicts results demonstrating titratable inhibition of INF-α secretion using an OFF-switch CAR as described herein.

FIG. 24 depicts results demonstrating titratable inhibition of IL-10 secretion using an OFF-switch CAR as described herein.

FIG. 25 depicts results demonstrating titratable inhibition of IL-6 secretion using an OFF-switch CAR as described herein.

FIG. 26 provides a schematic representation of an assay for specific cell killing using OFF-switch CAR CD8 T cells as described herein.

FIG. 27 depicts the results of an assay for specific cell killing using PD1 OFF-switch CAR CD8 T cells as described herein.

FIG. 28 depicts the results of an assay for specific cell killing using CTLA4 OFF-switch CAR CD8 T cells as described herein

FIG. 29 provide calculated lysis specificity values relevant to the PD1 OFF-switch CAR CD8 T cell results provided in FIG. 27.

FIG. 30 provide calculated lysis specificity values relevant to the CTLA4 OFF-switch CAR CD8 T cell results provided in FIG. 28.

FIG. 31 provides a table showing various possible combinations of LBD, co-regulator, and dimerization agent.

FIG. 32 depicts the amino acid full-length estrogen receptor-alpha (ERα) amino acid sequence (SEQ ID NO:224).

FIG. 33 depicts multiple sequence alignment of LBD of ERα of various species. amino acid sequences of estrogen receptor-alpha (ERα) (SEQ ID NOs:225-232).

FIG. 34 depicts an amino acid sequence of LBD of ERα (SEQ ID NO:233).

FIG. 35 depicts an amino acid sequence of LBD of ERα (SEQ ID NO:234).

FIG. 36 depicts an amino acid sequence of LBD of ERα (SEQ ID NO:235).

FIG. 37 depicts an amino acid sequence of LBD of ERα (SEQ ID NO:236).

FIG. 38 depicts an amino acid sequence of LBD of ERα (SEQ ID NO:237).

FIG. 39 depicts an amino acid sequence of LBD of ERα (SEQ ID NO:238).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “domain” and “motif”, used interchangeably herein, refer to both structured domains having one or more particular functions and unstructured segments of a polypeptide that, although unstructured, retain one or more particular functions. For example, a structured domain may encompass but is not limited to a continuous or discontinuous plurality of amino acids, or portions thereof, in a folded polypeptide that comprise a three-dimensional structure which contributes to a particular function of the polypeptide. In other instances, a domain may include an unstructured segment of a polypeptide comprising a plurality of two or more amino acids, or portions thereof, that maintains a particular function of the polypeptide unfolded or disordered. Also encompassed within this definition are domains that may be disordered or unstructured but become structured or ordered upon association with a target or binding partner. Non-limiting examples of intrinsically unstructured domains and domains of intrinsically unstructured proteins are described, e.g., in Dyson & Wright. Nature Reviews Molecular Cell Biology 6:197-208.

The term “module”, as used herein, refers to a contiguous polypeptide sequence, or fragment thereof, that is associated with some function, particularly a biological function.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

The term “gene” refers to a particular unit of heredity present at a particular locus within the genetic component of an organism. A gene may be a nucleic acid sequence, e.g., a DNA or RNA sequence, present in a nucleic acid genome, a DNA or RNA genome, of an organism and, in some instances, may be present on a chromosome. A gene can be a DNA sequence that encodes for an mRNA that encodes a protein. A gene may be comprised of a single exon and no introns, or can include multiple exons and one or more introns. One of two or more identical or alternative forms of a gene present at a particular locus is referred to as an “allele” and, for example, a diploid organism will typically have two alleles of a particular gene. New alleles of a particular gene may be generated either naturally or artificially through natural or induced mutation and propagated through breeding or cloning. A gene or allele may be isolated from the genome of an organism and replicated and/or manipulated or a gene or allele may be modified in situ through gene therapy methods. The locus of a gene or allele may have associated regulatory elements and gene therapy, in some instances, may include modification of the regulatory elements of a gene or allele while leaving the coding sequences of the gene or allele unmodified.

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

As used herein, the term “hinge region” refers to a flexible polypeptide connector region (also referred to herein as “hinge” or “spacer”) providing structural flexibility and spacing to flanking polypeptide regions and can consist of natural or synthetic polypeptides. A “hinge region” derived from an immunoglobulin (e.g., IgG1) is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include complete hinge region derived from an antibody of a different class or subclass from that of the CH1 domain. The term “hinge region” can also include regions derived from CD8 and other receptors that provide a similar function in providing flexibility and spacing to flanking regions.

An “isolated” polypeptide or nucleic acid is one that has been identified and separated and/or recovered from a component of its natural environment. Contaminant components of its natural environment are materials that would interfere with diagnostic or therapeutic uses for the polypeptide or nucleic acid, and may include enzymes, hormones, and other proteinaceous or nonproteinaceous solutes. In some embodiments, a polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

As used herein, the term “immune cells” generally includes white blood cells (leukocytes) which are derived from hematopoietic stem cells (HSC) produced in the bone marrow. “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4+ cells), cytotoxic T-cells (CD8+ cells), T-regulatory cells (Treg) and gamma-delta T cells.

A “cytotoxic cell” includes CD8+ T cells, natural-killer (NK) cells, and neutrophils, which cells are capable of mediating cytotoxicity responses.

As used herein, the term “stem cell” generally includes pluripotent or multipotent stem cells. “Stem cells” includes, e.g., embryonic stem cells (ES); mesenchymal stem cells (MSC); induced-pluripotent stem cells (iPS); and committed progenitor cells (hematopoietic stem cells (HSC); bone marrow derived cells, neural progenitor cells, etc.).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

As used herein, the term “heteromeric” refers to a polypeptide or protein that contains more than one kind of subunit. Such heteromeric polypeptides may, in some instances, be referred to as “a heteromer”. Heteromeric polypeptides may contain two or more different polypeptides, wherein different polypeptides are defined at least as two polypeptides that are not identical, however, such different polypeptides may or may not include one or more portions of similar and/or identical amino acid sequence. In some instances, the two or more polypeptides of a heteromer share no identical amino acid sequence or share no identical domains. A heteromer may, in some instances, consist of two different polypeptides or two different types of polypeptides and may be referred to as a heterodimer. In some instances, a heteromer may consist of three different polypeptides or three different types of polypeptides and may be referred to as a heterotrimer. In some instances, a heteromer may consist of two or more different polypeptides or two or more different types of polypeptides, including but not limited to, e.g., three or more different polypeptides, four or more different polypeptides, five or more different polypeptides, six or more different polypeptides, seven or more different polypeptides, eight or more different polypeptides, etc.

In comparison, a “homomer” refers to a polypeptide or protein that contains only one kind of subunit. For example, in some instances, a homomer may consist of two of the same polypeptides and may be referred to as a homodimer. A homomer may consist of two or more of the same polypeptides, including but not limited to, e.g., three or more of the same polypeptides, four or more of the same polypeptides, five or more of the same polypeptides, six or more of the same polypeptides, seven or more of the same polypeptides, eight or more of the same polypeptides, etc.

The term “synthetic” as used herein generally refers to an artificially derived polypeptide or polypeptide encoding nucleic acid that is not naturally occurring. Such synthetic polypeptides and/or nucleic acids may be assembled de novo from basic subunits including, e.g., single amino acids, single nucleotides, etc., or may be derived from pre-existing polypeptides or polynucleotides, whether naturally or artificially derived, e.g., as through recombinant methods.

The term “recombinant”, as used herein describes a nucleic acid molecule, e.g., a polynucleotide of genomic, cDNA, viral, semisynthetic, and/or synthetic origin, which, by virtue of its origin or manipulation, is not associated with all or a portion of the polynucleotide sequences with which it is associated in nature. The term recombinant as used with respect to a protein or polypeptide means a polypeptide produced by expression from a recombinant polynucleotide. The term recombinant as used with respect to a host cell or a virus means a host cell or virus into which a recombinant polynucleotide has been introduced. Recombinant is also used herein to refer to, with reference to material (e.g., a cell, a nucleic acid, a protein, or a vector) that the material has been modified by the introduction of a heterologous material (e.g., a cell, a nucleic acid, a protein, or a vector).

“Operably linked” refers to a juxtaposition wherein the components so described are in a relationship permitting them to function in their intended manner. For instance, a promoter is operably linked to a coding sequence if the promoter affects its transcription or expression.

A “biological sample” encompasses a variety of sample types obtained from an individual or a population of individuals and can be used in a diagnostic, monitoring or screening assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by mixing or pooling of individual samples, treatment with reagents, solubilization, or enrichment for certain components, such as cells, polynucleotides, polypeptides, etc. The term “biological sample” encompasses a clinical sample, and also includes cells in culture, cell supernatants, cell lysates, serum, plasma, biological fluid, and tissue samples. The term “biological sample” includes urine, saliva, cerebrospinal fluid, interstitial fluid, ocular fluid, synovial fluid, blood fractions such as plasma and serum, and the like. The term “biological sample” also includes solid tissue samples, tissue culture samples, and cellular samples.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a dimerizer” includes a plurality of such dimerizers and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides heteromeric, conditionally repressible synthetic immune cell receptors, nucleic acids comprising nucleotide sequences encoding such receptors, cells expressing such nucleic acids and methods of making and using such receptors and nucleic acids. The present disclosure also provides methods of repressing immune cell activation attributable to a stimulatory synthetic immune cell receptor by dimerizing the stimulatory synthetic immune cell receptor with a synthetic immune cell repressor.

### Heteromeric, Conditionally Repressible Synthetic Immune Cell Receptor

The present disclosure provides heteromeric, conditionally repressible synthetic immune cell receptors (ICR). The heteromeric, conditionally repressible synthetic ICR will generally include a synthetic stimulatory ICR and a synthetic ICR repressor configured such that upon introduction of a dimerizing agent the synthetic ICR repressor dimerizes with the synthetic stimulatory ICR to repress activation due to the synthetic stimulatory ICR.

The configuration of the heteromeric, conditionally repressible synthetic ICR will vary depending on the particular context within which repression of a synthetic stimulatory ICR is desired. In some instances, the stimulatory portion of the heteromeric, conditionally repressible synthetic ICR may be referred to as Part 1 of the heteromeric, conditionally repressible synthetic ICR. In some instances, the repressor portion of the heteromeric, conditionally repressible synthetic ICR may be referred to as Part 2 of the heteromeric, conditionally repressible synthetic ICR. Thus, a heteromeric, conditionally repressible synthetic ICR collectively refers to a multi-modular protein or protein complex that includes various modules including the stimulatory portion (e.g., synthetic stimulatory ICR, Part 1, etc.) and the repressor portion (e.g., synthetic ICR repressor, Part 2, etc.) whether or not the various modules are or are not present or were or were not present at some point within the same protein and whether or not the various modules are expressed from the same or different nucleic acid constructs.

One of skill in the art will readily recognize from the instant disclosure that first and second parts (e.g., stimulatory and inhibitory parts) will individually include first and second portions of a dimerizing pair and that such portions of the dimerizing pair may be interchangeable between the first and second parts of the heteromeric, conditionally repressible synthetic ICR. One of skill in the art will also readily recognize from the instant disclosure that individual domains of heteromeric, conditionally repressible synthetic ICR may be rearranged in many instances, in order and/or orientation, while maintain the functions of being activatable and repressible as described herein. As such, description of a particular configuration of a heteromeric, conditionally repressible synthetic ICR described herein also includes wherein the modules of the heteromeric, conditionally repressible synthetic ICR are rearranged without abolishing the primary functions of the heteromeric, conditionally repressible synthetic ICR. Such rearrangements may also include the inclusion or exclusion of particular optional modules (including e.g., linkers, reporters, etc.) that do not result in abolishment of the primary functions of the heteromeric, conditionally repressible synthetic ICR due to their inclusion or exclusion from the heteromeric, conditionally repressible synthetic ICR.

### Synthetic Stimulatory ICR

As described herein, a heteromeric, conditionally repressible synthetic ICR includes a synthetic stimulatory ICR, also referred to herein as a “stimulatory ICR” or “stimulatory part” for simplicity. Such stimulatory ICRs will vary depending on the particular context of immune cell stimulation to which the construct is directed and will generally function to mediate activation of the immune cell expressing the stimulatory ICR. Thus, a stimulatory ICR includes an extracellular domain that upon reception of a specific signal functions to transduce the signal intracellularly to activate the immune cell expressing the stimulatory ICR.

In some instances, the extracellular component of a stimulatory ICR therefore may include an extracellular recognition domain, described in more detail below, that contains one member of a specific binding pair. Specific binding pairs include, but are not limited to, antigen-antibody binding pairs; ligand-receptor binding pairs; and the like. Thus, a member of a specific binding pair suitable for use in an extracellular recognition domain of the present disclosure includes an antigen; an antibody; a ligand; and a ligand-binding receptor.

In some instances, the extracellular component of a stimulatory ICR may include two or more extracellular recognition domains each specific for a particular binding partner (e.g., antigen). The number of extracellular recognition domains present in the extracellular component of a stimulatory ICR that recognizes multiple binding partners may likewise vary and may range from 2 to 10 or more including but not limited to e.g., 2, 3, 4, 5, 6, 7, 8, 9, 10, etc. Accordingly, in some instances, the number of different antigens that may be recognized by the extracellular component of a stimulatory ICR that recognizes multiple binding partners may vary and may range from 2 to 10 or more including but not limited to e.g., 2, 3, 4, 5, 6, 7, 8, 9, 10, etc. An extracellular component of a stimulatory ICR that recognizes multiple binding partners may, in some instances, be multi-specific including but not limited to e.g., bispecific, trispecific, and the like. Any combination of members of various specific binding pairs may find use in such multi-specific stimulatory ICRs, including e.g., combinations of those described herein. Such multi-specific stimulatory ICRs may essentially be an “OR-gate” where activation of the stimulatory ICR may be triggered by binding the first antigen or the second antigen or the third antigen, etc. In some instances, the multi-specific stimulatory ICRs may be a bispecific OR-gate. Examples of extracellular recognition domains the may find use in a multi-specific stimulatory ICR include but are not limited to e.g., the anti-CD19 and anti-CD20 extracellular binding domains of the CD19-CD20 bispecific CAR described in PCT International Pub. No. WO 2016100232 A1, the disclosure of which is incorporated herein by reference in its entirety.

A stimulatory ICR further includes one or more intracellular stimulation domains that, upon activation of one or more extracellular domains, mediates intracellular signaling leading to activation of the immune cell expressing the stimulatory ICR. Domains useful as signaling domains will vary depending on the particular context of immune cell activation, including e.g., the particular type of cell to be activated and the desired degree of activation. Exemplary non-limited examples of stimulatory domains, described in greater detail below, include but are not limited to domains and motifs thereof derived from immune stimulatory molecules including, e.g., co-stimulatory molecules, immune receptors and the like.

In some instances, stimulatory ICRs may be or may be derived from engineered or synthetic immune regulatory constructs designed for therapeutic immune system modulation including but not limited to e.g., chimeric antigen receptors (CARs) and derivatives, engineered T cell receptors (TCRs) and derivatives and the like. Engineered CARs, TCRs and derivatives thereof useful as the basis for a synthetic ICR include those CARs, TCRs and derivatives thereof that are activatable, e.g., are activated upon binding of a binding partner to the CAR, TCR or derivative thereof, and upon activation transduce the signal intracellularly to activate the immune cell expressing the CAR, TCR or derivative thereof. In some instances, a stimulatory ICR may be conditionally activatable such that activation upon binding of a binding partner to the stimulatory ICR requires an additional event for transduction of the activation signal including e.g., dimerization of components of the stimulatory ICR, e.g., as described in PCT/US2014/016527, the disclosure of which is incorporated herein by reference in its entirety.

A stimulatory ICR further includes, as described in more detail below, a domain of a dimerization pair. Useful dimerization domains will vary depending on the desired dimerizer and the desired relative position of the dimerization domain within the stimulatory ICR. Generally, the presence of a first domain of a dimerization pair within the stimulatory ICR mediates the dimerization, upon introduction of the dimerizer, with a second domain of the dimerization pair present in the ICR repressor such that upon dimerization the ICR repressor represses any immune cell activation due to the stimulatory ICR.

In some instances, a stimulatory ICR may further include additional domains. Such additional domains may be functional, e.g., they directly contribute to the immune cell activation function of the stimulatory ICR, or non-functional, e.g., they do not directly contribute to the activation function of the stimulatory ICR. Non-functional additional domains may include domains having various purposes that do not directly affect the ability of the stimulatory ICR to activate immune cell function including, but not limited to, e.g., structural functions, linker functions, etc.

### Chimeric Antigen Receptor (CAR)

In some instances, a heteromeric, conditionally repressible synthetic ICR may include, in part or in whole, a CAR or may essentially be a modified CAR such that by modification the CAR is conditionally repressible. In such instances, the CAR containing heteromeric, conditionally repressible synthetic ICR may be referred to as a heteromeric, conditionally repressible synthetic CAR or, for simplicity, a repressible CAR. Any CAR having immune cell activation function may find use in a heteromeric, conditionally repressible synthetic ICR as described herein including but not limited to, e.g., those CAR variants described herein.

In some instances, a CAR may be modified for use as a component of a heteromeric, conditionally repressible synthetic ICR through introduction or insertion of a dimerization domain (e.g., a member of a dimerizer pair) into the CAR and, in such instances, following modification, the CAR may be referred to as a dimerizer-domain containing CAR or a dimerizable CAR.

A dimerizer domain may be inserted into the CAR amino acid sequence, e.g., by introducing a coding sequence for the dimerizer domain into the coding sequence of the CAR, at any convenient location provided the insertion does not negatively impact the primary functional domains of the CAR (including e.g., the extracellular recognition domain, the immune activation domain(s), etc.) and/or the negatively impact the dimerization function of the dimerizer domain.

In some instances, the dimerizer may be inserted into an extracellular portion of the CAR. In some instances the dimerizer may be inserted into an intracellular portion of the CAR. In some instances, the dimerizer may be inserted such that following insertion the dimerizer is linked to an extracellular recognition domain of the CAR. In some instances, the dimerizer may be inserted such that following insertion the dimerizer is linked to a transmembrane domain of the CAR. In some instances, the dimerizer may be inserted such that following insertion the dimerizer is linked to the extracellular side of a transmembrane domain of the CAR. In some instances, the dimerizer may be inserted such that following insertion the dimerizer is linked to the intracellular side of a transmembrane domain of the CAR. In some instances, the dimerizer may be inserted such that following insertion the dimerizer is linked to an immune stimulatory domain of the CAR. In some instances, the dimerizer may be inserted such that following insertion the dimerizer is linked to a co-stimulation domain of the CAR. In some instances, the dimerizer may be inserted such that following insertion the dimerizer is at the N-terminal end of the CAR. In some instances, the dimerizer may be inserted such that following insertion the dimerizer is at the C-terminal end of the CAR.

In instances where a heteromeric, conditionally repressible synthetic ICR includes, in part or in whole, or the heteromeric, conditionally repressible synthetic ICR is essentially a modified CAR, the CAR may contain an extracellular recognition domain, a stimulatory domain and a transmembrane domain. Such a CAR may optionally include linker regions and/or hinge regions. CARs as part of a heteromeric, conditionally repressible synthetic ICR may be encompassed within a single polypeptide or may be “split” across two or more polypeptides.

### Extracellular Recognition Domain

A repressible CAR includes a member of a specific binding pair. Specific binding pairs include, but are not limited to, antigen-antibody binding pairs; ligand-receptor binding pairs; and the like. Thus, a member of a specific binding pair suitable for use in a repressible CAR of the present disclosure includes an antigen; an antibody; a ligand; and a ligand-binding receptor.

Antigen Binding Domain

An antigen-binding domain suitable for use in a repressible CAR of the present disclosure can be any antigen-binding polypeptide, a wide variety of which are known in the art. In some instances, the antigen-binding domain is a single chain Fv (scFv). Other antibody based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some instances, T-cell receptor (TCR) based recognition domains such as single chain TCR (scTv, single chain two-domain TCR containing VαVβ) are also suitable for use. Such TCR recognition domains when present as a repressible engineered TCR rather than a component of a repressible CAR are described in more detail below.

An antigen-binding domain suitable for use in a repressible CAR of the present disclosure can have a variety of antigen-binding specificities. In some cases, the antigen-binding domain is specific for an epitope present in an antigen that is expressed by (synthesized by) a cancer cell, i.e., a cancer cell associated antigen. Antigens bound by an antigen-binding domain may or may not be presented in the context of MHC, e.g., antigens may be present outside the context of MHC such as in the case of a cell surface antigen or may be presented in the context of MHC such as in the case of a peptide-MHC. The cancer cell associated antigen can be an antigen associated with, e.g., a breast cancer cell, a B cell lymphoma, a Hodgkin lymphoma cell, an ovarian cancer cell, a prostate cancer cell, a mesothelioma, a lung cancer cell (e.g., a small cell lung cancer cell), a non-Hodgkin B-cell lymphoma (B-NHL) cell, an ovarian cancer cell, a prostate cancer cell, a mesothelioma cell, a lung cancer cell (e.g., a small cell lung cancer cell), a melanoma cell, a chronic lymphocytic leukemia cell, an acute lymphocytic leukemia cell, a neuroblastoma cell, a glioma, a glioblastoma, a medulloblastoma, a colorectal cancer cell, etc. A cancer cell associated antigen may also be expressed by a non-cancerous cell.

Non-limiting examples of antigens to which an antigen-binding domain of a subject repressible CAR can bind include, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like.

Ligand

In some cases, a member of a specific binding pair suitable for use in a subject repressible CAR is a ligand for a receptor. Ligands include, but are not limited to, cytokines (e.g., IL-13, etc.); growth factors (e.g., heregulin; vascular endothelial growth factor (VEGF); and the like); an integrin-binding peptide (e.g., a peptide comprising the sequence Arg-Gly-Asp); and the like.

Where the member of a specific binding pair in a subject repressible CAR is a ligand, the repressible CAR can be activated in the presence of a second member of the specific binding pair and repressed in the presence of the dimerizer agent, where the second member of the specific binding pair is a receptor for the ligand. For example, where the ligand is VEGF, the second member of the specific binding pair can be a VEGF receptor, including a soluble VEGF receptor. As another example, where the ligand is heregulin, the second member of the specific binding pair can be Her2.

Receptors

As noted above, in some cases, the member of a specific binding pair that is included in a subject repressible CAR is a receptor, e.g., a receptor for a ligand, a co-receptor, etc. The receptor can be a ligand-binding fragment of a receptor. Suitable receptors include, but are not limited to, a growth factor receptor (e.g., a VEGF receptor); a killer cell lectin-like receptor subfamily K, member 1 (NKG2D) polypeptide (receptor for MICA, MICB, and ULB6); a cytokine receptor (e.g., an IL-13 receptor; an IL-2 receptor; etc.); Her2; CD27; a natural cytotoxicity receptor (NCR) (e.g., NKP30 (NCR3/CD337) polypeptide (receptor for HLA-B-associated transcript 3 (BAT3) and B7-H6); etc.); etc.

Stimulatory Domain

A stimulatory domain suitable for use in a stimulatory CAR of a subject repressible ICR may be any functional unit of a polypeptide as short as a 3 amino acid linear motif and as long as an entire protein, where size of the stimulatory domain is restricted only in that the domain must be sufficiently large as to retain its function and sufficiently small so as to be compatible with the other components of the repressible CAR. Accordingly, a stimulatory domain may range in size from 3 amino acids in length to 1000 amino acids or more and, in some instances, can have a length of from about 30 amino acids to about 70 amino acids (aa), e.g., a stimulatory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, stimulatory domain can have a length of from about 70 aa to about 100 aa, from about 100 aa to about 200 aa, or greater than 200 aa.

In some instances, “co-stimulatory domains” find use as stimulatory domains of a repressible CAR of the present disclosure. Co-stimulation generally refers to a secondary non-specific activation mechanism through which a primary specific stimulation is propagated. Examples of co-stimulation include antigen nonspecific T cell co-stimulation following antigen specific signaling through the T cell receptor and antigen nonspecific B cell co-stimulation following signaling through the B cell receptor. Co-stimulation, e.g., T cell co-stimulation, and the factors involved have been described in Chen & Flies. Nat Rev Immunol (2013) 13(4):227-42, the disclosure of which is incorporated herein by reference in its entirety. Co-stimulatory domains are generally polypeptides derived from receptors. In some embodiments, co-stimulatory domains homodimerize. A subject co-stimulatory domain can be an intracellular portion of a transmembrane protein (i.e., the co-stimulatory domain can be derived from a transmembrane protein). Non-limiting examples of suitable co-stimulatory polypeptides include, but are not limited to, 4-1BB (CD137), CD28, ICOS, OX-40, BTLA, CD27, CD30, GITR, and HVEM. In some instances, a co-stimulatory domain, e.g., as used in repressible CAR of the instant disclosure may include a co-stimulatory domain listed in Table 1. In some instances, a co-stimulatory domain of a repressible CAR comprises a an amino acid sequence having at least about 75%, at least about 80%, at least about 85%, at least about 90%, at least about 95%, at least about 98%, or 100% amino acid sequence identity to a co-stimulatory domain as described herein.

In some instances, a stimulatory CAR may contain an intracellular signaling domain, e.g., a co-stimulatory domain, derived from an intracellular portion of a transmembrane protein listed in Table 1. For example, a suitable co-stimulatory domain can comprise an amino acid sequence having at least about 75%, at least about 80%, at least about 85%, at least about 90%, at least about 95%, at least about 98%, or 100% amino acid sequence identity to an amino acid sequence listed in Table 1. In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165, aa from about 165 aa to about 170 aa, from about 170 aa to about 175 aa, from about 175 aa to about 180 aa, from about 180 aa to about 185 aa, or from about 185 aa to about 190 aa.

In some cases, a repressible CAR may contain two more stimulatory domains, present on the same or different polypeptides. In some instances, where the repressible CAR contains two more stimulatory domains, the stimulatory domains may have substantially the same amino acid sequences. For example, in some cases, the first stimulatory domain comprises an amino acid sequence that is at least about 90%, at least about 95%, at least about 98%, at least about 99%, or 100%, identical to the amino acid sequence of the second stimulatory domain. In some instances, where the repressible CAR contains two more stimulatory domains, the stimulatory domains of the subject repressible CAR can have substantially the same length; e.g., the first and second stimulatory domains can differ in length from one another by fewer than 10 amino acids, or fewer than 5 amino acids. In some instances, where the repressible CAR contains two more stimulatory domains, the first and second stimulatory domains have the same length. In some instances, where the repressible CAR contains two more stimulatory domains, the two stimulatory domains are the same.

In some instances, a repressible CAR may contain an intracellular signaling domain, e.g., a co-stimulatory domain, derived from an intracellular portion of the transmembrane protein 4-1BB (also known as TNFRSF9; CD137; 4-1BB; CDw137; ILA; etc.). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence: KRGRKKLLYIFKQPFMRPVQTTQEEDGCSCRFPEEEEGGCEL (SEQ ID NO:8). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a repressible CAR may contain an intracellular signaling domain, e.g., a co-stimulatory domain, derived from an intracellular portion of the transmembrane protein CD28 (also known as Tp44). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence: FWVRSKRSRLLHSDYMNMTPRRPGPTRKHYQPYAPPRDFAAYRS (SEQ ID NO:66). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a repressible CAR may contain an intracellular signaling domain, e.g., a co-stimulatory domain, derived from an intracellular portion of the transmembrane protein ICOS (also known as AILIM, CD278, and CVID1). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence: TKKKYSSSVHDPNGEYMFMRAVNTAKKSRLTDVTL (SEQ ID NO:4). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a repressible CAR may contain an intracellular signaling domain, e.g., a co-stimulatory domain, derived from an intracellular portion of the transmembrane protein OX-40 (also known as TNFRSF4, RP5-902P8.3, ACT35, CD134, OX40, TXGP1L). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence: RRDQRLPPDAHKPPGGGSFRTPIQEEQADAHSTLAKI (SEQ ID NO:67). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a repressible CAR may contain an intracellular signaling domain, e.g., a co-stimulatory domain, derived from an intracellular portion of the transmembrane protein BTLA (also known as BTLA1 and CD272). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: 21)
CCLRRHQGKQNELSDTAGREINLVDAHLKSEQTEASTRQNSQVLLSETGI
YDNDPDLCFRMQEGSEVYSNPCLEENKPGIVYASLNHSVIGPNSRLARNV
KEAPTEYASICVRS.

In some instances, a repressible CAR may contain an intracellular signaling domain, e.g., a co-stimulatory domain, derived from an intracellular portion of the transmembrane protein CD27 (also known as S152, T14, TNFRSF7, and Tp55). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence:

(SEQ ID NO: 68)
HQRRKYRSNKGESPVEPAEPCRYSCPREEEGSTIPIQEDYRKPEPACSP.

In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a repressible CAR may contain an intracellular signaling domain, e.g., a co-stimulatory domain, derived from an intracellular portion of the transmembrane protein CD30 (also known as TNFRSF8, D1S166E, and Ki-1). For example, a suitable co-stimulatory domain can comprise an amino acid sequence having at least about 75%, at least about 80%, at least about 85%, at least about 90%, at least about 95%, at least about 98%, or 100% amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, from about 150 aa to about 160 aa, or from about 160 aa to about 185 aa of the following amino acid sequence:

(SEQ ID NO: 24)
RRACRKRIRQKLHLCYPVQTSQPKLELVDSRPRRSSTQLRSGASVTEPVA
EERGLMSQPLMETCHSVGAAYLESLPLQDASPAGGPSSPRDLPEPRVSTE
HTNNKIEKIYIMKADTVIVGTVKAELPEGRGLAGPAEPELEEELEADHTP
HYPEQETEPPLGSCSDVMLSVEEEGKEDPLPTAASGK.

In some instances, a repressible CAR may contain an intracellular signaling domain, e.g., a co-stimulatory domain, derived from an intracellular portion of the transmembrane protein GITR (also known as TNFRSF18, RP5-902P8.2, AITR, CD357, and GITR-D). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence: HIWQLRSQCMWPRETQLLLEVPPSTEDARSCQFPEEERGERSAEEKGRLGDLWV (SEQ ID NO:12). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a repressible CAR may contain an intracellular signaling domain, e.g., a co-stimulatory domain, derived from an intracellular portion of the transmembrane protein HVEM (also known as TNFRSF14, RP3-395M20.6, ATAR, CD270, HVEA, HVEM, LIGHTR, and TR2). For example, a suitable co-stimulatory domain can comprise an amino acid sequence having at least about 75%, at least about 80%, at least about 85%, at least about 90%, at least about 95%, at least about 98%, or 100% amino acid sequence identity to the following amino acid sequence: CVKRRKPRGDVVKVIVSVQRKRQEAEGEATVIEALQAPPDVTTVAVEETIPSFTGRSPN H (SEQ ID NO:69). In some of these embodiments, the co-stimulatory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a repressible CAR may contain an intracellular signaling domain that includes at least one (e.g., one, two, three, four, five, six, etc.) intracellular signaling motif. In some instances, the intracellular signaling motif may be an immunoreceptor tyrosine-based activation motif (ITAM). In some instances, the intracellular signaling motif, e.g., an ITAM motif is within an intracellular signaling domain derived from a signaling molecule that contains one or more ITAM motifs. In other instances, the ITAM is derived, e.g., synthetically produced, within an amino acid sequence de novo, e.g., through mutation of the amino acid sequence.

An ITAM motif is YX₁X₂L/I, where X₁ and X₂ are independently any amino acid (SEQ ID NO:70). In some cases, the intracellular signaling domain of a subject repressible CAR comprises 1, 2, 3, 4, or 5 ITAM motifs. In some cases, an ITAM motif is repeated twice in an intracellular signaling domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid (SEQ ID NO:71). In some cases, the intracellular signaling domain of a subject repressible CAR comprises 3 ITAM motifs.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12; FCER1G (Fc epsilon receptor I gamma chain); CD3D (CD3 delta); CD3E (CD3 epsilon); CD3G (CD3 gamma); CD3Z (CD3 zeta); and CD79A (antigen receptor complex-associated protein alpha chain).

In some cases, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). For example, a suitable intracellular signaling domain can comprise an amino acid sequence having at least about 75%, at least about 80%, at least about 85%, at least about 90%, at least about 95%, at least about 98%, or 100%, amino acid sequence identity to any of the following amino acid sequences (4 isoforms): MGGLEPCSRLLLLPLLLAVSGLRPVQAQAQSDCSCSTVSPGVLAGIVMGDLVLTVLIAL AVYFLGRLVPRGRGAAEAATRKQRITETESPYQELQGQRSDVYSDLNTQRPYYK (SEQ ID NO:72); MGGLEPCSRLLLLPLLLAVSGLRPVQAQAQSDCSCSTVSPGVLAGIVMGDLVLTVLIAL AVYFLGRLVPRGRGAAEATRKQRITETESPYQELQGQRSDVYSDLNTQRPYYK (SEQ ID NO:73); MGGLEPCSRLLLLPLLLAVSDCSCSTVSPGVLAGIVMGDLVLTVLIALAVYFLGRLVPR GRGAAEAATRKQRITETESPYQELQGQRSDVYSDLNTQRPYYK (SEQ ID NO:74); or MGGLEPCSRLLLLPLLLAVSDCSCSTVSPGVLAGIVMGDLVLTVLIALAVYFLGRLVPR GRGAAEATRKQRITETESPYQELQGQRSDVYSDLNTQRPYYK (SEQ ID NO:75), where the ITAM motifs are in bold and are underlined.

Likewise, a suitable intracellular signaling domain can comprise an ITAM motif-containing portion of the full length DAP12 amino acid sequence. Thus, a suitable intracellular signaling domain can comprise an amino acid sequence having at least about 75%, at least about 80%, at least about 85%, at least about 90%, at least about 95%, at least about 98%, or 100%, amino acid sequence identity to the following amino acid sequence: ESPYQELQGQRSDVYSDLNTQ (SEQ ID NO:76), where the ITAM motifs are in bold and are underlined.

In some cases, the intracellular signaling domain is derived from FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). For example, a suitable intracellular signaling domain can comprise an amino acid sequence having at least about 75%, at least about 80%, at least about 85%, at least about 90%, at least about 95%, at least about 98%, or 100% amino acid sequence identity to the following amino acid sequence: MIPAVVLLLLLLVEQAAALGEPQLCYILDAILFLYGIVLTLLYCRLKIQVRKAAITSYEKS DGVYTGLSTRNQETYETLKHEKPPQ (SEQ ID NO:77), where the ITAM motifs are in bold and are underlined.

Likewise, a suitable intracellular signaling domain can comprise an ITAM motif-containing portion of the full length FCER1G amino acid sequence. Thus, a suitable intracellular signaling domain can comprise an amino acid sequence having at least about 75%, at least about 80%, at least about 85%, at least about 90%, at least about 95%, at least about 98%, or 100%, amino acid sequence identity to the following amino acid sequence: DGVYTGLSTRNQETYETLKHE (SEQ ID NO:78), where the ITAM motifs are in bold and are underlined.

In some cases, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). For example, a suitable intracellular signaling domain can comprise an amino acid sequence having at least about 75%, at least about 80%, at least about 85%, at least about 90%, at least about 95%, at least about 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 170 aa, of either of the following amino acid sequences (2 isoforms): MEHSTFLSGLVLATLLSQVSPFKIPIEELEDRVFVNCNTSITWVEGTVGTLLSDITRLDLG KRILDPRGIYRCNGTDIYKDKESTVQVHYRMCQSCVELDPATVAGIIVTDVIATLLLALG VFCFAGHETGRLSGAADTQALLRNDQVYQPLRDRDDAQYSHLGGNWARNK (SEQ ID NO:79) or MEHSTFLSGLVLATLLSQVSPFKIPIEELEDRVFVNCNTSITWVEGTVGTLLSDITRLDLG KRILDPRGIYRCNGTDIYKDKESTVQVHYRTADTQALLRNDQVYQPLRDRDDAQYSH LGGNWARNK (SEQ ID NO:80), where the ITAM motifs are in bold and are underlined.

Likewise, a suitable intracellular signaling domain can comprise an ITAM motif-containing portion of the full length CD3 delta amino acid sequence. Thus, a suitable intracellular signaling domain can comprise an amino acid sequence having at least about 75%, at least about 80%, at least about 85%, at least about 90%, at least about 95%, at least about 98%, or 100%, amino acid sequence identity to the following amino acid sequence: DQVYQPLRDRDDAQYSHLGGN (SEQ ID NO:81), where the ITAM motifs are in bold and are underlined.

In some cases, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). For example, a suitable intracellular signaling domain can comprise an amino acid sequence having at least about 75%, at least about 80%, at least about 85%, at least about 90%, at least about 95%, at least about 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 205 aa, of the following amino acid sequence:

MQSGTHWRVLGLCLLSVGVWGQDGNEEMGGITQTPYKVSISGTTVILTCPQYP GSEILWQHNDKNIGGDEDDKNIGSDEDHLSLKEFSELEQSGYYVCYPRGSKPEDANFYL YLRARVCENCMEMDVMSVATIVIVDICITGGLLLLVYYWSKNRKAKAKPVTRGAGAG GRQRGQNKERPPPVPNPDYEPIRKGQRDLYSGLNQRRI (SEQ ID NO:82), where the ITAM motifs are in bold and are underlined.

Likewise, a suitable intracellular signaling domain can comprise an ITAM motif-containing portion of the full length CD3 epsilon amino acid sequence. Thus, a suitable intracellular signaling domain can comprise an amino acid sequence having at least about 75%, at least about 80%, at least about 85%, at least about 90%, at least about 95%, at least about 98%, or 100%, amino acid sequence identity to the following amino acid sequence: NPDYEPIRKGQRDLYSGLNQR (SEQ ID NO:83), where the ITAM motifs are in bold and are underlined.

In some cases, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). For example, a suitable intracellular signaling domain can comprise an amino acid sequence having at least about 75%, at least about 80%, at least about 85%, at least about 90%, at least about 95%, at least about 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 180 aa, of the following amino acid sequence: MEQGKGLAVLILAIILLQGTLAQSIKGNHLVKVYDYQEDGSVLLTCDAEAKNITWFKD GKMIGFLTEDKKKWNLGSNAKDPRGMYQCKGSQNKSKPLQVYYRMCQNCIELNAATI SGFLFAEIVSIFVLAVGVYFIAGQDGVRQSRASDKQTLLPNDQLYQPLKDREDDQYSHL QGNQLRRN (SEQ ID NO:84), where the ITAM motifs are in bold and are underlined.

Likewise, a suitable intracellular signaling domain can comprise an ITAM motif-containing portion of the full length CD3 gamma amino acid sequence. Thus, a suitable intracellular signaling domain can comprise an amino acid sequence having at least about 75%, at least about 80%, at least about 85%, at least about 90%, at least about 95%, at least about 98%, or 100%, amino acid sequence identity to the following amino acid sequence: DQLYQPLKDREDDQYSHLQGN (SEQ ID NO:85), where the ITAM motifs are in bold and are underlined.

In some cases, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). For example, a suitable intracellular signaling domain can comprise an amino acid sequence having at least about 75%, at least about 80%, at least about 85%, at least about 90%, at least about 95%, at least about 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (2 isoforms): MKWKALFTAAILQAQLPITEAQSFGLLDPKLCYLLDGILFIYGVILTALFLRVKFSRSAD APAYQQGQNQLYNELNLGRREEYDVLDKRRGRDPEMGGKPRRKNPQEGLYNELQKD KMAEAYSEIGMKGERRRGKGHDGLYQGLSTATKDTYDALHMQALPPR (SEQ ID NO:86) or MKWKALFTAAILQAQLPITEAQSFGLLDPKLCYLLDGILFIYGVILTALFLRVKFSRSAD APAYQQGQNQLYNELNLGRREEYDVLDKRRGRDPEMGGKPQRRKNPQEGLYNELQK DKMAEAYSEIGMKGERRRGKGHDGLYQGLSTATKDTYDALHMQALPPR(SEQ ID NO:87), where the ITAM motifs are in bold and are underlined.

Likewise, a suitable intracellular signaling domain can comprise an ITAM motif-containing portion of the full length CD3 zeta amino acid sequence. Thus, a suitable intracellular signaling domain can comprise an amino acid sequence having at least about 75%, at least about 80%, at least about 85%, at least about 90%, at least about 95%, at least about 98%, or 100%, amino acid sequence identity to any of the following amino acid sequences: RVKFSRSADAPAYQQGQNQLYNELNLGRREEYDVLDKRRGRDPEMGGKPRRKNPQE GLYNELQKDKMAEAYSEIGMKGERRRGKGHDGLYQGLSTATKDTYDALHMQALPPR (SEQ ID NO:88); NQLYNELNLGRREEYDVLDKR (SEQ ID NO:89); EGLYNELQKDKMAEAYSEIGMK (SEQ ID NO:90); or DGLYQGLSTATKDTYDALHMQ (SEQ ID NO:91), where the ITAM motifs are in bold and are underlined.

In some cases, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). For example, a suitable intracellular signaling domain can comprise an amino acid sequence having at least about 75%, at least about 80%, at least about 85%, at least about 90%, at least about 95%, at least about 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 150 aa, from about 150 aa to about 200 aa, or from about 200 aa to about 220 aa, of either of the following amino acid sequences (2 isoforms): MPGGPGVLQALPATIFLLFLLSAVYLGPGCQALWMHKVPASLMVSLGEDAHFQCPHNS SNNANVTWWRVLHGNYTWPPEFLGPGEDPNGTLIIQNVNKSHGGIYVCRVQEGNESY QQSCGTYLRVRQPPPRPFLDMGEGTKNRIITAEGIILLFCAVVPGTLLLFRKRWQNEKLG LDAGDEYEDENLYEGLNLDDCSMYEDISRGLQGTYQDVGSLNIGDVQLEKP (SEQ ID NO:92); or MPGGPGVLQALPATIFLLFLLSAVYLGPGCQALWMHKVPASLMVSLGEDAHFQCPHNS SNNANVTWWRVLHGNYTWPPEFLGPGEDPNEPPPRPFLDMGEGTKNRIITAEGIILLFC AVVPGTLLLFRKRWQNEKLGLDAGDEYEDENLYEGLNLDDCSMYEDISRGLQGTYQD VGSLNIGDVQLEKP (SEQ ID NO:93), where the ITAM motifs are in bold and are underlined.

Likewise, a suitable intracellular signaling domain can comprise an ITAM motif-containing portion of the full length CD79A amino acid sequence. Thus, a suitable intracellular signaling domain can comprise an amino acid sequence having at least about 75%, at least about 80%, at least about 85%, at least about 90%, at least about 95%, at least about 98%, or 100%, amino acid sequence identity to the following amino acid sequence: ENLYEGLNLDDCSMYEDISRG (SEQ ID NO:94), where the ITAM motifs are in bold and are underlined.

In some instances, a repressible CAR may contain an intracellular signaling domain derived from a DAP10/CD28 type signaling chain. Intracellular signaling domains suitable for use in a repressible CAR of the present disclosure include a DAP10/CD28 type signaling chain.

An example of a DAP10 signaling chain is the amino acid sequence is: RPRRSPAQDGKVYINMPGRG (SEQ ID NO:95). In some embodiments, a suitable intracellular signaling domain comprises an amino acid sequence having at least about 85%, at least about 90%, at least about 95%, at least about 98%, or at least about 99%, amino acid sequence identity to the entire length of the amino acid sequence

(SEQ ID NO: 95)
RPRRSPAQDGKVYINMPGRG.

An example of a CD28 signaling chain is the amino acid sequence is FWVLVVVGGVLACYSLLVTVAFIIFWVRSKRSRLLHSDYMNMTPRRPGPTRKHYQPY APPRDFAAYRS (SEQ ID NO:96). In some embodiments, a suitable intracellular signaling domain comprises an amino acid sequence having at least about 85%, at least about 90%, at least about 95%, at least about 98%, or at least about 99%, amino acid sequence identity to the entire length of the amino acid sequence

(SEQ ID NO: 96)
FWVLVVVGGVLACYSLLVTVAFIIFWVRSKRSRLLHSDYMNMTPRRPGPT
RKHYQPYAPPRDFAAYRS.

Intracellular signaling domains suitable for use in a CAR of the present disclosure include a ZAP70 polypeptide, e.g., a polypeptide comprising an amino acid sequence having at least about 85%, at least about 90%, at least about 95%, at least about 98%, at least about 99%, or 100%, amino acid sequence identity to a contiguous stretch of from about 300 amino acids to about 400 amino acids, from about 400 amino acids to about 500 amino acids, or from about 500 amino acids to 619 amino acids, of the following amino acid sequence:

(SEQ ID NO: 97)
MPDPAAHLPFFYGSISRAEAEEHLKLAGMADGLFLLRQCLRSLGGYVLSL
VHDVRFHHFPIERQLNGTYAIAGGKAHCGPAELCEFYSRDPDGLPCNLRK
PCNRPSGLEPQPGVFDCLRDAMVRDYVRQTWKLEGEALEQAIISQAPQVE
KLIATTAHERMPWYHSSLTREEAERKLYSGAQTDGKFLLRPRKEQGTYAL
SLIYGKTVYHYLISQDKAGKYCIPEGTKFDTLWQLVEYLKLKADGLIYCL
KEACPNSSASNASGAAAPTLPAHPSTLTHPQRRIDTLNSDGYTPEPARIT
SPDKPRPMPMDTSVYESPYSDPEELKDKKLFLKRDNLLIADIELGCGNFG
SVRQGVYRMRKKQIDVAIKVLKQGTEKADTEEMMREAQIMHQLDNPYIVR
LIGVCQAEALMLVMEMAGGGPLHKFLVGKREEIPVSNVAELLHQVSMGMK
YLEEKNFVHRDLAARNVLLVNRHYAKISDFGLSKALGADDSYYTARSAGK
WPLKWYAPECINFRKFSSRSDVWSYGVTMWEALSYGQKPYKKMKGPEVMA
FIEQGKRMECPPECPPELYALMSDCWIYKWEDRPDFLTVEQRMRACYYSL
ASKVEGPPGSTQKAEAACA.

### Transmembrane Domain

Any transmembrane (TM) domain that provides for insertion of a polypeptide into the cell membrane of a eukaryotic (e.g., mammalian) cell is suitable for use. As one non-limiting example, the TM sequence IYIWAPLAGTCGVLLLSLVITLYC (SEQ ID NO:98) can be used. Additional non-limiting examples of suitable TM sequences include:

a) CD8 beta derived:
(SEQ ID NO: 99)
LGLLVAGVLVLLVSLGVAIHLCC;
b) CD4 derived:
(SEQ ID NO: 100)
ALIVLGGVAGLLLFIGLGIFFCVRC;
c) CD3 zeta derived:
(SEQ ID NO: 101)
LCYLLDGILFIYGVILTALFLRV;
d) CD28 derived:
(SEQ ID NO: 102)
WVLVVVGGVLACYSLLVTVAFIIFWV;
e) CD134 (OX40) derived:
(SEQ ID NO: 103)
VAAILGLGLVLGLLGPLAILLALYLL;
and
f) CD7 derived:
(SEQ ID NO: 104)
ALPAALAVISFLLGLGLGVACVLA.

### Linkers

In some cases, a subject repressible CAR includes a linker between any two adjacent domains. For example, a linker can be disposed between the transmembrane domain and the first intracellular functional domain, e.g., a co-stimulatory domain, of the repressible CAR. As another example, a linker can be disposed between a first intracellular functional domain and the member of the dimerization domain of the repressible CAR. As another example, a linker can be disposed between the member of the dimerization domain and a second intracellular functional domain, e.g., an immune cell activation domain. As another example, a linker can be disposed between any domain of the repressible CAR and any additional domain including e.g., a domain not involved in the primary immune activation functions of the CAR including but not limited to e.g., a reporter domain, a tag domain, etc.

Linkers may be utilized in a suitable configuration in the repressible CAR provided they do not abolish the primary activities of the repressible CAR including, e.g., the ability of the repressible CAR to become activated upon extracellular binding, the ability of the dimerization domain of the repressible CAR to bind the dimerization domain of the ICR repressor.

A linker peptide may have any of a variety of amino acid sequences. Proteins can be joined by a spacer peptide, generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins. Peptide linkers with a degree of flexibility can be used. The linking peptides may have virtually any amino acid sequence, bearing in mind that suitable linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, (GSGGS)ₙ (SEQ ID NO:105) and (GGGS)ₙ (SEQ ID NO:106), where n is an integer of at least one), glycine-alanine polymers, alanine-serine polymers, and other flexible linkers known in the art. Glycine and glycine-serine polymers are of interest since both of these amino acids are relatively unstructured, and therefore may serve as a neutral tether between components. Glycine polymers are of particular interest since glycine accesses significantly more phi-psi space than even alanine, and is much less restricted than residues with longer side chains (see Scheraga, Rev. Computational Chem. 11173-142 (1992)). Exemplary flexible linkers include, but are not limited GGSG (SEQ ID NO:107), GGSGG (SEQ ID NO:108), GSGSG (SEQ ID NO:109), GSGGG (SEQ ID NO:110), GGGSG (SEQ ID NO:111), GSSSG (SEQ ID NO:112), and the like. The ordinarily skilled artisan will recognize that design of a peptide conjugated to any elements described above can include linkers that are all or partially flexible, such that the linker can include a flexible linker as well as one or more portions that confer less flexible structure.

### Hinge Regions

In some cases, the first polypeptide of a subject repressible CAR comprises a hinge region (also referred to herein as a “spacer”), where the hinge region is interposed between the antigen-binding domain and the transmembrane domain. In some cases, the hinge region is an immunoglobulin heavy chain hinge region. In some cases, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

Suitable spacers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary spacers include glycine polymers (G)ₙ, glycine-serine polymers (including, for example, (GS)ₙ, (GSGGS)ₙ (SEQ ID NO:113) and (GGGS)ₙ (SEQ ID NO:114), where n is an integer of at least one), glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary spacers can comprise amino acid sequences including, but not limited to, GGSG (SEQ ID NO:107), GGSGG (SEQ ID NO:108), GSGSG (SEQ ID NO:109), GSGGG (SEQ ID NO:110), GGGSG (SEQ ID NO:111), GSSSG (SEQ ID NO:112), and the like.

In some cases, e.g., when the stimulatory ICR portion of a repressible CAR is split between two or more polypeptides the repressible CAR may include a hinge region that includes at least one cysteine. For example, in some cases, the hinge region can include the sequence Cys-Pro-Pro-Cys (SEQ ID NO:115). If present, a cysteine in the hinge region of a first polypeptide, e.g., a first portion of a repressible CAR, can be available to form a disulfide bond with a hinge region in a second polypeptide, e.g., a second portion of a repressible CAR.

Immunoglobulin hinge region amino acid sequences are known in the art; see, e.g., Tan et al. (1990) Proc. Natl. Acad. Sci. USA 87:162; and Huck et al. (1986) Nucl. Acids Res. 14:1779. As non-limiting examples, an immunoglobulin hinge region can include one of the following amino acid sequences: DKTHT (SEQ ID NO:116); CPPC (SEQ ID NO:115); CPEPKSCDTPPPCPR (SEQ ID NO:117) (see, e.g., Glaser et al. (2005) J. Biol. Chem. 280:41494); ELKTPLGDTTHT (SEQ ID NO:118); KSCDKTHTCP (SEQ ID NO:119); KCCVDCP (SEQ ID NO:120); KYGPPCP (SEQ ID NO:121); EPKSCDKTHTCPPCP (SEQ ID NO:122) (human IgG1 hinge); ERKCCVECPPCP (SEQ ID NO:123) (human IgG2 hinge); ELKTPLGDTTHTCPRCP (SEQ ID NO:124) (human IgG3 hinge); SPNMVPHAHHAQ (SEQ ID NO:125) (human IgG4 hinge); and the like.

The hinge region can comprise an amino acid sequence of a human IgG1, IgG2, IgG3, or IgG4, hinge region. The hinge region can include one or more amino acid substitutions and/or insertions and/or deletions compared to a wild-type (naturally-occurring) hinge region. For example, His₂₂₉ of human IgG1 hinge can be substituted with Tyr, so that the hinge region comprises the sequence EPKSCDKTYTCPPCP (SEQ ID NO:126); see, e.g., Yan et al. (2012) J. Biol. Chem. 287:5891.

The hinge region can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:127), or a variant thereof.

### Engineered T Cell Receptor (TCR)

In some instances, a heteromeric, conditionally repressible synthetic ICR may include, in part or in whole, an engineered T cell receptor (TCR) or may essentially be a modified engineered TCR such that by modification the engineered TCR is conditionally repressible. In such instances, the engineered TCR containing heteromeric, conditionally repressible synthetic ICR may be referred to as a heteromeric, conditionally repressible synthetic TCR or, for simplicity, a repressible TCR.

Any engineered TCR having immune cell activation function may find use in a heteromeric, conditionally repressible synthetic ICR as described herein including but not limited to, e.g., antigen-specific TCRs, Monoclonal TCRs (MTCRs), Single chain MTCRs, High Affinity CDR2 Mutant TCRs, CD1-binding MTCRs, High Affinity NY-ESO TCRs, VYG HLA-A24 Telomerase TCRs, including e.g., those described in PCT Pub Nos. WO 2003/020763, WO 2004/033685, WO 2004/044004, WO 2005/114215, WO 2006/000830, WO 2008/038002, WO 2008/039818, WO 2004/074322, WO 2005/113595, WO 2006/125962; Strommes et al. Immunol Rev. 2014; 257(1):145-64; Schmitt et al. Blood. 2013; 122(3):348-56; Chapuls et al. Sci Transl Med. 2013; 5(174):174ra27; Thaxton et al. Hum Vaccin Immunother. 2014; 10(11):3313-21 (PMID:25483644); Gschweng et al. Immunol Rev. 2014; 257(1):237-49 (PMID:24329801); Hinrichs et al. Immunol Rev. 2014; 257(1):56-71 (PMID:24329789); Zoete et al. Front Immunol. 2013; 4:268 (PMID:24062738); Marr et al. Clin Exp Immunol. 2012; 167(2):216-25 (PMID:22235997); Zhang et al. Adv Drug Deliv Rev. 2012; 64(8):756-62 (PMID:22178904); Chhabra et al. Scientific World Journal. 2011; 11:121-9 (PMID:21218269); Boulter et al. Clin Exp Immunol. 2005; 142(3):454-60 (PMID:16297157); Sami et al. Protein Eng Des Sel. 2007; 20(8):397-403; Boulter et al. Protein Eng. 2003; 16(9):707-11; Ashfield et al. IDrugs. 2006; 9(8):554-9; Li et al. Nat Biotechnol. 2005; 23(3):349-54; Dunn et al. Protein Sci. 2006; 15(4):710-21; Liddy et al. Mol Biotechnol. 2010; 45(2); Liddy et al. Nat Med. 2012; 18(6):980-7; Oates, et al. Oncoimmunology. 2013; 2(2):e22891; McCormack, et al. Cancer Immunol Immunother. 2013 April; 62(4):773-85; Bossi et al. Cancer Immunol Immunother. 2014; 63(5):437-48 and Oates, et al. Mol Immunol. 2015 October; 67(2 Pt A):67-74; van Hall et al. Nature Medicine. 2006; 12(4): 417-24; Doorduijn et al. J Clin Invest. 2016; 126(2): 784-94; Wang et al. J Immunol. 2008; 181(6): 3769-76; the disclosures of which are incorporated herein by reference in their entirety.

In some instances, an engineered TCR useful in a heteromeric, conditionally repressible synthetic ICR as described herein may include, e.g., a NY-ESO-1-binding TCR or a TCR that binds to NY-ESO-1 or a peptide derived therefrom. For example, in some instances a NY-ESO-1-binding TCR may be an engineered TCR that binds to a peptide having the amino acid sequence: SLLMWITQC (SEQ ID NO:128).

In some instances, an engineered TCR useful in a heteromeric, conditionally repressible synthetic ICR as described herein may be or may be derived from an engineered TCR having high affinity for its ligand including but not limited to, e.g., a K_{D} of less than or equal to 100 μM, including but not limited to e.g., a K_{D} of less than or equal to 10 μM or a K_{D} of less than or equal to 1 μM. In some instances, an engineered TCR useful in a heteromeric, conditionally repressible synthetic ICR as described herein may be or may be derived from an engineered TCR having high affinity for the peptide SLLMWITQC (SEQ ID NO:128), including but not limited to, e.g., a K_{D} of less than or equal to 100 μM, including but not limited to e.g., a K_{D} of less than or equal to 10 μM or a K_{D} of less than or equal to 1 μM for the peptide SLLMWITQC (SEQ ID NO:128). The K_{D} measurement can be made by any known method, including but not limited to e.g., Surface Plasmon Resonance (Biacore).

In some instances, an engineered TCR useful in a heteromeric, conditionally repressible synthetic ICR as described herein may be or may be derived from an engineered TCR having a slow off-rate (k_{off}) from its ligand including but not limited to, e.g., a k_{off} of 0.1 S or slower, including but not limited to e.g., a k_{off} of 1×10⁻² S⁻¹ or slower or a k_{off} of 1×10⁻³ S⁻¹ or slower. In some instances, an engineered TCR useful in a heteromeric, conditionally repressible synthetic ICR as described herein may be or may be derived from an engineered TCR having a slow off-rate from the peptide SLLMWITQC (SEQ ID NO:128), including but not limited to, e.g., a K_{D} of less than or equal to 100 μM, including but not limited to e.g., a k_{off} of 0.1 S⁻¹ or slower, including but not limited to e.g., a k_{off} of 1×10⁻² S⁻¹ or slower or a k_{off} of 1×10⁻³ S⁻¹ or slower from the peptide SLLMWITQC (SEQ ID NO:128). The k_{off} measurement can be made by any known method, including but not limited to e.g., Surface Plasmon Resonance (Biacore).

In some instances, an engineered TCR may be modified for use as a component of a heteromeric, conditionally repressible synthetic ICR through introduction or insertion of a dimerization domain (e.g., a member of a dimerizer pair) into the engineered TCR and, in such instances, following modification, the engineered TCR may be referred to as a dimerizer-domain containing TCR or a dimerizable TCR.

A dimerizer domain may be inserted into the engineered TCR amino acid sequence, e.g., by introducing a coding sequence for the dimerizer domain into the coding sequence of the engineered TCR, at any convenient location provided the insertion does not negatively impact the primary functional domains of the engineered TCR (including e.g., a TCR alpha chain domain, a TCR beta chain domain, a TCR CD3 chain domain, a TCR zeta chain domain, a TCR CD3-zeta chain domain a TCR extracellular domain, a TCR intracellular domain, a TCR variable region domain, a TCR constant region domain, a TCR IgSF domain, etc., or a function thereof) and/or the negatively impact the dimerization function of the dimerizer domain.

An engineered TCR may include one or more epsilon, sigma, or gamma chains, or in some instances, an engineered TCR may not include one or more epsilon, sigma, or gamma chains and may instead rely upon endogenously expressed epsilon, sigma, or gamma chains. In some instances, an engineered TCR may not include one or more CD3-zeta chains and may instead rely on endogenously expressed CD3-zeta.

In some instances, the dimerizer domain may be inserted into an extracellular portion of the engineered TCR. In some instances the dimerizer domain may be inserted into an intracellular portion of the engineered TCR.

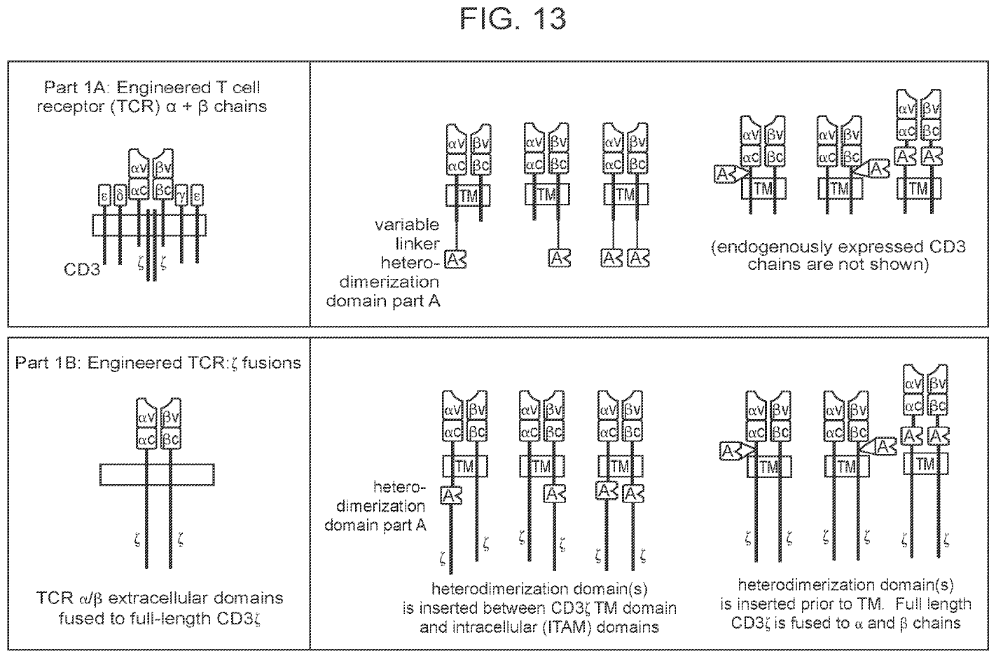

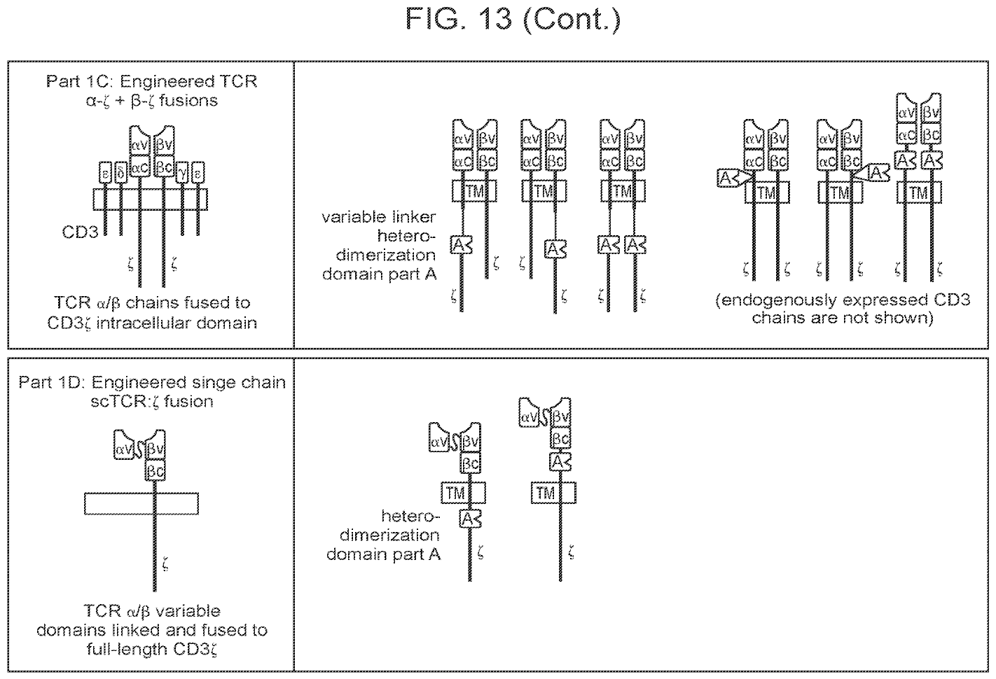

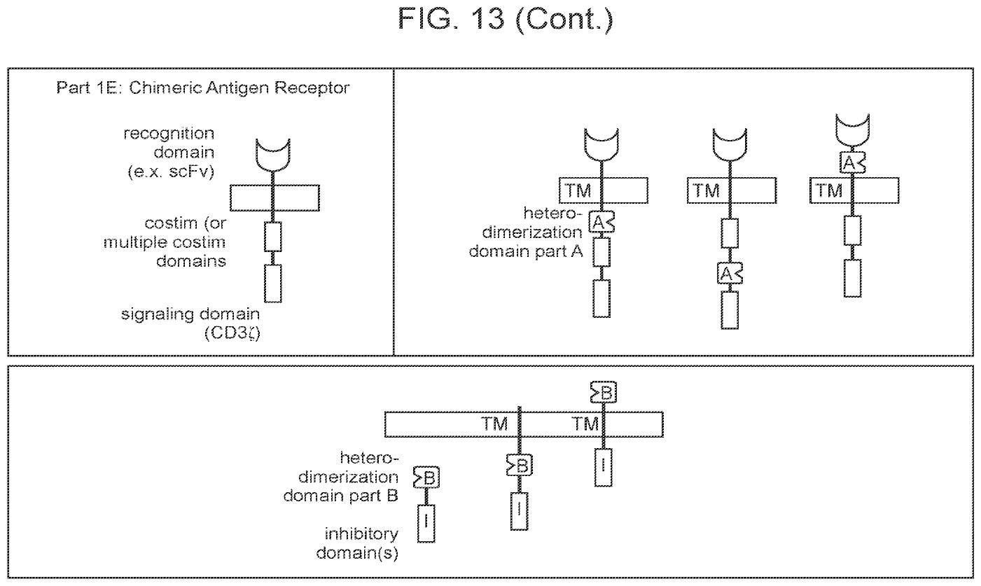

In some instances, the dimerizer domain may be inserted into or linked to an alpha chain of the engineered TCR. In some instances, the dimerizer domain is inserted or linked such that following the insertion or linking the dimerizer domain is linked intracellularly to the alpha chain including e.g., where the dimerizer domain is linked to the cytoplasmic side of the alpha chain transmembrane domain (see e.g., FIG. 13, Part 1A). In some instances, the dimerizer domain is inserted or linked such that following the insertion or linking the dimerizer domain is linked extracellularly to the alpha chain including e.g., where the dimerizer domain is linked to the extracellular side of the alpha chain transmembrane domain, where the dimerizer domain is inserted between the alpha chain transmembrane domain and the alpha chain constant region domain, etc. (see e.g., FIG. 13, Part 1A).

In some instances, the dimerizer domain may be inserted into or linked to a beta chain of the engineered TCR. In some instances, the dimerizer domain is inserted or linked such that following the insertion or linking the dimerizer domain is linked intracellularly to the beta chain including e.g., where the dimerizer domain is linked to the cytoplasmic side of the beta chain transmembrane domain (see e.g., FIG. 13, Part 1A). In some instances, the dimerizer domain is inserted or linked such that following the insertion or linking the dimerizer domain is linked extracellularly to the beta chain including e.g., where the dimerizer domain is linked to the extracellular side of the beta chain transmembrane domain, where the dimerizer domain is inserted between the beta chain transmembrane domain and the beta chain constant region domain, etc. (see e.g., FIG. 13, Part 1A).

In some instances, the dimerizer domain may be inserted into or linked to a fused alpha-CD3-zeta chain, e.g., where the CD3-zeta chain is full-length CD3-zeta (e.g., a TCR:zeta fusion) of the engineered TCR. In some instances, the dimerizer domain is inserted or linked such that following the insertion or linking the dimerizer domain is linked intracellularly to the fused alpha-CD3-zeta chain including e.g., where the dimerizer domain is inserted between the CD3-zeta transmembrane domain and other intracellular domains of the fused alpha-CD3-zeta chain, including e.g., one or more intracellular ITAM domains in (see e.g., FIG. 13, Part 1B). In some instances, the dimerizer domain is inserted or linked such that following the insertion or linking the dimerizer domain is linked extracellularly to the fused alpha-CD3-zeta chain including e.g., where the dimerizer domain is linked to the extracellular side of the CD3-zeta transmembrane domain, where the dimerizer domain is inserted between the extracellular alpha chain domain and the transmembrane domain of the fused CD3-zeta, etc. (see e.g., FIG. 13, Part 1B).

In some instances, the dimerizer domain may be inserted into or linked to a fused beta-CD3-zeta chain, e.g., where the CD3-zeta chain is full-length CD3-zeta (e.g., a TCR:zeta fusion) of the engineered TCR. In some instances, the dimerizer domain is inserted or linked such that following the insertion or linking the dimerizer domain is linked intracellularly to the fused beta-CD3-zeta chain including e.g., where the dimerizer domain is inserted between the CD3-zeta transmembrane domain and other intracellular domains of the fused beta-CD3-zeta chain, including e.g., one or more intracellular ITAM domains in (see e.g., FIG. 13, Part 1B). In some instances, the dimerizer domain is inserted or linked such that following the insertion or linking the dimerizer domain is linked extracellularly to the fused beta-CD3-zeta chain including e.g., where the dimerizer domain is linked to the extracellular side of the CD3-zeta transmembrane domain, where the dimerizer domain is inserted between the extracellular beta chain domain and the transmembrane domain of the fused CD3-zeta, etc. (see e.g., FIG. 13, Part 1B).

In some instances, the dimerizer domain may be inserted into or linked to a fused alpha-CD3-zeta domain (e.g., in an engineered TCR alpha-zeta+beta-zeta fusion) of the engineered TCR. In some instances, the dimerizer domain is inserted or linked such that following the insertion or linking the dimerizer domain is linked intracellularly to the fused alpha-CD3-zeta domain including e.g., where the dimerizer domain is inserted between one or more domains of the CD3-zeta domain and the transmembrane domain of the alpha chain (see e.g., FIG. 13, Part 1C). In some instances, the dimerizer domain is inserted or linked such that following the insertion or linking the dimerizer domain is linked extracellularly to the fused alpha-CD3-zeta domain including e.g., where the dimerizer domain is linked to the extracellular side of the alpha chain transmembrane domain, where the dimerizer domain is inserted between the alpha chain transmembrane domain and the alpha chain constant region domain, etc. (see e.g., FIG. 13, Part 1C).

In some instances, the dimerizer domain may be inserted into or linked to a fused beta-CD3-zeta domain (e.g., in an engineered TCR alpha-zeta+beta-zeta fusion) of the engineered TCR. In some instances, the dimerizer domain is inserted or linked such that following the insertion or linking the dimerizer domain is linked intracellularly to the fused beta-CD3-zeta domain including e.g., where the dimerizer domain is inserted between one or more domains of the CD3-zeta domain and the transmembrane domain of the beta chain (see e.g., FIG. 13, Part 1C). In some instances, the dimerizer domain is inserted or linked such that following the insertion or linking the dimerizer domain is linked extracellularly to the fused beta-CD3-zeta domain including e.g., where the dimerizer domain is linked to the extracellular side of the beta chain transmembrane domain, where the dimerizer domain is inserted between the beta chain transmembrane domain and the beta chain constant region domain, etc. (see e.g., FIG. 13, Part 1C).

In some instances, the dimerizer domain may be inserted into or linked to a chain of an engineered single chain TCR (e.g., in an engineered single chain TCR:zeta fusion, e.g., where a TCR alpha chain variable domain is linked to a TCR beta chain which is fused to a full-length CD3-zeta chain). In some instances, the dimerizer domain is inserted or linked such that following the insertion or linking the dimerizer domain is linked intracellularly to the engineered single chain TCR including e.g., where the dimerizer domain is inserted between one or more domains of the CD3-zeta chain and the transmembrane domain of the CD3-zeta chain (see e.g., FIG. 13, Part 1D). In some instances, the dimerizer domain is inserted or linked such that following the insertion or linking the dimerizer domain is linked extracellularly to the engineered single chain TCR including e.g., where the dimerizer domain is linked to the extracellular side of the CD3-zeta chain transmembrane domain, where the dimerizer domain is inserted between the CD3-zeta chain transmembrane domain and the beta chain constant region domain, etc. (see e.g., FIG. 13, Part 1D).

In some instances, only a single dimerizer domain may be present in a conditionally repressible engineered TCR, e.g., where a single dimerizer domain is linked or inserted into an alpha chain of the engineered TCR, where a single dimerizer domain is linked or inserted into a beta chain of the engineered TCR, where a single dimerizer domain is linked or inserted into a CD3-zeta chain of the engineered TCR, etc. For non-limiting examples, see FIG. 13, Parts 1A-1D.

In some instances, two or more dimerizer domains may be present in a conditionally repressible engineered TCR. For example, two dimerizer domains may be present in a conditionally repressible engineered TCR, e.g., where a first dimerizer domain is linked or inserted into an alpha chain of the engineered TCR and a second dimerizer domain is linked or inserted into a beta chain of the engineered TCR, where a first dimerizer domain is linked or inserted into a first CD3-zeta chain of the engineered TCR and a second dimerizer domain is linked or inserted into a second CD3-zeta chain of the engineered TCR, etc. For non-limiting examples, see FIG. 13, Parts 1A-1C.

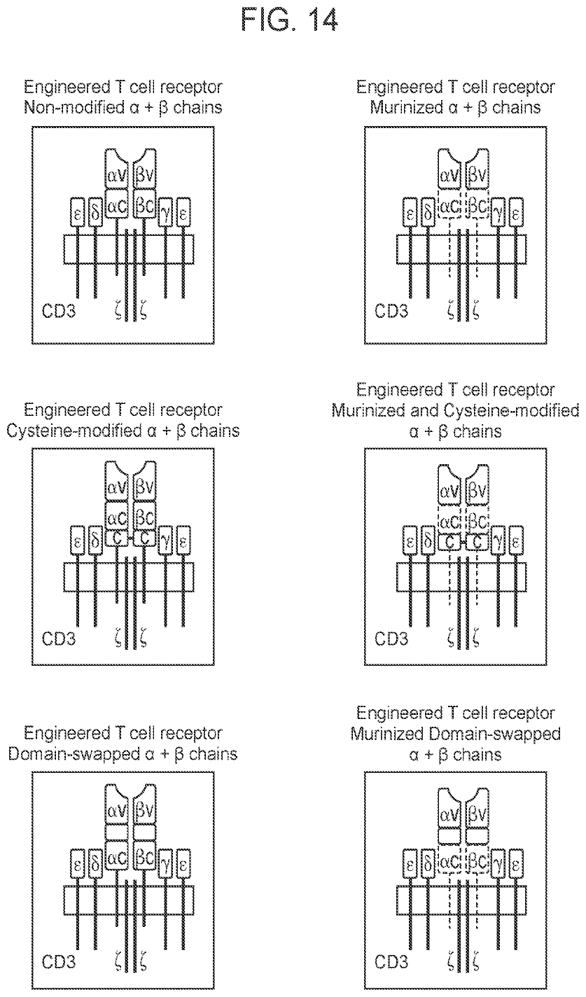

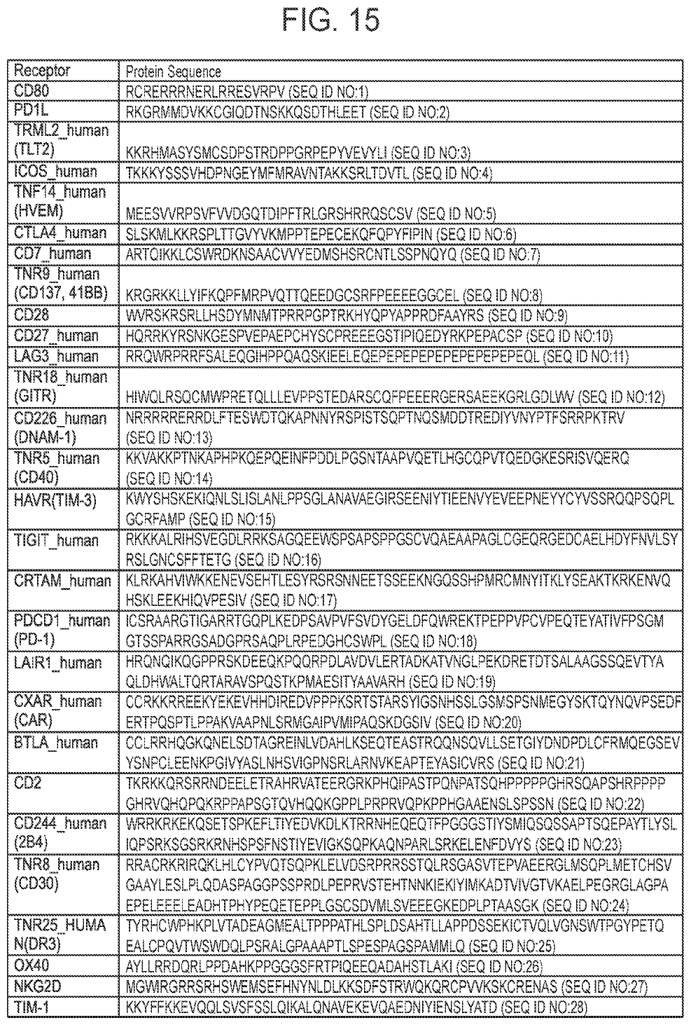

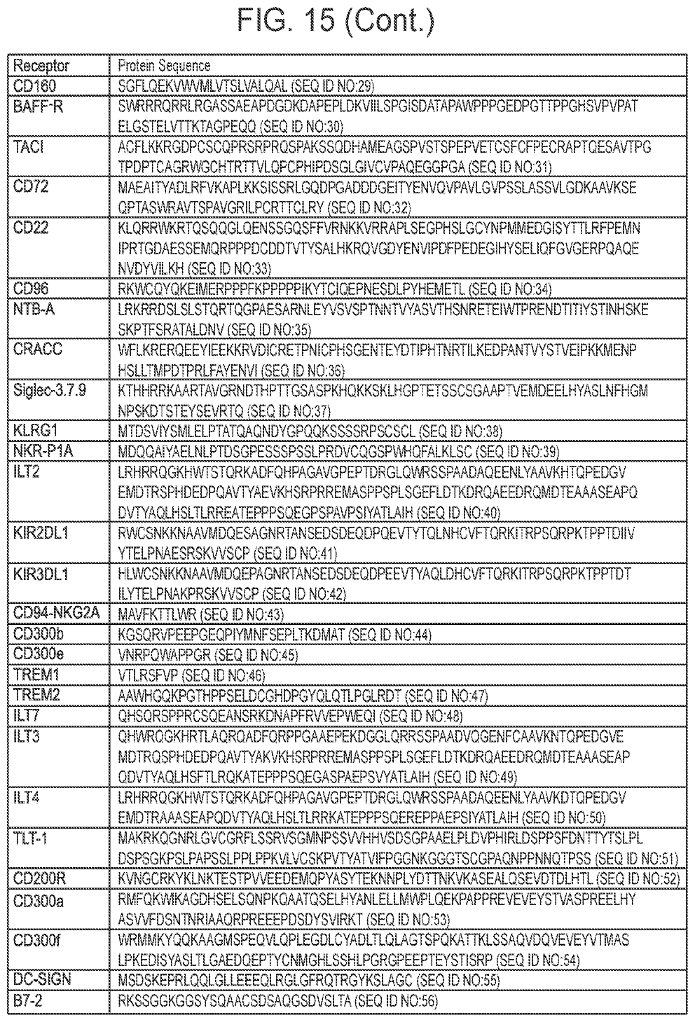

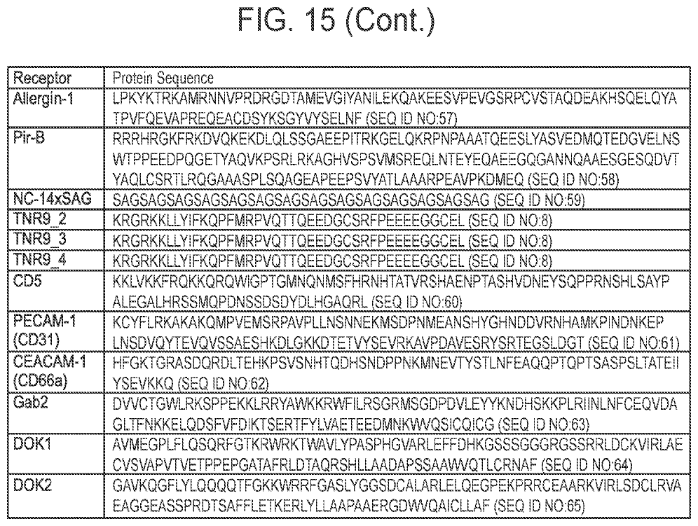

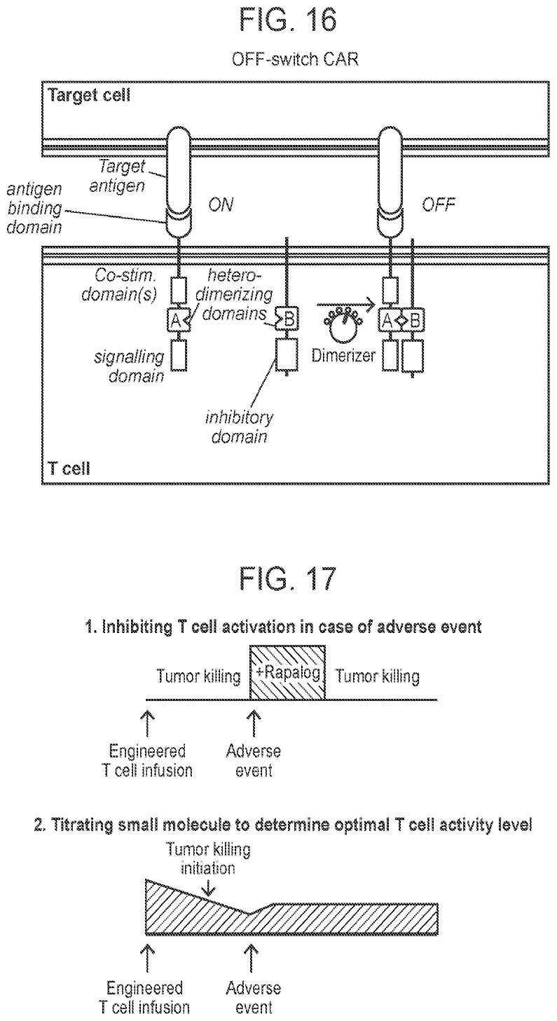

In some instances, the engineered TCR of a conditionally repressible TCR may be an engineered TCR variant including but not limited to, e.g., those engineered TCR variants depicted in FIG. 14, e.g., including engineered TCR variants that include one or more variant or mutant TCR chains. In some instances, the engineered TCR of a conditionally repressible TCR may include one or more non-modified chains, including but not limited to a non-modified alpha chain, a non-modified beta chain, etc. In some instances, the engineered TCR of a conditionally repressible TCR may include one or more murinized chains, including but not limited to, e.g., a murinized alpha chain, a murinized beta chain, etc. In some instances, the engineered TCR of a conditionally repressible TCR may include one or more cysteine modified chains, including but not limited to, e.g., a cysteine modified alpha chain, a cysteine modified beta chain, etc. In some instances, the engineered TCR of a conditionally repressible TCR may include one or more domain-swapped chains, including but not limited to, e.g., a domain-swapped alpha chain, a domain swapped beta chain, etc.

In some instances, the engineered TCR of a conditionally repressible TCR may include one or more domain-swapped chains. By “domain-swapped chains” is generally meant TCR chains in which constant domains have been swapped between the a and chains. When paired, domain-swapped TCRs assemble with CD3, express on the cell surface, and mediate antigen-specific T cell responses. Useful examples of domain-swapped chains include but are not limited to e.g., those described in Bethune et al. eLife 2016; 5:e19095; the disclosure of which is incorporated herein by reference in its entirety. In some instances, the engineered TCR of a conditionally repressible TCR may include a domain-swapped alpha chain, a domain-swapped beta chain, and/or the like.

In some instances, the engineered TCR of a conditionally repressible TCR may include a combination of variant TCR chains, including but not limited to a combination of murinized, cysteine-modified, and domain-swapped chains, including but not limited to, e.g., a murinized and cysteine-modified alpha chain, a murinized and cysteine-modified beta chain, a murinized alpha chain and cysteine-modified beta chain, a murinized beta chain and cysteine-modified alpha chain, a murinized and domain-swapped alpha chain, a murinized and domain-swapped beta chain, etc.

In instances where a heteromeric, conditionally repressible synthetic ICR includes, in part or in whole, or the heteromeric, conditionally repressible synthetic ICR is essentially a modified TCR, the TCR may contain non-modified TCR chains having extracellular domains or the extracellular domains therefore present in modified TCR chains, one or more intracellular stimulatory domains present in non-modified or modified TCR chains and the transmembrane domains of such extracellular domain-containing or intracellular domain-containing chains. Such a TCR may optionally include linker regions and/or hinge regions. TCRs as part of a heteromeric, conditionally repressible synthetic ICR may be encompassed within a single polypeptide (e.g., as in engineered single chain TCRs) or various chains and portions thereof may be “split” across two or more polypeptides.

### TCR Chains

Many native TCRs exist in heterodimeric αβ or γδ forms. However, recombinant or engineered TCR may include a single TCR α or TCR β chain and may bind to peptide MHC molecules. In certain embodiments, an engineered TCR of a repressible ICR includes both an a chain variable domain and an TCR β chain variable domain. The chains of an engineered TCR useful in a repressible ICR of the instant disclosure may vary and may include any suitable native or synthetic or recombinant or mutant TCR chain or chains or combination thereof.

As will be obvious to those skilled in the art the mutation(s) in TCR chain sequence, including e.g., a chain sequence and/or TCR β chain sequence, may be one or more of substitution(s), deletion(s) or insertion(s). These mutations can be carried out using any appropriate method including, but not limited to, those based on polymerase chain reaction (PCR), restriction enzyme-based cloning, or ligation independent cloning (LIC) procedures. These methods are detailed in many standard molecular biology texts, including but not limited to e.g., Sambrook & Russell, (2001) Molecular Cloning—A Laboratory Manual (3^{rd} Ed.) CSHL Press and Rashtchian, (1995) Curr Opin Biotechnol 6 (1): 30-6.

As used herein the term “variable domain” is understood to encompass all amino acids of a given TCR which are not included within the constant domain as encoded by the TRAC gene for TCR α chains and either the TRBC1 or TRBC2 for TCR β chains as described in, e.g., T cell receptor Factsbook, (2001) LeFranc and LeFranc, Academic Press.

In some instances, an engineered TCR has at least one TCR α chain domain having or derived from an amino acid sequence that is at least 70% identical, including at least 75% identical to, including at least 80% identical to, including at least 85% identical to, including at least 90% identical to, including at least 95% identical to or is 100% identical to the IG4 α chain amino acid sequence:

(SEQ ID NO: 129)
METLLGLLILWLQLQWVSSKQEVTQIPAALSVPEGENLVLNCSFTDSAIY
NLQWFRQDPGKGLTSLLLIQSSQREQTSGRLNASLDKSSGRSTLYIAASQ
PGDSATYLCAVRPTSGGSYIPTFGRGTSLIVHPPNIQNPDPAVYQLRDSK
SSDKSVCLFTDFDSQTNVSQSKDSDVYITDKTVLDMRSMDFKSNSAVAWS
NKSDFACANAFNNSIIPEDTFFPSPESSCDVKLVEKSFETDTNLNFQNLS
VIGFRILLLKVAGFNLLMTLRLWSS.

In some instances, an engineered TCR has at least one TCR α chain domain having or derived from an amino acid sequence that is at least 70% identical, including at least 75% identical to, including at least 80% identical to, including at least 85% identical to, including at least 90% identical to, including at least 95% identical to or is 100% identical to the IG4 α chain A95:LY mutant amino acid sequence:

(SEQ ID NO: 130)
METLLGLLILWLQLQWVSSKQEVTQIPAALSVPEGENLVLNCSFTDSAIY
NLQWFRQDPGKGLTSLLLIQSSQREQTSGRLNASLDKSSGRSTLYIAASQ
PGDSATYLCAVRPLYGGSYIPTFGRGTSLIVHPPNIQNPDPAVYQLRDSK
SSDKSVCLFTDFDSQTNVSQSKDSDVYITDKTVLDMRSMDFKSNSAVAWS
NKSDFACANAFNNSIIPEDTFFPSPESSCDVKLVEKSFETDTNLNFQNLS
VIGFRILLLKVAGFNLLMTLRLWSS.

In some instances, an engineered TCR has at least one TCR β chain domain having or derived from an amino acid sequence that is at least 70% identical, including at least 75% identical to, including at least 80% identical to, including at least 85% identical to, including at least 90% identical to, including at least 95% identical to or is 100% identical to the IG4 β chain amino acid sequence:

(SEQ ID NO: 131)
MSIGLLCCAALSLLWAGPVNAGVTQTPKFQVLKTGQSMTLQCAQDMNHEY
MSWYRQDPGMGLRLIHYSVGAGITDQGEVPNGYNVSRSTTEDFPLRLLSA
APSQTSVYFCASSYVGNTGELFFGEGSRLTVLEDLNKVFPPEVAVFEPSE
AEISHTQKATLVCLATGFFPDHVELSWWVNGKEVHSGVSTDPQPLKEQPA
LNDSRYCLSSRLRVSATFWQNPRNHFRCQVQFYGLSENDEWTQDRAKPVT
QIVSAEAWGRADCGFTSVSYQQGVLSATILYEILLGKATLYAVLVSALVL
MAMVKRKDF.

In some instances, an engineered TCR has at least one TCR β chain domain having or derived from an amino acid sequence that is at least 70% identical, including at least 75% identical to, including at least 80% identical to, including at least 85% identical to, including at least 90% identical to, including at least 95% identical to or is 100% identical to the IG4 β chain G51A mutant amino acid sequence:

(SEQ ID NO: 132)
MSIGLLCCAALSLLWAGPVNAGVTQTPKFQVLKTGQSMTLQCAQDMNHEY
MSWYRQDPGMGLRLIHYSVAAGITDQGEVPNGYNVSRSTTEDFPLRLLSA
APSQTSVYFCASSYVGNTGELFFGEGSRLTVLEDLNKVFPPEVAVFEPSE
AEISHTQKATLVCLATGFFPDHVELSWWVNGKEVHSGVSTDPQPLKEQPA
LNDSRYCLSSRLRVSATFWQNPRNHFRCQVQFYGLSENDEWTQDRAKPVT
QIVSAEAWGRADCGFTSVSYQQGVLSATILYEILLGKATLYAVLVSALVL
MAMVKRKDF.

In some instances, a NY-ESO-1-binding TCR has at least one TCR α chain variable domain having an amino acid sequence that is at least 70% identical, including at least 75% identical to, including at least 80% identical to, including at least 85% identical to, including at least 90% identical to, including at least 95% identical to or is 100% identical to the a chain extracellular sequence: MQEVTQIPAALSVPEGENLVLNCSFTDSAIYNLQWFRQDPGKGLTSLLLIQSSQREQTSG RLNASLDKSSGRSTLYIAASQPGDSATYLCAVRPTSGGSYIPTFGRGTSLIVHPYIQNPDP AVYQLRDSKSSDKSVCLFTDFDSQTNVSQSKDSDVYITDKTVLDMRSMDFKSNSAVA WSNKSDFACANAFNNSIIPEDTFFPSPESS (SEQ ID NO:133). In some instances, the a chain extracellular sequence contains one or more of the following amino acid substitutions: T95L and S96Y.

In some instances, a NY-ESO-1-binding TCR has at least one TCR β chain variable domain having an amino acid sequence that is at least 70% identical, including at least 75% identical to, including at least 80% identical to, including at least 85% identical to, including at least 90% identical to, including at least 95% identical to or is 100% identical to the β chain extracellular sequence:

(SEQ ID NO: 134)
MGVTQTPKFQVLKTGQSMTLQCAQDMNHEYMSWYRQDPGMGLRLIHYSVG
AGITDQGEVPNGYNVSRSTTEDFPLRLLSAAPSQTSVYFCASSYVGNTGE
LFFGEGSRLTVLEDLKNVFPPEVAVFEPSEAEISHTQKATLVCLATGFYP
DHVELSWWVNGKEVHSGVSTDPQPLKEQPALNDSRYALSSRLRVSATFWQ
DPRNHFRCQVQFYGLSENDEWTQDRAKPVTQIVSAEAWGRAD.

In some instances, the engineered TCR include an introduced disulfide bond between cysteines. For example, disulfide bond between cysteines may be introduced between substitute amino acids of two chains of the engineered including but not limited to, e.g., between an a chain and a β chain of the TCR. In some instances an engineered TCR may be a NY-ESO-1-binding TCR that includes a disulfide bond between cysteines of substitute amino acids of two chains of the engineered NY-ESO-1-binding TCR including but not limited to, e.g., between an a chain and a β chain of the engineered NY-ESO-1-binding TCR. For example, in some instances, an engineered NY-ESO-1-binding TCR may include a disulfide bond between cysteines substituted for alpha chain T162 and beta chain S169 of SEQ ID NOs:133-134.

### Linkers

In some cases, a subject conditionally repressible TCR includes a linker between any two adjacent domains or artificially linked chains. For example, a linker can be disposed between the intracellular potion of a transmembrane domain of an alpha chain and a dimerizer domain of the conditionally repressible TCR. In some instances, a linker can be disposed between the intracellular potion of a transmembrane domain of a beta chain and a dimerizer domain of the conditionally repressible TCR. In some instances, a linker can be disposed between the transmembrane domain of an alpha chain and the first intracellular functional domain of a linked CD3-zeta chain of the conditionally repressible TCR. In some instances, a linker can be disposed between the transmembrane domain of a beta chain and the first intracellular functional domain of a linked CD3-zeta chain of the conditionally repressible TCR. As another example, a linker can be disposed between any domain of the conditionally repressible TCR and any additional domain including e.g., a domain not involved in the primary immune activation functions of the conditionally repressible TCR including but not limited to e.g., a reporter domain, a tag domain, etc.

Linkers may be utilized in a suitable configuration in the conditionally repressible TCR provided they do not abolish the primary activities of the conditionally repressible TCR including, e.g., the ability of the conditionally repressible TCR to activate an immune cell, the ability of the dimerization domain of the conditionally repressible TCR to bind the dimerization domain of the synthetic ICR repressor, etc.

Any suitable linker, including two or more linkers (e.g., where the two or more linkers are the same or different and including where the multiple linkers are three or more, four or more, five or more, six or more, etc. and including where all the linkers are different and where the multiple linkers include an mix of some linkers utilized in more than one location and some linkers utilized specifically in only one location and the like) may be utilized in the subject conditionally repressible TCRs including e.g., those linkers described herein for acceptable use in a CAR.

### T Cell-Antigen Coupler (TAC)

In some instances, a heteromeric, conditionally repressible synthetic ICR may include, in part or in whole, a T cell-antigen coupler (TAC) or may essentially be a modified TAC (examples of which include a Trifunctional T Cell-Antigen Coupler or “Tri-TAC”) such that by modification the TAC is conditionally repressible. In such instances, the TAC-containing heteromeric, conditionally repressible synthetic ICR may be referred to as a heteromeric, conditionally repressible TAC or, for simplicity, a repressible TAC.

In some instances, a TAC may be modified for use as a component of a heteromeric, conditionally repressible synthetic ICR through introduction or insertion of a dimerization domain (e.g., a member of a dimerizer pair) into the TAC and, in such instances, following modification, the TAC may be referred to as a dimerizer-domain containing TAC or a dimerizable TAC.

A dimerizable TAC will generally include a TCR specific binding domain, a transmembrane domain, an intracellular signaling domain and one member of a dimerizer-binding pair. In some instances, a TAC will further include a target specific binding domain. When bound to the member of the TCR to which the TCR specific binding domain binds (and the target of the target-specific binding domain where applicable), the dimerizable TAC becomes active (e.g., activating a cell in which it is expressed). Such a dimerizable TAC may be utilized as a synthetic stimulatory ICR that, in the presence of the dimerizer, dimerizes with a corresponding second member of the dimerizer-binding pair present in a synthetic ICR repressor to repress the activity of the TAC. Dimerizing the dimerizable TAC with a corresponding second member of the dimerizer-binding pair present in a synthetic ICR repressor may also be used to repress the activity of the TCR to which the TAC is bound.

A dimerizer domain may be inserted into the TAC amino acid sequence, e.g., by introducing a coding sequence for the dimerizer domain into the coding sequence of the TAC, at any convenient location provided the insertion does not negatively impact the primary functional domains of the TAC (including e.g., the target-specific binding domain, the TCR specific binding domain, the intracellular signaling domain, etc.) and/or negatively impact the dimerization function of the dimerizer domain.

The target-specific binding domain of a TAC, where present, generally directs the TAC to a target molecule (e.g., a cell expressing the target molecule) through specific binding of a target molecule. Any convenient specific binding pair may find use as a target-specific binding member and target molecule of a TAC, including but not limited to e.g., members of the extracellular recognition domains described herein. Upon binding the target of the target-specific binding domain, the TAC (also bound to the protein associated with the TCR complex to which the TCR specific binding domain binds) facilitates signaling through the intracellular signaling domain to stimulate activity of the cell expressing the TAC.

In some instances, the target-specific binding domain of a TAC is an antigen binding domain. In some instances, the antigen to which the target-specific binding domain of a TAC binds is a tumor antigen. Useful tumor antigens (e.g. which can be represented by MHC complexes) will vary and may be, e.g., a sequence of 8 or more amino acids up to the full protein and any number of amino acids in between 8 and the full length protein which includes at least one antigenic fragment of the full length protein that can be represented in a MHC complex. Non-limiting examples of tumor antigens include but are not limited to e.g., HER2 (erbB-2), B-cell maturation antigen (BCMA), alphafetoprotein (AFP), carcinoembryonic antigen (CEA), CA-125, MUC-1, epithelial tumor antigen (ETA), tyrosinase, melanoma-associated antigen (MAGE), prostate-specific antigen (PSA), glioma-associated antigen, β-human chorionic gonadotropin, thyroglobulin, RAGE-1, MN-CA IX, human telomerase reverse transcriptase, RU1, RU2 (AS), intestinal carboxyl esterase, mut hsp70-2, M-CSF, prostase, PAP, NY-ESO-1, LAGE-1 a, p53, prostein, PSMA, survivin and telomerase, prostate-carcinoma tumor antigen-1 (PCTA-1), ELF2M, neutrophil elastase, CD22, insulin growth factor (IGF)-I, IGF-II, IGF-I receptor, mesothelin and the like.

Non-limiting examples of useful target-specific binding domains include but are not limited to e.g., antibodies and fragments thereof, for example single chain antibodies such as scFVs, small proteins that bind to the target cell and/or antigen, and the like. In some instances, a target-specific binding domain of a TAC may be a designed ankyrin repeat (DARPin) targeted to a specific cell and/or antigen, including but not limited to e.g., HER2.

The TCR specific binding domain binds a protein associated with the T-cell receptor complex and generally serves to recruit the T-Cell Receptor (TCR) in combination with co-receptor stimulation. Accordingly, a TCR specific binding domain includes any substance that binds, directly or indirectly, to a protein of the TCR. Proteins associated with the TCR include, but are not limited to the TCR alpha (α) chain, TCR beta (β) chain, TCR gamma (γ) chain, TCR delta (δ) chain, CD3γ chain, CD3δ chain and CD3ε chains. In some instances, a TCR specific binding domain that binds to a protein associated with the T-cell receptor complex is an antibody to the TCR alpha (α) chain, TCR beta (β) chain, TCR gamma (γ) chain, TCR delta (δ) chain, CD3γ chain, CD3δ chain and/or CD3ε chain.

In some instances, a TCR specific binding domain of a subject TAC may be an antibody or a fragment thereof that binds CD3. Examples of CD3 antibodies include but are not limited to e.g., muromonab, otelixizumab, teplizumab, visilizumab, UCHT1 (which targets CD3E), and the like. In some instances, the antibody that binds CD3 is a single chain antibody, e.g., a single-chain variable fragment (scFv).

The intracellular signaling domain of a TAC may be any polypeptide that propagates extracellular binding of the TCR specific binding domain (and the target-specific binding domain where present) to an intracellular signal resulting in stimulation of the cell expressing the TAC. In some instances, the intracellular signaling domain is a T cell receptor signaling domain polypeptide that (a) localizes to the lipid raft and/or (b) binds lymphocyte-specific protein tyrosine kinase (Lck). In some instances, the intracellular signaling domain of a TAC includes one or more Lck interaction sites. In some instances, a intracellular signaling domain of a TAC includes one or more CD4-derived Lck interaction sites.

Intracellular signaling domains of a TAC may include e.g., one or more TCR co-receptors or domains thereof, one or more co-stimulators or domains thereof. In reference to the subject TACs, a “TCR co-receptor” refers to a molecule that assists the T cell receptor (TCR) in communicating with an antigen-presenting cell. Examples of TCR co-receptors useful in a TAC include, but are not limited to, CD4, CD8, CD28, CD45, CD5, CD9, CD16, CD22, CD33, CD37, CD64, CD80, CD86, CD134, and CD154. In reference to the subject TACs, a “TCR co-stimulator” refers to a molecule that is required for the response of a T cell to an antigen. Examples of TCR co-stimulators include, but are not limited to, PD-1, ICOS, CD27, CD28, 4-1 BB (CD137), OX40, CD30, CD40, lymphocyte function-associated antigen 1 (LFA-1), CD2, CD7, LIGHT, NKG2C, B7-H3, and a ligand that specifically binds CD83.

A subject TAC will also generally include a transmembrane domain. Useful transmembrane domains may include but are not limited to e.g., those described elsewhere herein, transmembrane domains of a TCR co-receptor or co-stimulator protein. In some instances, the intracellular signaling domain domain of a TAC and the transmembrane domain may be derived from the same co-receptor or co-stimulator or from different co-receptors or co-stimulators. In some instances, the intracellular signaling domain and the transmembrane domains are optionally joined by a linker (including but not limited to e.g., one or more linkers described herein).

In some instances, a subject dimerizable TAC includes a transmembrane domain of the CD4 co-receptor, an intracellular domain of the CD4 co-receptor or both. In some instances, a subject dimerizable TAC includes a transmembrane domain of the CD8 co-receptor, an intracellular domain of the CD8 co-receptor or both. In some instances, the intracellular signaling domain and/or transmembrane domain of the dimerizable TAC is synthetic, e.g., the transmembrane domain may be a synthetic, highly hydrophobic membrane domain. In some instances, the transmembrane domain is a glycophorine transmembrane domain. In some instances, the TAC includes a CD48 GPI signal sequence to attach the TAC to the membrane using the GPI anchor.

The domains of TAC polypeptides that may be rendered dimerizable by insertion or addition of a member of a dimerizer pair, may be configured in various arrangements and thus are not limited to those arrangements specifically disclosed. In one embodiment, the target-specific binding domain and the intracellular signaling domain, via the transmembrane domain, are both fused to the TCR specific binding domain.

Any TAC having immune cell activation function may find use in a heteromeric, conditionally repressible TAC as described herein including but not limited to, e.g., those TAC polypeptides (including e.g., N-DARPin TAC) and domains thereof described in PCT International Publication Number WO/2015/117229; the disclosure of which is incorporated herein by reference in its entirety. Useful TAC polypeptides and domains thereof further include those developed by Triumvira Immunologics Inc. (Hamilton, ON, Canada).

Useful TACs include e.g., N-Darpin Tri-TAC and C-Darpin Tri-TAC the amino acid sequences of which are, respectively:

(SEQ ID NO: 135)
MDFQVQIFSFLLISASVIMSRGSDLGKKLLEAARAGQDDEVRILMANGAD
VNAKDEYGLTPLYLATAHGHLEIVEVLLKNGADVNAVDAIGFTPLHLAAF
IGHLEIAEVLLKHGADVNAQDKFGKTAFDISIGNGNEDLAEILQKLNEQK
LISEEDLNPGGGGGSGGGGSGGGGSGGGGSGSMDIQMTQTTSSLSASLGD
RVTISCRASQDIRNYLNWYQQKPDGTVKLLIYYTSRLHSGVPSKFSGSGS
GTDYSLTISNLEQEDIATYFCQQGNTLPWTFAGGTKLEIKGGGGSGGGGS
GGGGSGGGGSEVQLQQSGPELVKPGASMKISCKASGYSFTGYTMNWVKQS
HGKNLEWMGLINPYKGVSTYNQKFKDKATLTVDKSSSTAYMELLSLTSED
SAVYYCARSGYYGDSDWYFDVWGQGTTLTVFSTSGGGGSLESGQVLLESN
IKVLPTWSTPVQPMALIVLGGVAGLLLFIGLGIFFCVRCRHRRRQAERMS
QIKRLLSEKKTCQCPHRFQKTCSPI
and
(SEQ ID NO: 136)
MDFQVQIFSFLLISASVIELGGGGSGSMDIQMTQTTSSLSASLGDRVTIS
CRASQDIRNYLNWYQQKPDGTVKLLIYYTSRLHSGVPSKFSGSGSGTDYS
LTISNLEQEDIATYFCQQGNTLPWTFAGGTKLEIKGGGGSGGGGSGGGGS
GGGGSEVQLQQSGPELVKPGASMKISCKASGYSFTGYTMNWVKQSHGKNL
EWMGLINPYKGVSTYNQKFKDKATLTVDKSSSTAYMELLSLTSEDSAVYY
CARSGYYGDSDWYFDVWGQGTTLTVFSTSGGGGSGGGGSGGGGSGGGGSD
IMSRGSDLGKKLLEAARAGQDDEVRILMANGADVNAKDEYGLTPLYLATA
HGHLEIVEVLLKNGADVNAVDAIGFTPLHLAAFIGHLEIAEVLLKHGADV
NAQDKFGKTAFDISIGNGNEDLAEILQKLNEQKLISEEDLNVDGGGGSGG
GGSGGGGSGGGGSLESGQVLLESNIKVLPTWSTPVQPMALIVLGGVAGLL
LFIGLGIFFCVRCRHRRRQAERMSQIKRLLSEKKTCQCPHRFQKTCSPI.

### Synthetic ICR Repressor

As described herein, a heteromeric, conditionally repressible synthetic ICR includes a synthetic ICR repressor, also referred to herein as an “ICR repressor” or “inhibitory part” for simplicity. Such inhibitory ICRs will vary depending on the particular context of immune cell repression to which the construct is directed and will generally function to mediate repression of an activated or activatable immune cell expressing a stimulatory ICR and the ICR repressor. Thus, an ICR repressor includes an inhibitory domain that functions to repress immune cell activation attributed to the stimulatory ICR upon dimerization of reciprocal dimerizer domains present in the ICR repressor and the stimulatory ICR when dimerizer is present.

A ICR repressor therefore includes one or more intracellular inhibitory domains that mediates intracellular signaling leading to inhibition of immune cell activation in immune cells expressing the stimulatory ICR. Domains useful as inhibitory domains will vary depending on the particular context of immune cell activation and repression, including e.g., the particular type of activated cell to be repressed and the desired degree of repression. Exemplary non-limited examples of inhibitory domains, described in greater detail below, include but are not limited to domains and motifs thereof derived from immune receptors including, e.g., co-inhibitory molecules, immune checkpoint molecules, immune tolerance molecules, and the like.

An ICR repressor further includes, as described in more detail below, a domain of a dimerization pair. Useful dimerization domains will vary depending on the desired dimerizer and the desired relative position of the dimerization domain within the ICR repressor. Generally, the presence of a first domain of a dimerization pair within the stimulatory ICR mediates the dimerization, upon introduction of the dimerizer, with a second domain of the dimerization pair present in the ICR repressor such that upon dimerization the ICR repressor represses immune cell activation due to the stimulatory ICR.

An ICR repressor may, optionally, include a transmembrane domain. As such, ICR repressors as described herein may or may not be membrane tethered. As such, an ICR repressor may contain a transmembrane domain, or portion thereof, and thus may be a membrane-bound ICR repressor. In other instances, an ICR repressor may lack a transmembrane domain and thus may be a cytosolic ICR repressor. Such transmembrane domains useful in an ICR repressor of the instant disclosure are described further herein.

In some instances, an ICR repressor may further include additional domains. Such additional domains may be functional, e.g., they directly contribute to the immune cell activation inhibition function of the ICR repressor, or non-functional, e.g., they do not directly contribute to the repression function of the ICR repressor. Non-functional additional domains may include domains having various purposes that do not directly affect the ability of the ICR repressor to repress immune cell activation including, but not limited to, e.g., structural functions, linker functions, etc.

### Intracellular Inhibitory Domain

An inhibitory domain suitable for use in a synthetic ICR repressor of a subject repressible ICR may be any functional unit of a polypeptide as short as a 3 amino acid linear motif and as long as an entire protein, where size of the stimulatory domain is restricted only in that the domain must be sufficiently large as to retain its function and sufficiently small so as to be compatible with the other components of the repressible ICR. Accordingly, an inhibitory domain may range in size from 3 amino acids in length to 1000 amino acids or more and, in some instances, can have a length of from about 30 amino acids to about 70 amino acids (aa), e.g., an inhibitory domain can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa. In other cases, stimulatory domain can have a length of from about 70 aa to about 100 aa, from about 100 aa to about 200 aa, or greater than 200 aa.

In some instances, “co-inhibitory domains” find use in the synthetic ICR repressor of the present disclosure. Such co-inhibitory domains are generally polypeptides derived from receptors. Co-inhibition generally refers to the secondary inhibition of primary antigen-specific activation mechanisms which prevents co-stimulation. Co-inhibition, e.g., T cell co-inhibition, and the factors involved have been described in Chen & Flies. Nat Rev Immunol (2013) 13(4):227-42 and Thaventhiran et al. J Clin Cell Immunol (2012) S12, the disclosures of which are incorporated herein by reference in their entirety. In some embodiments, co-inhibitory domains homodimerize. A subject co-inhibitory domain can be an intracellular portion of a transmembrane protein (i.e., the co-inhibitory domain can be derived from a transmembrane protein). Non-limiting examples of suitable co-inhibitory polypeptides include, but are not limited to, CTLA-4 and PD-1. In some instances, a co-inhibitory domain, e.g., as used in a synthetic ICR repressor of the instant disclosure may include a co-inhibitory domain listed in Table 1. In some instances, a co-inhibitory domain of a synthetic ICR repressor comprises an amino acid sequence having at least about 75%, at least about 80%, at least about 85%, at least about 90%, at least about 95%, at least about 98%, or 100% amino acid sequence identity to a co-inhibitory domain as described herein.

In some instances, a synthetic ICR repressor may contain an intracellular signaling domain, e.g., a co-inhibitory domain, derived from an intracellular portion of the transmembrane protein PD-1 (also known as CD279, programed cell death 1; etc.). For example, a suitable co-inhibitory domain can comprise an amino acid sequence having at least about 75%, at least about 80%, at least about 85%, at least about 90%, at least about 95%, at least about 98%, or 100% amino acid sequence identity to the following amino acid sequence: ICSRAARGTIGARRTGQPLKEDPSAVPVFSVDYGELDFQWREKTPEPPVPCVPEQTEYA TIVFPSGMGTSSPARRGSADGPRSAQPLRPEDGHCSWPL (SEQ ID NO:18). In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, or from about 95 aa to about 100 aa.

In some instances, a synthetic ICR repressor may contain an intracellular signaling domain, e.g., a co-inhibitory domain, derived from an intracellular portion of the transmembrane protein CTLA4 (also known as CD152, Cytotoxic T-lymphocyte protein 4, Cytotoxic T-lymphocyte-associated antigen 4; etc.). For example, a suitable co-inhibitory domain can comprise an amino acid sequence having at least about 75%, at least about 80%, at least about 85%, at least about 90%, at least about 95%, at least about 98%, or 100% amino acid sequence identity to the following amino acid sequence: SLSKMLKKRSPLTTGVYVKMPPTEPECEKQFQPYFIPIN (SEQ ID NO:6). In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

In some instances, a synthetic ICR repressor may contain an intracellular signaling domain, e.g., a co-inhibitory domain, derived from an intracellular portion of the transmembrane protein HPK1 (also known as MAP4K1, Mitogen-activated protein kinase kinase kinase kinase 1, Hematopoietic progenitor kinase, MAPK/ERK kinase kinase kinase 1, MEK kinase kinase 1, MEKKK 1; etc.). For example, a suitable co-inhibitory domain can comprise an amino acid sequence having at least about 75%, at least about 80%, at least about 85%, at least about 90%, at least about 95%, at least about 98%, or 100% amino acid sequence identity to the following amino acid sequence: YDLLQRLGGGTYGEVFKARDKVSGDLVALKMVKMEPDDDVSTLQKEILILKTCRHANI VAYHGSYLWLQKLWICMEFCGAGSLQDIYQVTGSLSELQISYVCREVLQGLAYLHSQK KIHRDIKGANILINDAGEVRLADFGISAQIGATLARRLSFIGTPYWMAPEVAAVALKGG YNELCDIWSLGITAIELAELQPPLFDVHPLRVLFLMTKSGYQPPRLKEKGKWSAAFHNFI KVTLTKSPKKRPSATKMLSHQLV (SEQ ID NO:137). In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165 aa, from about 165 aa to about 170 aa, from about 170 aa to about 175 aa, from about 175 aa to about 180 aa, from about 180 aa to about 185 aa, from about 185 aa to about 190 aa, from about 190 aa to about 195 aa, from about 195 aa to about 200 aa, from about 200 aa to about 205 aa, from about 205 aa to about 210 aa, from about 210 aa to about 215 aa, from about 215 aa to about 220 aa, from about 220 aa to about 225 aa, from about 225 aa to about 230 aa, from about 230 aa to about 235 aa, from about 235 aa to about 240 aa, from about 240 aa to about 245 aa, from about 245 aa to about 250 aa, from about 250 aa to about 255 aa or from about 255 aa to about 258 aa.

In some instances, a synthetic ICR repressor may contain an intracellular signaling domain, e.g., a co-inhibitory domain, derived from an intracellular portion of the transmembrane protein SHP1 (also known as PTN6, Tyrosine-protein phosphatase non-receptor type 6, Hematopoietic cell protein-tyrosine phosphatase, Protein-tyrosine phosphatase 1C, PTP-1C, SH-PTP1, HCP, PTP1C; etc.). For example, a suitable co-inhibitory domain can comprise an amino acid sequence having at least about 75%, at least about 80%, at least about 85%, at least about 90%, at least about 95%, at least about 98%, or 100% amino acid sequence identity to the following amino acid sequence: FWEEFESLQKQEVKNLHQRLEGQRPENKGKNRYKNILPFDHSRVILQGRDSNIPGSDYI NANYIKNQLLGPDENAKTYIASQGCLEATVNDFWQMAWQENSRVIVMTTREVEKGRN KCVPYWPEVGMQRAYGPYSVTNCGEHDTTEYKLRTLQVSPLDNGDLIREIWHYQYLS WPDHGVPSEPGGVLSFLDQINQRQESLPHAGPIIVHCSAGIGRTGTIIVIDMLMENISTKG LDCDIDIQKTIQMVRAQRSGMVQTEAQYKFIYVAIAQF (SEQ ID NO:138). In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165 aa, from about 165 aa to about 170 aa, from about 170 aa to about 175 aa, from about 175 aa to about 180 aa, from about 180 aa to about 185 aa, from about 185 aa to about 190 aa, from about 190 aa to about 195 aa, from about 195 aa to about 200 aa, from about 200 aa to about 205 aa, from about 205 aa to about 210 aa, from about 210 aa to about 215 aa, from about 215 aa to about 220 aa, from about 220 aa to about 225 aa, from about 225 aa to about 230 aa, from about 230 aa to about 235 aa, from about 235 aa to about 240 aa, from about 240 aa to about 245 aa, from about 245 aa to about 250 aa, from about 250 aa to about 255 aa, from about 255 aa to about 260 aa, from about 260 aa to about 265 aa, from about 265 aa to about 270 aa or from about 270 aa to about 272 aa.

In some instances, a synthetic ICR repressor may contain an intracellular signaling domain, e.g., a co-inhibitory domain, derived from an intracellular portion of the transmembrane protein SHP2 (also known as PTN11, Tyrosine-protein phosphatase non-receptor type 11, Protein-tyrosine phosphatase 1D, PTP-1D, Protein-tyrosine phosphatase 2C, PTP-2C, SH-PTP2, SHP-2, SH-PTP3, PTP2C, SHPTP2; etc.). For example, a suitable co-inhibitory domain can comprise an amino acid sequence having at least about 75%, at least about 80%, at least about 85%, at least about 90%, at least about 95%, at least about 98%, or 100% amino acid sequence identity to the following amino acid sequence: FWEEFETLQQQECKLLYSRKEGQRQENKNKNRYKNILPFDHTRVVLHDGDPNEPVSDY INANIIMPEFETKCNNSKPKKSYIATQGCLQNTVNDFWRMVFQENSRVIVMTTKEVERG KSKCVKYWPDEYALKEYGVMRVRNVKESAAHDYTLRELKLSKVGQALLQGNTERTV WQYHFRTWPDHGVPSDPGGVLDFLEEVHHKQESIMDAGPVVVHCSAGIGRTGTFIVIDI LIDIIREKGVDCDIDVPKTIQMVRSQRSGMVQTEAQYRFIYMA (SEQ ID NO:139). In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165 aa, from about 165 aa to about 170 aa, from about 170 aa to about 175 aa, from about 175 aa to about 180 aa, from about 180 aa to about 185 aa, from about 185 aa to about 190 aa, from about 190 aa to about 195 aa, from about 195 aa to about 200 aa, from about 200 aa to about 205 aa, from about 205 aa to about 210 aa, from about 210 aa to about 215 aa, from about 215 aa to about 220 aa, from about 220 aa to about 225 aa, from about 225 aa to about 230 aa, from about 230 aa to about 235 aa, from about 235 aa to about 240 aa, from about 240 aa to about 245 aa, from about 245 aa to about 250 aa, from about 250 aa to about 255 aa, from about 255 aa to about 260 aa, from about 260 aa to about 265 aa, from about 265 aa to about 270 aa or from about 270 aa to about 275 aa.

In some instances, a synthetic ICR repressor may contain an intracellular signaling domain, e.g., a co-inhibitory domain, derived from an intracellular portion of the transmembrane protein Sts1 (also known as UBS3B, Ubiquitin-associated and SH3 domain-containing protein B, Cbl-interacting protein p70, Suppressor of T-cell receptor signaling 1, STS-1, T-cell ubiquitin ligand 2, TULA-2, Tyrosine-protein phosphatase STS1/TULA2, UBASH3B, KIAA1959; etc.). For example, a suitable co-inhibitory domain can comprise an amino acid sequence having at least about 75%, at least about 80%, at least about 85%, at least about 90%, at least about 95%, at least about 98%, or 100% amino acid sequence identity to the following amino acid sequence: GPQKRCLFVCRHGERMDVVFGKYWLSQCFDAKGRYIRTNLNMPHSLPQRSGGFRDYE KDAPITVFGCMQARLVGEALLESNTIIDHVYCSPSLRCVQTAHNILKGLQQENHLKIRVE PGLFEWTKWVAGSTLPAWIPPSELAAANLSVDTTYRPHIPISKLVVSESYDTYISRSFQV TKEIISECKSKGNNILIVAHASSLEACTCQLQGLSPQNSKDFVQMVRKIPYLGFCSCEELG ETGIWQLTDPPILPLTHGPTGGFNWRETLLQE (SEQ ID NO:140). In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165 aa, from about 165 aa to about 170 aa, from about 170 aa to about 175 aa, from about 175 aa to about 180 aa, from about 180 aa to about 185 aa, from about 185 aa to about 190 aa, from about 190 aa to about 195 aa, from about 195 aa to about 200 aa, from about 200 aa to about 205 aa, from about 205 aa to about 210 aa, from about 210 aa to about 215 aa, from about 215 aa to about 220 aa, from about 220 aa to about 225 aa, from about 225 aa to about 230 aa, from about 230 aa to about 235 aa, from about 235 aa to about 240 aa, from about 240 aa to about 245 aa, from about 245 aa to about 250 aa, from about 250 aa to about 255 aa, from about 255 aa to about 260 aa, from about 260 aa to about 265 aa or from about 265 aa to about 270 aa.

In some instances, a synthetic ICR repressor may contain an intracellular signaling domain, e.g., a co-inhibitory domain, derived from an intracellular portion of the transmembrane protein Csk (also known as Tyrosine-protein kinase CSK, C-Src kinase, Protein-tyrosine kinase CYL; etc.). For example, a suitable co-inhibitory domain can comprise an amino acid sequence having at least about 75%, at least about 80%, at least about 85%, at least about 90%, at least about 95%, at least about 98%, or 100% amino acid sequence identity to the following amino acid sequence: LKLLQTIGKGEFGDVMLGDYRGNKVAVKCIKNDATAQAFLAEASVMTQLRHSNLVQL LGVIVEEKGGLYIVTEYMAKGSLVDYLRSRGRSVLGGDCLLKFSLDVCEAMEYLEGNN FVHRDLAARNVLVSEDNVAKVSDFGLTKEASSTQDTGKLPVKWTAPEALREKKFSTKS DVWSFGILLWEIYSFGRVPYPRIPLKDVVPRVEKGYKMDAPDGCPPAVYEVMKNCWH LDAAMRPSFLQLREQLEHIKTHELH (SEQ ID NO:141). In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165 aa, from about 165 aa to about 170 aa, from about 170 aa to about 175 aa, from about 175 aa to about 180 aa, from about 180 aa to about 185 aa, from about 185 aa to about 190 aa, from about 190 aa to about 195 aa, from about 195 aa to about 200 aa, from about 200 aa to about 205 aa, from about 205 aa to about 210 aa, from about 210 aa to about 215 aa, from about 215 aa to about 220 aa, from about 220 aa to about 225 aa, from about 225 aa to about 230 aa, from about 230 aa to about 235 aa, from about 235 aa to about 240 aa, from about 240 aa to about 245 aa, from about 245 aa to about 250 aa or from about 250 aa to about 255 aa.

In some instances, a synthetic ICR repressor may contain an intracellular signaling domain, e.g., a co-inhibitory domain, derived from an intracellular portion of a transmembrane protein listed in Table 1. For example, a suitable co-inhibitory domain can comprise an amino acid sequence having at least about 75%, at least about 80%, at least about 85%, at least about 90%, at least about 95%, at least about 98%, or 100% amino acid sequence identity to an amino acid sequence listed in Table 1. In some of these embodiments, the co-inhibitory domain has a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa, from about 70 aa to about 75 aa, from about 75 aa to about 80 aa, from about 80 aa to about 85 aa, from about 85 aa to about 90 aa, from about 90 aa to about 95 aa, from about 95 aa to about 100 aa, from about 100 aa to about 105 aa, from about 105 aa to about 110 aa, from about 110 aa to about 115 aa, from about 115 aa to about 120 aa, from about 120 aa to about 125 aa, from about 125 aa to about 130 aa, from about 130 aa to about 135 aa, from about 135 aa to about 140 aa, from about 140 aa to about 145 aa, from about 145 aa to about 150 aa, from about 150 aa to about 155 aa, from about 155 aa to about 160 aa, from about 160 aa to about 165, aa from about 165 aa to about 170 aa, from about 170 aa to about 175 aa, from about 175 aa to about 180 aa, from about 180 aa to about 185 aa, or from about 185 aa to about 190 aa.

### Transmembrane Domain

Any transmembrane (TM) domain that provides for insertion of a polypeptide into the cell membrane of a eukaryotic (e.g., mammalian) cell is suitable for use. As one non-limiting example, the TM sequence IYIWAPLAGTCGVLLLSLVITLYC (SEQ ID NO:98) can be used. Additional non-limiting examples of suitable TM sequences include:

a) CD8 beta derived:
(SEQ ID NO: 99)
LGLLVAGVLVLLVSLGVAIHLCC;
b) CD4 derived:
(SEQ ID NO: 100)
ALIVLGGVAGLLLFIGLGIFFCVRC;
c) CD3 zeta derived:
(SEQ ID NO: 101)
LCYLLDGILFIYGVILTALFLRV;
d) CD28 derived:
(SEQ ID NO: 102)
WVLVVVGGVLACYSLLVTVAFIIFWV;
e) CD134 (OX40) derived:
(SEQ ID NO: 103)
VAAILGLGLVLGLLGPLAILLALYLL;
and
f) CD7 derived:
(SEQ ID NO: 104)
ALPAALAVISFLLGLGLGVACVLA.

### Linkers

In some cases, a subject synthetic ICR repressor includes a linker between any two adjacent domains. For example, a linker can be disposed between the transmembrane domain, where present, and the first intracellular functional domain, e.g., a co-inhibitory domain, of the synthetic ICR repressor. As another example, a linker can be disposed between a first intracellular functional domain and the member of the dimerization domain of the synthetic ICR repressor. As another example, a linker can be disposed the transmembrane domain, where present, and the member of the dimerization domain of the synthetic ICR repressor. As another example, a linker can be disposed between the member of the dimerization domain and a second intracellular functional domain, e.g., an immune cell negative regulatory domain. As another example, a linker can be disposed between any domain of the synthetic ICR repressor and any additional domain including e.g., a domain not involved in the primary immune repression functions of the synthetic ICR repressor including but not limited to e.g., a reporter domain, a tag domain, etc.

Linkers may be utilized in a suitable configuration in the synthetic ICR repressor provided they do not abolish the primary activities of the synthetic ICR repressor including, e.g., the ability of the synthetic ICR repressor to repress an activated ICR, the ability of the dimerization domain of the synthetic ICR repressor to bind the dimerization domain of the repressible ICR.

Any suitable linker, including two or more linkers (e.g., where the two or more linkers are the same or different and including where the multiple linkers are three or more, four or more, five or more, six or more, etc. and including where all the linkers are different and where the multiple linkers include an mix of some linkers utilized in more than one location and some linkers utilized specifically in only one location and the like) may be utilized in the subject synthetic ICR repressors including e.g., those linkers described herein for acceptable use in a CAR.

### Dimerizer/Dimerization Pair

Dimerizers (“dimerizing agents) that can provide for dimerization of a first member of a dimerizer-binding pair and a second member of a dimerizer-binding pair include, e.g. (where the dimerizer is in parentheses following the dimerizer-binding pair: FKBP and FKBP (rapamycin); FKBP and CnA (rapamycin); FKBP and cyclophilin (rapamycin); FKBP and FRG (rapamycin); GyrB and GyrB (coumermycin); DHFR and DHFR (methotrexate); DmrB and DmrB (AP20187); PYL and ABI (abscisic acid); Cry2 and CIB1 (blue light); and GAI and GID1 (gibberellin), and those described in PCT Pub. No. WO 2015/090229, the disclosure of which is incorporated herein by reference in its entirety.

As noted above, rapamycin can serve as a dimerizer. Alternatively, a rapamycin derivative or analog can be used. See, e.g., WO96/41865; WO 99/36553; WO 01/14387; and Ye et al (1999) Science 283:88-91. For example, analogs, homologs, derivatives and other compounds related structurally to rapamycin (“rapalogs”) include, among others, variants of rapamycin having one or more of the following modifications relative to rapamycin: demethylation, elimination or replacement of the methoxy at C7, C42 and/or C29; elimination, derivatization or replacement of the hydroxy at C13, C43 and/or C28; reduction, elimination or derivatization of the ketone at C14, C24 and/or C30; replacement of the 6-membered pipecolate ring with a 5-membered prolyl ring; and alternative substitution on the cyclohexyl ring or replacement of the cyclohexyl ring with a substituted cyclopentyl ring. Additional information is presented in, e.g., U.S. Pat. Nos. 5,525,610; 5,310,903 5,362,718; and 5,527,907. Selective epimerization of the C-28 hydroxyl group has been described; see, e.g., WO 01/14387. Additional synthetic dimerizing agents suitable for use as an alternative to rapamycin include those described in U.S. Patent Publication No. 2012/0130076.

Rapamycin has the structure:

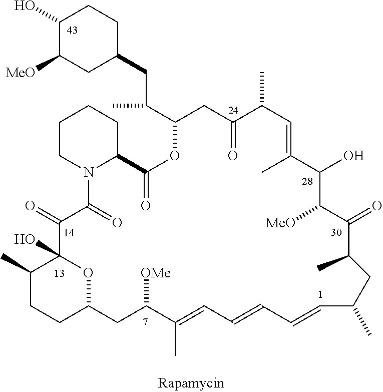

Suitable rapalogs include, e.g.,

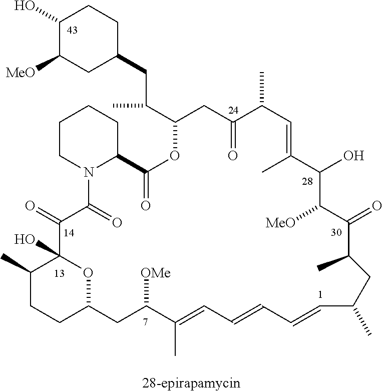

Also suitable as a rapalog is a compound of the formula:

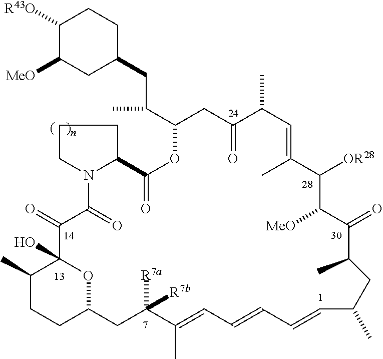

where n is 1 or 2; R²⁸ and R⁴³ are independently H, or a substituted or unsubstituted aliphatic or acyl moiety; one of R^{7a} and R^{7b} is H and the other is halo, R^{A}, OR^{A}, SR^{A}, —OC(O)R^{A}, —OC(O)NR^{A}R^{B}, —NR^{A}R^{B}, —NR^{B}C(OR)R^{A}, NR^{B}C(O)OR^{A}, —NR^{B}SO₂R^{A}, or NR^{B}SO₂NR^{A}R^{B}; or R^{7a} and R^{7b}, taken together, are H in the tetraene moiety:

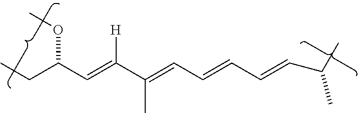

where R^{A} is H or a substituted or unsubstituted aliphatic, heteroaliphatic, aryl, or heteroaryl moiety and where R^{B} and R^{B′} are independently H, OH, or a substituted or unsubstituted aliphatic, heteroaliphatic, aryl, or heteroaryl moiety.

As noted above, coumermycin can serve as a dimerizing agent. Alternatively, a coumermycin analog can be used. See, e.g., Farrar et al. (1996) Nature 383:178-181; and U.S. Pat. No. 6,916,846.

As noted above, in some cases, the dimerizing agent is methotrexate, e.g., a non-cytotoxic, homo-bifunctional methotrexate dimer. See, e.g., U.S. Pat. No. 8,236,925.

In some instances, the members of a dimerization pair may be or may include a ligand-binding domain (LBD) of a nuclear hormone receptor, e.g., where the the first member of the dimerization pair comprises a LBD of a nuclear hormone receptor, and the second member of the dimerization pair comprises a co-regulator of the nuclear hormone receptor, or wherein the first member of the dimerization pair is a co-regulator of a nuclear hormone receptor, and the second member of the dimerization pair comprises an LBD of the nuclear hormone receptor; and wherein the two molecules that include the first and second members of the dimerization pair are dimerized in the presence of a dimerization agent that induces binding of the LBD to the co-regulator.

A ligand-binding domain of a nuclear hormone receptor can be from any of a variety of nuclear hormone receptors, including, but not limited to, ERα, ERβ, PR, AR, GR, MR, RARα, RARβ, RARγ, TRα, TRβ, VDR, EcR, RXRα, RXRβ, RXRγ, PPARα, PPARβ, PPARγ, LXRα, LXRβ, FXR, PXR, SXR, CAR, SF-1, LRH-1, DAX-1, SHP, TLX, PNR, NGF1-Bα, NGF1-Bβ, NGF1-Bγ, RORα, RORβ, RORγ, ERRα, ERRβ, ERRγ, GCNF, TR2/4, HNF-4, COUP-TFα, COUP-TFβ and COUP-TFγ. Abbreviations for nuclear hormone receptors are as follows. ER: Estrogen Receptor; PR: Progesterone Receptor; AR: Androgen Receptor; GR: Glucocorticoid Receptor; MR: Mineralocorticoid Receptor; RAR: Retinoic Acid Receptor; TRα, β: Thyroid Receptor; VDR: Vitamin D3 Receptor; EcR: Ecdysone Receptor; RXR: Retinoic Acid X Receptor; PPAR: Peroxisome Proliferator Activated Receptor; LXR: Liver X Receptor; FXR: Farnesoid X Receptor; PXR/SXR: Pregnane X Receptor/Steroid and Xenobiotic Receptor; CAR: Constitutive Adrostrane Receptor; SF-1: Steroidogenic Factor 1; DAX-1: Dosage sensitive sex reversal-adrenal hypoplasia congenital critical region on the X chromosome, gene 1; LRH-1: Liver Receptor Homolog 1; SHP: Small Heterodimer Partner; TLX: Tail-less Gene; PNR: Photoreceptor-Specific Nuclear Receptor; NGF1-B: Nerve Growth Factor; ROR: RAR related orphan receptor; ERR: Estrogen Related Receptor; GCNF: Germ Cell Nuclear Factor; TR2/4: Testicular Receptor; HNF-4: Hepatocyte Nuclear Factor; COUP-TF: Chicken Ovalbumin Upstream Promoter, Transcription Factor.

In some instances, the LBD of the nuclear hormone binding member of the dimerization pair is an LBD of a nuclear hormone receptor selected from an estrogen receptor, an ecdysone receptor, a PPARγ receptor, a glucocorticoid receptor, an androgen receptor, a thyroid hormone receptor, a mineralocorticoid receptor, a progesterone receptor, a vitamin D receptor, a PPARβ receptor, a PPARα receptor, a pregnane X receptor, a liver X receptor, a farnesoid X receptor, a retinoid X receptor, a RAR-related orphan receptor, and a retinoic acid receptor. In some cases, the co-regulator of the nuclear hormone receptor is selected from SRC1, GRIP1, AIB1, PGC1a, PGC1b, PRC, TRAP220, ASC2, CBP, P300, CIA, ARA70, TIF1, NSD1, SMAP, Tip60, ERAP140, Nix1, LCoR, N-CoR, SMRT, RIP140, and PRIC285.

In some cases, an LBD suitable for inclusion as a member of a dimerization pair of a polypeptide of the present disclosure is an LBD of estrogen receptor-alpha (ERα). For example, in some cases, the LBD comprises an amino acid sequence having at least 75%, at least 80%, at least 85%, at least 90%, at least 95%, at least 98%, at least 99%, or 100%, amino acid sequence identity to the LBD of an ERα having the amino acid sequence depicted in FIG. 32.

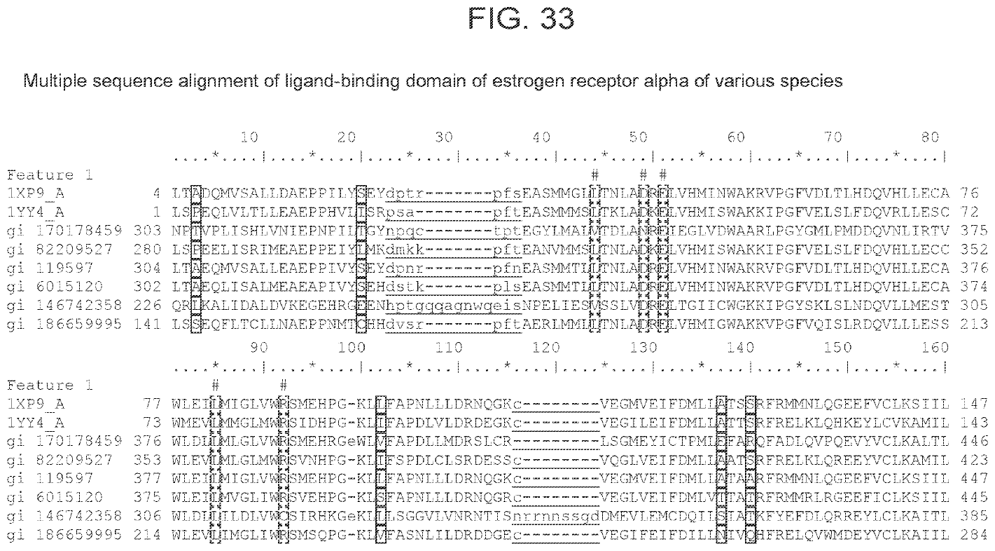

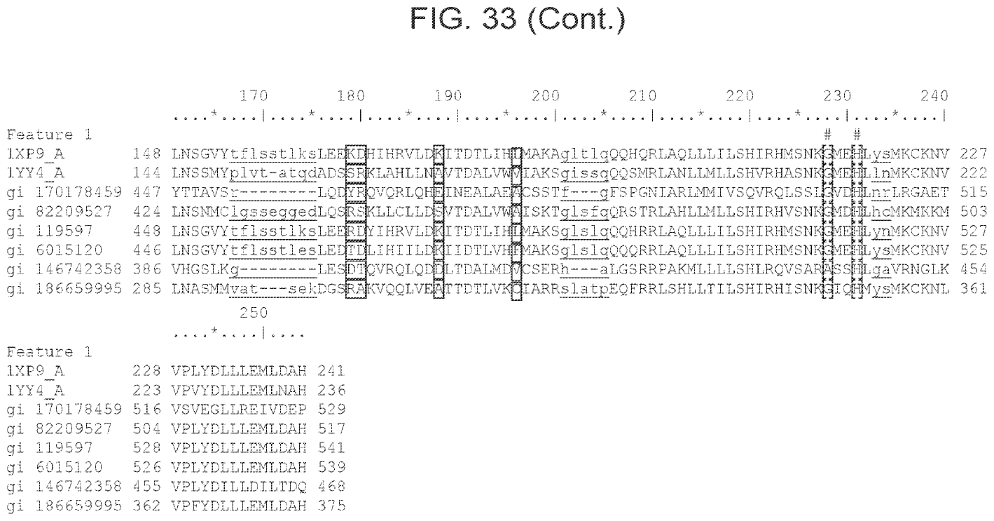

As one non-limiting example, the LBD of an ERα can comprise an amino acid sequence having at least 75%, at least 80%, at least 85%, at least 90%, at least 95%, at least 98%, at least 99%, or 100%, amino acid sequence identity to an amino acid sequence depicted in FIG. 33; and has a length of from about 200 amino acids to 240 amino acids (e.g., has a length of from 200 amino acids to 225 amino acids, from 225 amino acids to 230 amino acids, from 230 amino acids to 235 amino acids, or from 235 amino acids to 240 amino acids).

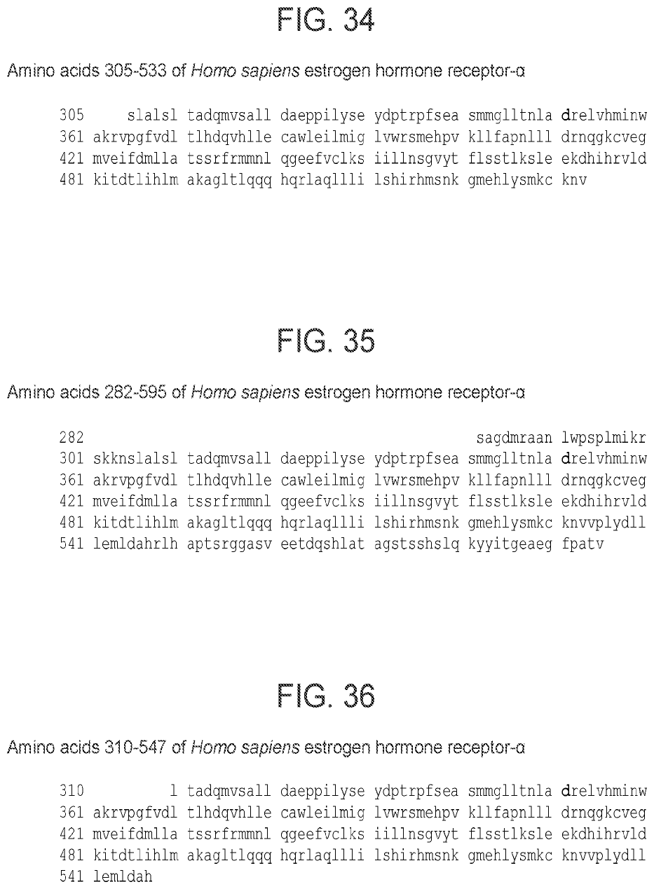

As one non-limiting example, the LBD of an ERα can comprise an amino acid sequence having at least 75%, at least 80%, at least 85%, at least 90%, at least 95%, at least 98%, at least 99%, or 100%, amino acid sequence identity to the amino acid sequence depicted in FIG. 34; and has a length of from about 180 amino acids to 229 amino acids (e.g., has a length of from 180 amino acids to 200 amino acids, or from 200 amino acids to 229 amino acids; e.g., has a length of 229 amino acids).

As one non-limiting example, the LBD of an ERα can comprise an amino acid sequence having at least 75%, at least 80%, at least 85%, at least 90%, at least 95%, at least 98%, at least 99%, or 100%, amino acid sequence identity to the amino acid sequence depicted in FIG. 35; and has a length of from about 250 amino acids to 314 amino acids (e.g., has a length of from 250 amino acids to 275 amino acids, from 275 amino acids to 300 amino acids, or from 300 amino acids to 314 amino acids; e.g., has a length of 314 amino acids).

As one non-limiting example, the LBD of an ERα can comprise an amino acid sequence having at least 75%, at least 80%, at least 85%, at least 90%, at least 95%, at least 98%, at least 99%, or 100%, amino acid sequence identity to the amino acid sequence depicted in FIG. 36; and has a length of from about 190 amino acids to 238 amino acids (e.g., has a length of from 190 amino acids to 220 amino acids, or from 220 amino acids to 238 amino acids; e.g., has a length of 238 amino acids).

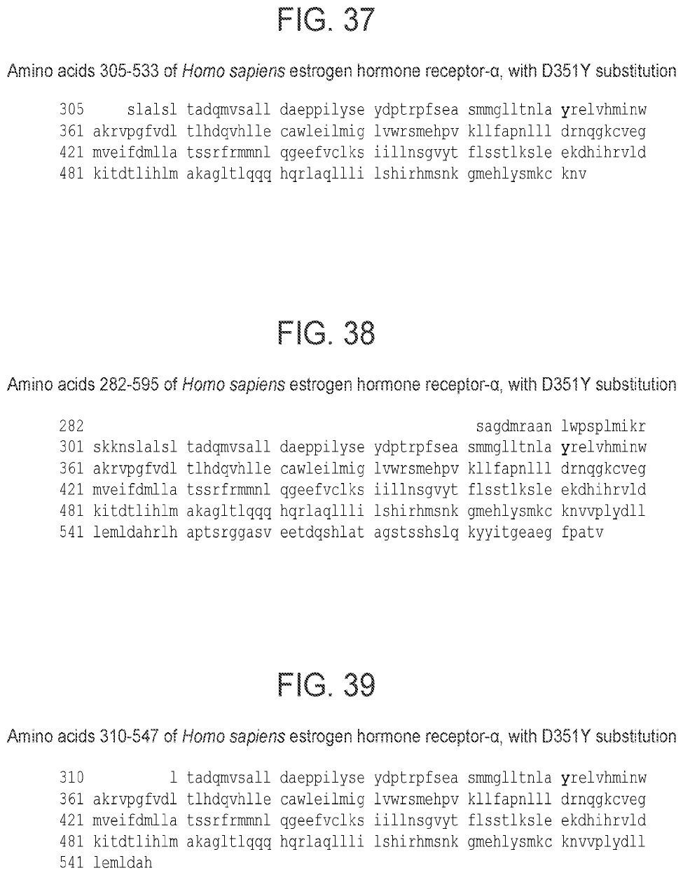

As one non-limiting example, the LBD of an ERα can comprise an amino acid sequence having at least 75%, at least 80%, at least 85%, at least 90%, at least 95%, at least 98%, at least 99%, or 100%, amino acid sequence identity to the amino acid sequence depicted in FIG. 37, and has a D351Y substitution (where the amino acid numbering is based on the amino acid sequence depicted in FIG. 32); and has a length of from about 180 amino acids to 229 amino acids (e.g., has a length of from 180 amino acids to 200 amino acids, or from 200 amino acids to 229 amino acids; e.g., has a length of 229 amino acids).

As one non-limiting example, the LBD of an ERα can comprise an amino acid sequence having at least 75%, at least 80%, at least 85%, at least 90%, at least 95%, at least 98%, at least 99%, or 100%, amino acid sequence identity to the amino acid sequence depicted in FIG. 38, and has a D351Y substitution (where the amino acid numbering is based on the amino acid sequence depicted in FIG. 32); and has a length of from about 250 amino acids to 314 amino acids (e.g., has a length of from 250 amino acids to 275 amino acids, from 275 amino acids to 300 amino acids, or from 300 amino acids to 314 amino acids; e.g., has a length of 314 amino acids).

As one non-limiting example, the LBD of an ERα can comprise an amino acid sequence having at least 75%, at least 80%, at least 85%, at least 90%, at least 95%, at least 98%, at least 99%, or 100%, amino acid sequence identity to the amino acid sequence depicted in FIG. 39, and has a D351Y substitution (where the amino acid numbering is based on the amino acid sequence depicted in FIG. 32); and has a length of from about 190 amino acids to 238 amino acids (e.g., has a length of from 190 amino acids to 220 amino acids, or from 220 amino acids to 238 amino acids; e.g., has a length of 238 amino acids).

In some cases, where the first member of a dimerization pair of a polypeptide of the present disclosure is an LBD of an ERα, the second member of the dimerization pair is a co-regulator peptide comprising the amino acid sequence DAFQLRQLILRGLQDD (SEQ ID NO:151), where the co-regulator peptide has a length of from about 16 amino acids to about 50 amino acids (e.g., the co-regulator peptide has a length of from 16 amino acids to 20 amino acids, from 20 amino acids to 25 amino acids, from 25 amino acids to 30 amino acids, from 30 amino acids to 35 amino acids, from 35 amino acids to 40 amino acids, from 40 amino acids to 45 amino acids, or from 45 amino acids to 50 amino acids). In some cases, where the second member of a dimerization pair of a polypeptide of the present disclosure is an LBD of an ERα, the first member of the dimerization pair is a co-regulator peptide comprising the amino acid sequence DAFQLRQLILRGLQDD (SEQ ID NO:152), where the co-regulator peptide has a length of from about from about 16 amino acids to about 50 amino acids (e.g., the co-regulator peptide has a length of from 16 amino acids to 20 amino acids, from 20 amino acids to 25 amino acids, from 25 amino acids to 30 amino acids, from 30 amino acids to 35 amino acids, from 35 amino acids to 40 amino acids, from 40 amino acids to 45 amino acids, or from 45 amino acids to 50 amino acids).

In some cases, where the first member of a dimerization pair of a polypeptide of the present disclosure is an LBD of an ERα, the second member of the dimerization pair is a co-regulator peptide comprising the amino acid sequence SPGSREWFKDMLS (SEQ ID NO:153), where the co-regulator peptide has a length of from about 13 amino acids to about 50 amino acids (e.g., the co-regulator peptide has a length of from 13 amino acids to 15 amino acids, from 15 amino acids to 20 amino acids, from 20 amino acids to 25 amino acids, from 25 amino acids to 30 amino acids, from 30 amino acids to 35 amino acids, from 35 amino acids to 40 amino acids, from 40 amino acids to 45 amino acids, or from 45 amino acids to 50 amino acids). In some cases, where the second member of a dimerization pair of a polypeptide of the present disclosure is an LBD of an ERα, the first member of the dimerization pair is a co-regulator peptide comprising the amino acid sequence SPGSREWFKDMLS (SEQ ID NO:154), where the co-regulator peptide has a length of from about from about 13 amino acids to about 50 amino acids (e.g., the co-regulator peptide has a length of from 13 amino acids to 15 amino acids, from 15 amino acids to 20 amino acids, from 20 amino acids to 25 amino acids, from 25 amino acids to 30 amino acids, from 30 amino acids to 35 amino acids, from 35 amino acids to 40 amino acids, from 40 amino acids to 45 amino acids, or from 45 amino acids to 50 amino acids).

In some cases, a polypeptide chain of the present disclosure comprises a single LBD of a nuclear hormone receptor. In some cases, a polypeptide chain of a heterodimeric polypeptide of the present disclosure comprises multiple (two or more) LBD of a nuclear hormone receptor. In some cases, a polypeptide chain of a polypeptide of the present disclosure comprises two LBD of a nuclear hormone receptor. In some cases, a polypeptide chain of a polypeptide of the present disclosure comprises three LBD of a nuclear hormone receptor. Where a polypeptide chain of a polypeptide of the present disclosure comprises multiple (two or more) LBD of a nuclear hormone receptor, in some cases the multiple LBD comprise identical amino acid sequences. In some cases, the two or more LBD are in tandem, either directly or separated by a linker.

Suitable co-regulator polypeptides include full-length naturally-occurring nuclear hormone co-regulator polypeptides. Suitable co-regulator polypeptides include fragments of naturally-occurring nuclear hormone co-regulator polypeptides. Suitable co-regulator polypeptides include synthetic or recombinant nuclear hormone co-regulator polypeptides.

The co-regulator nuclear hormone receptor polypeptides useful in the polypeptide chains of the present disclosure will vary and may include but are not limited to the following, including but not limited to variants of the following having 80% or more sequence identity to a sequence of the following (including but not limited to e.g., 85% sequence identity of more, 90% sequence identity or more, 99% sequence identity or more, etc.):

SRC1:
(SEQ ID NO: 155)
CPSSHSSLTERHKILHRLLQEGSPS;
SRC1-2:
(SEQ ID NO: 156)
SLTARHKILHRLLQEGSPSDI;
SRC3-1:
(SEQ ID NO: 157)
ESKGHKKLLQLLTCSSDDR;
SRC3:
(SEQ ID NO: 158)
PKKENNALLRYLLDRDDPSDV;
PGC-1:
(SEQ ID NO: 159)
AEEPSLLKKLLLAPANT;
PGC1a:
(SEQ ID NO: 160)
QEAEEPSLLKKLLLAPANTQL;
TRAP220-1:
(SEQ ID NO: 161)
SKVSQNPILTSLLQITGNGGS;
NCoR (2051-2075):
(SEQ ID NO: 162)
GHSFADPASNLGLEDIIRKALMGSF;
NR0B1:
(SEQ ID NO: 163)
PRQGSILYSMLTSAKQT;
NRIP1:
(SEQ ID NO: 164)
AANNSLLLHLLKSQTIP;
TIF2:
(SEQ ID NO: 165)
PKKKENALLRYLLDKDDTKDI;
CoRNR Box:
(SEQ ID NO: 166)
DAFQLRQLILRGLQDD;
abV:
(SEQ ID NO: 167)
SPGSREWFKDMLS;
TRAP220-2:
(SEQ ID NO: 168)
GNTKNHPMLMNLLKDNPAQDF;
EA2:
(SEQ ID NO: 169)
SSKGVLWRMLAEPVSR;
TA1:
(SEQ ID NO: 170)
SRTLQLDWGTLYWSR;
EAB1:
(SEQ ID NO: 171)
SSNHQSSRLIELLSR;
SRC2:
(SEQ ID NO: 172)
LKEKHKILHRLLQDSSSPV;
SRC1-3:
(SEQ ID NO: 173)
QAQQKSLLQQLLTE;
SRC1-1:
(SEQ ID NO: 174)
KYSQTSHK LVQLL TTTAEQQL;
SRC1-2:
(SEQ ID NO: 175)
SLTARHKI LHRLL QEGSPSDI;
SRC1-3:
(SEQ ID NO: 176)
KESKDHQL LRYLL DKDEKDLR;
SRC1-4a:
(SEQ ID NO: 177)
PQAQQKSL LQQLL TE;
SRC1-4b:
(SEQ ID NO: 178)
PQAQQKSL RQQLL TE;
GRIP1-1:
(SEQ ID NO: 179)
HDSKGQTK LLQLL TTKSDQME;
GRIP1-2:
(SEQ ID NO: 180)
SLKEKHKI LHRLL QDSSSPVD;
GRIP1-3:
(SEQ ID NO: 181)
PKKKENAL LRYLL DKDDTKDI;
AIB1-1:
(SEQ ID NO: 182)
LESKGHKK LLQLL TCSSDDRG;
AIB1-2:
(SEQ ID NO: 183)
LLQEKHRI LHKLL QNGNSPAE;
AIB1-3:
(SEQ ID NO: 184)
KKKENNAL LRYLL DRDDPSDA;
PGC1a:
(SEQ ID NO: 185)
QEAEEPSL LKKLL LAPANTQL;
PGC1b:
(SEQ ID NO: 186)
PEVDELSL LQKLL LATSYPTS;
PRC:
(SEQ ID NO: 187)
VSPREGSS LHKLL TLSRTPPE;
TRAP220-1:
(SEQ ID NO: 188)
SKVSQNPI LTSLL QITGNGGS;
TRAP220-2:
(SEQ ID NO: 189)
GNTKNHPM LMNLL KDNPAQDF;
ASC2-1:
(SEQ ID NO: 190)
DVTLTSPL LVNLL QSDISAGH;
ASC2-2:
(SEQ ID NO: 191)
AMREAPTS LSQLL DNSGAPNV;
CBP-1:
(SEQ ID NO: 192)
DAASKHKQ LSELL RGGSGSSI;
CBP-2:
(SEQ ID NO: 193)
KRKLIQQQ LVLLL HAHKCQRR;
P300:
(SEQ ID NO: 194)
DAASKHKQ LSELL RSGSSPNL;
CIA:
(SEQ ID NO: 195)
GHPPAIQS LINLL ADNRYLTA;
ARA70-1:
(SEQ ID NO: 196)
TLQQQAQQ LYSLL GQFNCLTH;
ARA70-2:
(SEQ ID NO: 197)
GSRETSEK FKLLF QSYNVNDW;
TIF1:
(SEQ ID NO: 198)
NANYPRSI LTSLL LNSSQSST;
NSD1:
(SEQ ID NO: 199)
IPIEPDYK FSTLL MMLKDMHD;
SMAP:
(SEQ ID NO: 200)
ATPPPSPL LSELL KKGSLLPT;
Tip60:
(SEQ ID NO: 201)
VDGHERAM LKRLL RIDSKCLH;
ERAP140:
(SEQ ID NO: 202)
HEDLDKVK LIEYY LTKNKEGP;
Nix1:
(SEQ ID NO: 203)
ESPEFCLG LQTLL SLKCCIDL;
LCoR:
(SEQ ID NO: 204)
AATTQNPV LSKLL MADQDSPL;
CoRNR1 (N-CoR):
(SEQ ID NO: 205)
MGQVPRTHRLITLADH ICQII TQDFARNQV;
CoRNR2 (N-CoR):
(SEQ ID NO: 206)
NLG LEDII RKALMG;
CoRNR1 (SMRT):
(SEQ ID NO: 207)
APGVKGHQRVVTLAQH ISEVI TQDTYRHHPQQLSAPLPAP;
CoRNR2 (SMRT):
(SEQ ID NO: 208)
NMG LEAII RKALMG;
RIP140-C:
(SEQ ID NO: 209)
RLTKTNPI LYYML QKGGNSVA;
RIP140-1:
(SEQ ID NO: 210)
QDSIVLTY LEGLL MHQAAGGS;
RIP140-2:
(SEQ ID NO: 211)
KGKQDSTL LASLL QSFSSRLQ;
RIP140-3:
(SEQ ID NO: 212)
CYGVASSH LKTLL KKSKVKDQ;
RIP140-4:
(SEQ ID NO: 213)
KPSVACSQ LALLL SSEAHLQQ;
RIP140-5:
(SEQ ID NO: 214)
KQAANNSL LLHLL KSQTIPKP;
RIP140-6:
(SEQ ID NO: 215)
NSHQKVTL LQLLL GHKNEENV;
RIP140-7:
(SEQ ID NO: 216)
NLLERRTV LQLLL GNPTKGRV;
RIP140-8:
(SEQ ID NO: 217)
FSFSKNGL LSRLL RQNQDSYL;
RIP140-9:
(SEQ ID NO: 218)
RESKSFNV LKQLL LSENCVRD;
PRIC285-1:
(SEQ ID NO: 219)
ELNADDAI LRELL DESQKVMV;
PRIC285-2:
(SEQ ID NO: 220)
YENLPPAA LRKLL RAEPERYR;
PRIC285-3:
(SEQ ID NO: 221)
MAFAGDEV LVQLL SGDKAPEG;
PRIC285-4:
(SEQ ID NO: 222)
SCCYLCIR LEGLL APTASPRP;
and
PRIC285-5:
(SEQ ID NO: 223)
PSNKSVDV LAGLL LRRMELKP.

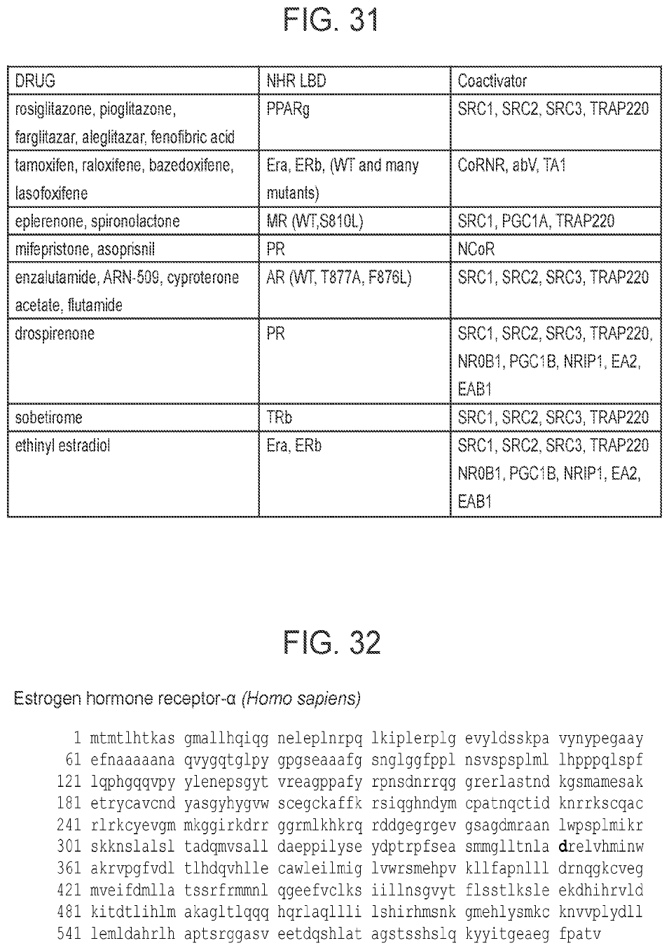

In some cases, a given LBD can be paired with two or more different co-regulator polypeptides. For example, as depicted in FIG. 31, PPARγ can be paired with SRC1, SRC2, SRC3, or TRAP220. As another example, ERα can be paired with CoRNR, αβV, or TA1. As another example, ERβ can be paired with CoRNR, αβV, or TA1. As another example, AR can be paired with SRC1, SRC2, SRC3, or TRAP220. As another example, PR can be paired with SRC1, SRC2, SRC3, TRAP220, NR0B1, PGC1B, NRIP1, EA2, or EAB1. As another example, TRβ can be paired with SRC1, SRC2, SRC3, or TRAP220.

In some cases, a polypeptide of the present disclosure comprises a polypeptide chain comprising multiple (two or more) co-regulator peptides. Where a polypeptide of the present disclosure comprises a polypeptide chain comprising multiple (two or more) co-regulator peptides, the multiple co-regulator peptides can be in tandem, directly or separated by a linker. In some cases, the two or more co-regulator peptides present in the polypeptide chain are identical in amino acid sequence to one another. In some cases, where a polypeptide of the present disclosure comprises a polypeptide chain comprising multiple (two or more) co-regulator peptides, the polypeptide chain comprises two co-regulator peptides. In some cases, where a polypeptide of the present disclosure comprises a polypeptide chain comprising multiple (two or more) co-regulator peptides, the polypeptide chain comprises three co-regulator peptides. In such cases, the second polypeptide chain can comprise multiple (two or more) LBD of a nuclear hormone receptor. For example, where the second polypeptide chain comprises two LBD of a nuclear hormone receptor, the two LBD can be identical in amino acid sequence to one another.

Suitable LBD dimerization agents (also referred to as LBD dimerizing agents; LBD dimerizers) bind the LBD of a nuclear hormone receptor in a first polypeptide and bind the co-regulator peptide in a second polypeptide. Binding of to dimerization agent to the LBD and the co-regulator peptide functions to dimerize the first and second polypeptides the present disclosure (e.g., a first synthetic stimulatory ICR polypeptide and a second synthetic ICR repressor polypeptide).

Examples of dimerization agents include corticosterone (11beta,21-dihydroxy-4-pregnene-3,20-dione); deoxycorticosterone (21-hydroxy-4-pregnene-3,20-dione); cortisol (11beta,17,21-trihydroxy-4-pregnene-3,20-dione); 11-deoxycortisol (17,21-dihydroxy-4-pregnene-3,20-dione); cortisone (17,21-dihydroxy-4-pregnene-3,11,20-trione); 18-hydroxycorticosterone (11beta,18,21-trihydroxy-4-pregnene-3,20-dione); 1.alpha.-hydroxycorticosterone (1alpha,11beta,21-trihydroxy-4-pregnene-3,20-dione); aldosterone 18,11-hemiacetal of 11beta,21-dihydroxy-3,20-dioxo-4-pregnen-18-a1, androstenedione (4-androstene-3,17-dione); 4-hydroxy-androstenedione; 11β-hydroxyandrostenedione (11 beta-4-androstene-3,17-dione); androstanediol (3-beta,17-beta-Androstanediol); androsterone (3alpha-hydroxy-5alpha-androstan-17-one); epiandrosterone (3beta-hydroxy-5alpha-androstan-17-one); adrenosterone (4-androstene-3,11,17-trione); dehydroepiandrosterone (3beta-hydroxy-5-androsten-17-one); dehydroepiandrosterone sulphate (3beta-sulfoxy-5-androsten-17-one); testosterone (17beta-hydroxy-4-androsten-3-one); epitestosterone (17alpha-hydroxy-4-androsten-3-one); 5α-dihydrotestosterone (17beta-hydroxy-5alpha-androstan-3-one 5-dihydrotestosterone; 5-beta-dihydroxy testosterone (17beta-hydroxy-5beta-androstan-3-one); 11β-hydroxytestosterone (11beta,17beta-dihydroxy-4-androsten-3-one); 11-ketotestosterone (17beta-hydroxy-4-androsten-3,17-dione), estrone (3-hydroxy-1,3,5(10)-estratrien-17-one); estradiol (1,3,5(10)-estratriene-3,17beta-diol); estriol 1,3,5(10)-estratriene-3,16alpha,17beta-triol; pregnenolone (3-beta-hydroxy-5-pregnen-20-one); 17-hydroxypregnenolone (3-beta,17-dihydroxy-5-pregnen-20-one); progesterone (4-pregnene-3,20-dione); 17-hydroxyprogesterone (17-hydroxy-4-pregnene-3,20-dione); progesterone (pregn-4-ene-3,20-dione); T3 and T4.

For example, where a polypeptide of the present disclosure comprises an LBD of estrogen receptor-alpha (ERα) and a corresponding co-regulator peptide, a suitable dimerization agent includes, but is not limited to, tamoxifen and analogs thereof, 4-OH-tamoxifen, raloxifene, lasofoxifene, bazedoxifene, falsodex, clomifene, femarelle, ormeloxifene, toremifiene, ospemifene, and ethinyl estradiol.

Additional dimerizer pairs and associated dimerizers that may find use in polypeptides of the present disclosure include but are not limited to e.g., those described in U.S. Provisional Patent Application Ser. No. 62/276,725 and PCT International Pub. No. WO 2015/090229 A1; the disclosures of which are incorporated herein by reference in their entireties.

### Additional Sequences

The heteromeric, conditionally repressible synthetic ICR of the instant disclosure may further include one or more additional polypeptide domains, where such domains include, but are not limited to, a signal sequence; an epitope tag; an affinity domain; and a polypeptide that produces a detectable signal.

### Signal Sequences

Signal sequences that are suitable for use in a subject repressible synthetic ICR, e.g., in the stimulatory ICR or the ICR repressor, include any eukaryotic signal sequence, including a naturally-occurring signal sequence, a synthetic (e.g., man-made) signal sequence, etc.

### Epitope Tag

Suitable epitope tags include, but are not limited to, hemagglutinin (HA; e.g., YPYDVPDYA (SEQ ID NO:142); FLAG (e.g., DYKDDDDK (SEQ ID NO:143); c-myc (e.g., EQKLISEEDL; SEQ ID NO:144), and the like.

### Affinity Domain

Affinity domains include peptide sequences that can interact with a binding partner, e.g., such as one immobilized on a solid support, useful for identification or purification. DNA sequences encoding multiple consecutive single amino acids, such as histidine, when fused to the expressed protein, may be used for one-step purification of the recombinant protein by high affinity binding to a resin column, such as nickel sepharose. Exemplary affinity domains include His5 (HHHHH) (SEQ ID NO:145), HisX6 (HHHHHH) (SEQ ID NO:146), C-myc (EQKLISEEDL) (SEQ ID NO:144), Flag (DYKDDDDK) (SEQ ID NO:143), StrepTag (WSHPQFEK) (SEQ ID NO:147), hemagglutinin, e.g., HA Tag (YPYDVPDYA) (SEQ ID NO:142), GST, thioredoxin, cellulose binding domain, RYIRS (SEQ ID NO:148), Phe-His-His-Thr (SEQ ID NO:149), chitin binding domain, S-peptide, T7 peptide, SH2 domain, C-end RNA tag, WEAAAREACCRECCARA (SEQ ID NO:150), metal binding domains, e.g., zinc binding domains or calcium binding domains such as those from calcium-binding proteins, e.g., calmodulin, troponin C, calcineurin B, myosin light chain, recoverin, S-modulin, visinin, VILIP, neurocalcin, hippocalcin, frequenin, caltractin, calpain large-subunit, S100 proteins, parvalbumin, calbindin D9K, calbindin D28K, and calretinin, inteins, biotin, streptavidin, MyoD, Id, leucine zipper sequences, and maltose binding protein.

### Detectable Signal-Producing Polypeptides

Suitable detectable signal-producing proteins include, e.g., fluorescent proteins; enzymes that catalyze a reaction that generates a detectable signal as a product; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilised ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

### Nucleic Acids

The present disclosure provides a nucleic acid that comprises a nucleotide sequence encoding a heteromeric, conditionally repressible synthetic ICR of the present disclosure. A nucleic acid comprising a nucleotide sequence encoding heteromeric, conditionally repressible synthetic ICR of the present disclosure will in some embodiments be DNA, including, e.g., a recombinant expression vector. A nucleic acid comprising a nucleotide sequence encoding heteromeric, conditionally repressible synthetic ICR of the present disclosure will in some embodiments be RNA, e.g., in vitro synthesized RNA.

In some cases, a nucleic acid of the present disclosure comprises a nucleotide sequence encoding only a first portion, e.g., a first part of a heteromeric, conditionally repressible synthetic ICR of the present disclosure. In some cases, a nucleic acid of the present disclosure comprises a nucleotide sequence encoding only a second portion, e.g., a second part, of a heteromeric, conditionally repressible synthetic ICR of the present disclosure. In some cases, a nucleic acid of the present disclosure comprises a nucleotide sequence encoding all or both parts of a heteromeric, conditionally repressible synthetic ICR of the present disclosure.

In some instances, a nucleic acid of the present disclosure comprises a nucleotide sequence encoding only the synthetic ICR repressor of a heteromeric, conditionally repressible synthetic ICR of the present disclosure. In some instances, a nucleic acid of the present disclosure comprises a nucleotide sequence encoding only the synthetic stimulatory ICR of a heteromeric, conditionally repressible synthetic ICR of the present disclosure.

In some cases, a subject nucleic acid provides for production of a heteromeric, conditionally repressible synthetic ICR of the present disclosure, e.g., in a mammalian cell. In other cases, a subject nucleic acid provides for amplification of the heteromeric, conditionally repressible synthetic ICR-encoding nucleic acid.

A nucleotide sequence encoding a heteromeric, conditionally repressible synthetic ICR of the present disclosure can be operably linked to a transcriptional control element, e.g., a promoter, and enhancer, etc. In some instances, the heteromeric, conditionally repressible synthetic ICR encoding nucleic acid is operably linked to a tissue specific promoter for expression in a particular cell type of interest. For example, a heteromeric, conditionally repressible synthetic ICR may be operably linked to an immune cell specific promoter for specific expression in one or more immune cell types. In other instances, a heteromeric, conditionally repressible synthetic ICR may be operably linked to a general (i.e., non-immune cell specific) promoter including e.g., a ubiquitous promoter, a constitutive promoter, a heterologous promoter, a regulatable promoters (e.g., inducible promoters, reversible promoters, etc.), etc.

### General Promoters

Suitable promoter and enhancer elements are known in the art. For expression in a bacterial cell, suitable promoters include, but are not limited to, lacI, lacZ, T3, T7, gpt, lambda P and trc. For expression in a eukaryotic cell, suitable promoters include, but are not limited to; cytomegalovirus immediate early promoter; herpes simplex virus thymidine kinase promoter; early and late SV40 promoters; promoter present in long terminal repeats from a retrovirus; mouse metallothionein-I promoter; and various art-known promoters.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol., 1991: 173(1): 86-93; Alpuche-Aranda et al., PNAS, 1992; 89(21): 10079-83), a nirB promoter (Harborne et al. (1992) Mol. Micro. 6:2805-2813), and the like (see, e.g., Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al. (2002) Infect. Immun. 70:1087-1096); an rpsM promoter (see, e.g., Valdivia and Falkow (1996). Mol. Microbiol. 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al. (1984) Nucl. Acids Res. 12:7035); and the like. Suitable strong promoters for use in prokaryotes such as Escherichia coli include, but are not limited to Trc, Tac, T5, T7, and P_{Lambda}. Non-limiting examples of operators for use in bacterial host cells include a lactose promoter operator (LacI repressor protein changes conformation when contacted with lactose, thereby preventing the LacI repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25).

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, e.g., for expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHO5 promoter, a CUP1 promoter, a GAL7 promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art.

### Immune Cell Promoters

In some instances, nucleic acids of the present disclosure include immune cell specific promoters that are expressed in one or more immune cell types, including but not limited to lymphocytes, hematopoietic stem cells and/or progeny thereof (i.e., immune cell progenitors), etc. Any convenient and appropriate promoter of an immune cell specific gene may find use in nucleic acids of the present disclosure. In some instances, an immune cell specific promoter of a nucleic acid of the present disclosure may be a T cell specific promoter. In some instances, an immune cell specific promoter of a nucleic acid of the present disclosure may be a light and/or heavy chain immunoglobulin gene promoter and may or may not include one or more related enhancer elements.

In some instances, an immune cell specific promoter of a nucleic acid of the present disclosure may be a promoter of a B29 gene promoter, a CD14 gene promoter, a CD43 gene promoter, a CD45 gene promoter, a CD68 gene promoter, a IFN-β gene promoter, a WASP gene promoter, a T-cell receptor β-chain gene promoter, a V9 γ (TRGV9) gene promoter, a V2 δ (TRDV2) gene promoter, and the like.

In some instances, an immune cell specific promoter of a nucleic acid of the present disclosure may be a viral promoter expressed in immune cells. As such, in some instances, viral promoters useful in nucleic acids of the present disclosure include viral promoters derived from immune cells viruses, including but not limited to, e.g., lentivirus promoters (e.g., HIV, SIV, FIV, EIAV, or Visna promoters) including e.g., LTR promoter, etc., Retroviridae promoters including, e.g., HTLV-I promoter, HTLV-II promoter, etc., and the like.

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90:7739; and Marodon et al. (2003) Blood 101:3416. As another example, a CD8 gene promoter can be used. NK cell-specific expression can be achieved by use of an Ncr1 (p46) promoter; see, e.g., Eckelhart et al. (2011) Blood 117:1565.

### Additional Nucleic Acid Components, Constructs and Use Thereof

In some instances, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., PNAS (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. (2006) Annual Review of Biochemistry, 567-605 and Tropp (2012) Molecular Biology (Jones & Bartlett Publishers, Sudbury, MA), the disclosures of which are incorporated herein by reference.

A nucleotide sequence encoding a subject conditionally repressible ICR can be present in an expression vector and/or a cloning vector. Where a subject conditionally repressible ICR is split between two or more separate polypeptides, nucleotide sequences encoding the two or more polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Large numbers of suitable vectors and promoters are known to those of skill in the art; many are commercially available for generating a subject recombinant constructs. The following vectors are provided by way of example. Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641648, 1999; Ali et al., Hum Mol Genet 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

As noted above, in some embodiments, a nucleic acid comprising a nucleotide sequence encoding a heteromeric, conditionally repressible synthetic ICR of the present disclosure will in some embodiments be RNA, e.g., in vitro synthesized RNA. Methods for in vitro synthesis of RNA are known in the art; any known method can be used to synthesize RNA comprising a nucleotide sequence encoding the first and/or the second polypeptide of a heteromeric, conditionally repressible synthetic ICR of the present disclosure. Methods for introducing RNA into a host cell are known in the art. See, e.g., Zhao et al. (2010) Cancer Res. 15:9053. Introducing RNA comprising a nucleotide sequence encoding the first and/or the second polypeptide of a heteromeric, conditionally repressible synthetic ICR of the present disclosure into a host cell can be carried out in vitro or ex vivo or in vivo. For example, a host cell (e.g., an NK cell, a cytotoxic T lymphocyte, etc.) can be electroporated in vitro or ex vivo with RNA comprising a nucleotide sequence encoding the first and/or the second polypeptide of a heteromeric, conditionally repressible synthetic ICR of the present disclosure.

### Cells

The present disclosure provides a mammalian cell that is genetically modified to produce a heteromeric, conditionally repressible synthetic ICR of the present disclosure.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, Hut-78, Jurkat, HL-60, NK cell lines (e.g., NKL, NK92, and YTS), and the like.

In some instances, suitable cells include those described in Themeli et al. Cell Stem Cell. 2015 Apr. 2; 16(4):357-66; the disclosure of which is incorporated herein by reference in its entirety.

In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a T lymphocyte, or progenitor thereof, obtained from an individual. As another example, the cell is a cytotoxic cell, or progenitor thereof, obtained from an individual. As another example, the cell is a stem cell or progenitor cell obtained from an individual.

### Methods of Modulating Immune Cell Activation

The present disclosure provides methods of repressing immune cell activation, such methods being applicable in vitro, in vivo, or ex vivo. The methods generally involve contacting an immune cell (in vitro, in vivo, or ex vivo) with a dimerizing agent, where the immune cell is genetically modified to produce a heteromeric, conditionally repressible synthetic ICR of the present disclosure. In the presence of the dimerizing agent, the heteromeric, conditionally repressible ICR dimerizes and represses activation of the immune cell, thereby producing a repressed immune cell. Immune cells include, e.g., a cytotoxic T lymphocyte, an NK cell, a CD4⁺ T cell, a T regulatory (Treg) cell, etc.

Contacting the genetically modified immune cell (e.g., a T lymphocyte, an NK cell) with a dimerizing agent can repress the expression of a lymphocyte cell surface antigen, e.g., a cell surface antigen indicative of immune cell activation, T cell activation, etc., by the immune cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of the cell surface antigen expressed by the activated immune cell in the absence of the dimerizing agent. Lymphocyte cell surface antigens whose production can be repressed include, but are not limited to e.g., CD69.

Contacting the genetically modified immune cell (e.g., a T lymphocyte, an NK cell) with a dimerizing agent can repress the production and/or secretion of a cytokine by the immune cell by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared with the amount of cytokine produced by the activated immune cell in the absence of the dimerizing agent. Cytokines whose production can be repressed include, but are not limited to, IL-2 and IFN-7.

Contacting the genetically modified immune cell (e.g., a T lymphocyte, an NK cell) with a dimerizing agent can repress proliferation of the immune cell. The amount of repression of proliferation may vary and may, in some instances, be about 5% or more, about 10% or more, about 15% or more, about 20% or more, about 25% or more, about 30% or more, about 40% or more, about 50% or more, about 75% or more, etc., as compared with the amount of proliferation seen in the activated immune cell in the absence of the dimerizing agent. Useful measures of immune cell proliferation include but are not limited to e.g., immune cell counts (e.g., as counted on a slide or measured by a cell counter or cytometric device (e.g. flow cytometer)), cell proliferation assays (e.g., as measured through the use of synthetic nucleoside (e.g., BrdU, EdU, etc.) incorporation; as measured using a proliferation dye (e.g., Horizon Violet, CFSE, etc.), etc.), expression of one or more cell cycle and/or proliferation markers (e.g., Ki-67, phosphohistone H3, proliferating cell nuclear antigen (PCNA), cyclins, cyclin-dependent kinases, retinoblastoma, etc.), and the like.

Contacting the genetically modified immune cell (e.g., a T lymphocyte, an NK cell) with a dimerizing agent can repress target cell killing by the immune cell. The amount of repression of target cell killing will vary and may, in some instances, be about 5% or more, about 10% or more, about 15% or more, about 20% or more, about 25% or more, about 30% or more, about 40% or more, about 50% or more, about 75% or more, etc., as compared with the amount of target cell killing seen in the activated immune cell in the absence of the dimerizing agent. Useful measures of target cell killing include but are not limited to e.g., target cell counts, (e.g., as counted on a slide or measured by a cell counter or cytometric device (e.g. flow cytometer)), target cell viability (e.g., as measured through the use of one or more viability stains or dyes (e.g., trypan blue, propidium iodide, BD Horizon violet/blue/red, etc.), expression of one or more cell death markers (e.g., activated caspase, phosphatidylserine exposure, mitochondria membrane potential, DNA fragmentation, expression or one or more cell death genes, expression or one or more cell death proteins, etc.), and the like.

### Methods of Generating a Conditionally Repressible Immune Cell

The present disclosure provides a method of generating a conditionally repressible cell. The method generally involves genetically modifying a mammalian cell with an expression vector, or an RNA (e.g., in vitro transcribed RNA), comprising nucleotide sequences encoding a heteromeric, conditionally repressible ICR of the present disclosure. The genetically modified cell is conditionally repressible in the presence of a dimerizer (a dimerizing agent). The genetic modification can be carried out in vivo, in vitro, or ex vivo. The cell can be an immune cell (e.g., a T lymphocyte or NK cell), a stem cell, a progenitor cell, etc.

In some cases, the genetic modification is carried out ex vivo. For example, a T lymphocyte, a stem cell, or an NK cell is obtained from an individual; and the cell obtained from the individual is genetically modified to express a conditionally repressible ICR of the present disclosure. The genetically modified cell is conditionally repressible in the presence of a dimerizer. In some cases, the genetically modified cell is modulated ex vivo. In other cases, the genetically modified cell is introduced into an individual (e.g., the individual from whom the cell was obtained); and the genetically modified cell is modulated in vivo, e.g., by administering to the individual a dimerizer.

Activation of the cell may be repressed by administration of the dimerizer before, during or after the cell is activated, e.g., by administering the antigen to the cell, by placing the cell in the presence of the antigen, by exposure of the cell to the antigen, e.g., by nature of the cell being introduced to a cell, in vivo or ex vivo, expressing the antigen. For example, where the antigen is present on the surface of a cell in the individual, there is no need to administer the antigen. The genetically modified cell comes into contact with the antigen present on the surface of a cell in the individual; and, upon administration to the individual of a dimerizer, activation of the genetically modified cell is repressed. For example, where the genetically modified cell is a T lymphocyte, the genetically modified cell can exhibit cytotoxicity toward a cell that presents an antigen on its surface to which the conditionally repressible ICR binds and such activation may be repressed by administration of the dimerizer.

### Methods of Modulating Treatment

The present disclosure provides various methods of modulating immune cell activation-based treatment using a heteromeric, conditionally repressible synthetic ICR described herein. Immune cell activation of an immune cell expressing a heteromeric, conditionally repressible synthetic ICR may be modulated, e.g., repressed, by administration of a dimerizer. For example, in some instances, therapy may be initiated by activation of an immune cell expressing a heteromeric, conditionally repressible synthetic ICR and subsequent to the activation, e.g., following the course of the therapy or following some adverse event, the therapy may be modulated by administering the dimerizer and repressing the activation attributable to the heteromeric, conditionally repressible synthetic ICR.

As such, repression of immune cell activation may be performed at any point before, during or after therapy with a conditionally repressible ICR. For example, in some instances, a dimerizer may be administered prior to a course of treatment but after a subject immune cell has been modified to express the conditionally repressible ICR, e.g., in order to prevent premature activation of the immune cell. In other instances, a dimerizer may be administered during a course of treatment to modulate the activation of the immune cell expressing the conditionally repressible ICR, e.g., to control immune cell populations, control the aggressiveness of therapy, coordinate the therapy with other therapeutic treatment, adjust treatment based on measured parameters, e.g., treatment responsiveness, side effects, etc. In other instances, a dimerizer may be administered following or at the end of a course of treatment, e.g., to end the treatment, to evaluate the treatment outcome in the absence of the immune cell activation, to prepare for the next course of treatment, to coordinate with subsequent treatments, etc.

In some instances, a heteromeric, conditionally repressible synthetic ICR described herein may be used in a treatment method where T cell activation due to the stimulatory part of the heteromeric, conditionally repressible synthetic ICR is inhibited in the case of an adverse event experienced by the subject. For example, as depicted in FIG. 17, part 1, treatment may be initiated by administration of an engineered T cell infusion and tumor cell killing is initiated. If during the course of treatment the subject experiences an adverse event the subject may be administered an effective amount of a dimerizer, e.g., rapalog, such that the first and second dimerizer domains dimerize inhibiting the T cell stimulatory function of the heteromeric, conditionally repressible synthetic ICR to reduce the severity of, eliminate or otherwise treat the adverse event. Subsequently following alleviation or reduction of the adverse event or at a predetermined time following the start of the administration of the dimerizer, administration of the dimerizer may be terminated allowing the first and second dimerizer domains to dissociate and the stimulatory part of the heteromeric, conditionally repressible synthetic ICR to re-activate of the T cells.

Administration of the dimerizer in response to an adverse event will vary depending on the particular adverse event and the severity of the adverse event. In some instances, the dimerizer may be administered in a single dose sufficient to repress the repressible ICR and upon reduced bioavailability, e.g., due to metabolism, of the dimerizer the repressive effect is terminated and the T cell can become reactivated. In other instances, the dimerizer may be administered in multiple doses or continuously and the repressive effect may be terminated by discontinuing administration of the dimerizer.

In some instances, a heteromeric, conditionally repressible synthetic ICR described herein may be used in a treatment method where optimal T cell activity is achieved by titrating the stimulatory and repressive functions of the repressible ICR. For example, as depicted in FIG. 17, part 2, treatment may be initiated by administration of an engineered T cell infusion and tumor cell killing is initiated at some level. Following initiation of treatment and at any point during treatment the level of T cell activation may be titrated by administering a dimerizer to repress the stimulatory function of the repressible ICR. Titration of the treatment will vary and will depend on the particular desired balance between activation and repression of T cell activation. For example, in some instances the treatment will be titrated to balance adverse events and/or undesirable side effects with primary treatment effectiveness, i.e., the treatment response for the primary treatment objective, e.g., tumor cell killing. Treatment titration may be performed once or multiple times during a course of treatment include, e.g., initial treatment titration, continuous treatment titration, predetermined or scheduled treatment titration (e.g., titration following regular or prescheduled diagnostic or prognostic procedures to assess treatment effectiveness).

### Formulations, Dosages, and Routes of Administration

As discussed above, treatment methods of the present disclosure include treatments involving modulation of an immune cell activation-based treatment through administering to an individual in need thereof an effective amount of a dimerizer agent that mediates dimerization of the heteromeric, conditionally repressible synthetic ICR.

An “effective amount” of a dimerizer agent is in some cases an amount that, when administered in one or more doses to an individual in need thereof, decreases the level of cytotoxic activity of a T lymphocyte expressing a subject repressible ICR by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the T lymphocyte in the absence of the dimerizing agent.

An “effective amount” of a dimerizer agent is in some cases an amount that, when administered in one or more doses to an individual in need thereof, decreases the level of cytotoxic activity of an NK cell expressing a subject repressible ICR by at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the cytotoxic activity of the NK cell in the absence of the dimerizing agent.

In some embodiments, an effective amount of a dimerizer is an amount that, when administered, in one or more doses, is effective to reduce a symptom or adverse event attributable to the activated repressible ICR, by at least about 5%, at least about 10%, at least about 15%, at least about 20%, at least about 25%, at least about 30%, at least about 40%, at least about 50%, at least about 60%, at least about 70%, at least about 80%, at least about 90%, or more, compared to the symptom or adverse event attributable to the activated repressible ICR in the absence of treatment with the dimerizer.

### Formulations

In the subject methods, a dimerizer can be administered to the host using any convenient means capable of resulting in the desired therapeutic effect or diagnostic effect, e.g., to modulate a conditionally repressible ICR present in cells within the host (i.e., to repress immune cell activation due to the conditionally repressible ICR). Thus, the dimerizer can be incorporated into a variety of formulations for therapeutic administration. More particularly, a dimerizer can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

In pharmaceutical dosage forms, a dimerizer can be administered in the form of their pharmaceutically acceptable salts, or they may also be used alone or in appropriate association, as well as in combination, with other pharmaceutically active compounds. The following methods and excipients are merely exemplary and are in no way limiting.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of a dimerizer adequate to achieve the desired state in the subject being treated.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

For oral preparations, a dimerizer can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

A dimerizer can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions comprising a dimerizer are prepared by mixing the dimerizer having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration; see also Chen (1992) Drug Dev Ind Pharm 18, 1311-54.

The term “unit dosage form,” as used herein, refers to physically discrete units suitable as unitary dosages for human and animal subjects, each unit containing a predetermined quantity a dimerizer calculated in an amount sufficient to produce the desired effect in association with a pharmaceutically acceptable diluent, carrier or vehicle. The specifications for a given dimerizer may depend on the particular dimerizer employed and the effect to be achieved, and the pharmacodynamics associated with each dimerizer in the host.

In some embodiments, a dimerizer is formulated in a controlled release formulation. Sustained-release preparations may be prepared using methods well known in the art. Suitable examples of sustained-release preparations include semipermeable matrices of solid hydrophobic polymers containing the dimerizer in which the matrices are in the form of shaped articles, e.g. films, microcapsules, microparticles, or nanoparticles. Examples of sustained-release matrices include polyesters, copolymers of L-glutamic acid and ethyl-L-glutamate, non-degradable ethylene-vinyl acetate, hydrogels, polylactides, degradable lactic acid-glycolic acid copolymers and poly-D-(−)-3-hydroxybutyric acid. Possible loss of biological activity may be prevented by using appropriate additives, by controlling moisture content and by developing specific polymer matrix compositions.

### Dosages

A suitable dosage can be determined by an attending physician or other qualified medical personnel, based on various clinical factors. As is well known in the medical arts, dosages for any one patient depend upon many factors, including the patient's size, body surface area, age, the particular dimerizer to be administered, sex of the patient, time, and route of administration, general health, and other drugs being administered concurrently. A dimerizer may be administered, e.g., to modulate a conditionally repressible ICR present in cells within the individual (i.e., to repress immune cell activation due to the conditionally repressible ICR), in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 g to 10 mg per kilogram of body weight per minute.

Those of skill will readily appreciate that dose levels can vary as a function of the specific dimerizer, the severity of the symptoms and the susceptibility of the subject to side effects. Preferred dosages for a given compound are readily determinable by those of skill in the art by a variety of means.

### Routes of Administration

A dimerizer is administered to an individual, e.g., to modulate a conditionally repressible ICR present in cells within the individual (i.e., to repress immune cell activation due to the conditionally repressible ICR), using any available method and route suitable for drug delivery, including in vivo and ex vivo methods, as well as systemic and localized routes of administration.

Conventional and pharmaceutically acceptable routes of administration include intratumoral, peritumoral, intramuscular, intratracheal, intracranial, subcutaneous, intradermal, topical application, intravenous, intraarterial, rectal, nasal, oral, and other enteral and parenteral routes of administration. Routes of administration may be combined, if desired, or adjusted depending upon the dimerizer and/or the desired effect. A dimerizer can be administered in a single dose or in multiple doses. In some embodiments, a dimerizer is administered orally. In some embodiments, a dimerizer is administered via an inhalational route. In some embodiments, a dimerizer is administered intranasally. In some embodiments, a dimerizer is administered locally. In some embodiments, a dimerizer is administered intratumorally. In some embodiments, a dimerizer is administered peritumorally. In some embodiments, a dimerizer is administered intracranially. In some embodiments, a dimerizer is administered intravenously.

The agent can be administered to a host using any available conventional methods and routes suitable for delivery of conventional drugs, including systemic or localized routes. In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intratumoral, peritumoral, and intravenous routes, i.e., any route of administration other than through the alimentary canal. Parenteral administration can be carried to effect systemic or local delivery of a dimerizer. Where systemic delivery is desired, administration typically involves invasive or systemically absorbed topical or mucosal administration of pharmaceutical preparations.

A dimerizer can also be delivered to the subject by enteral administration. Enteral routes of administration include, but are not necessarily limited to, oral and rectal (e.g., using a suppository) delivery.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as an adverse event or symptom related to the immune cell activation due to the repressible ICR. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

In some embodiments, a dimerizer is administered by injection and/or delivery, e.g., to a site in a brain artery or directly into brain tissue. A dimerizer can also be administered directly to a target site e.g., by direct injection, by implantation of a drug delivery device such as an osmotic pump or slow release particle, by biolistic delivery to the target site, etc.

### Combination Therapy

In some embodiments, a dimerizer is administered to coordinate immune activation therapy, e.g., as an adjuvant therapy, with a standard cancer therapy. For example, in some instances, a dimerizer may be administered to repress immune cell activation in preparation for a course of treatment including one or more standard cancer therapies. In other instances, a dimerizer may be administered following one or more standard cancer therapies in order to repress immune cell activation until a desired time following the therapy, e.g., when some measured parameter is reached, and the immune cell is to be activated. In other instances, a dimerizer may be administered during a standard cancer therapy, e.g., where immune cell activation is utilized as an adjuvant therapy, in response to an adverse event or side effect related to the immune cell activation therapy.

Standard cancer therapies include surgery (e.g., surgical removal of cancerous tissue), radiation therapy, bone marrow transplantation, chemotherapeutic treatment, antibody treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Suitable antibodies for use in cancer treatment include, but are not limited to, naked antibodies, e.g., trastuzumab (Herceptin), bevacizumab (Avastin™), cetuximab (Erbitux™) panitumumab (Vectibix™), Ipilimumab (Yervoy™), rituximab (Rituxan), alemtuzumab (Lemtrada™), Ofatumumab (Arzerra™), Oregovomab (OvaRex™), Lambrolizumab (MK-3475), pertuzumab (Perjeta™), ranibizumab (Lucentis™) etc., and conjugated antibodies, e.g., gemtuzumab ozogamicin (Mylortarg™), Brentuximab vedotin (Adcetris™), 90Y-labelled ibritumomab tiuxetan (Zevalin™), 131I-labelled tositumoma (Bexxar™), etc. Suitable antibodies for use in cancer treatment include, but are not limited to, antibodies raised against tumor-associated antigens. Such antigens include, but are not limited to, CD20, CD30, CD33, CD52, EpCAM, CEA, gpA33, Mucins, TAG-72, CAIX, PSMA, Folate-binding protein, Gangliosides (e.g., GD2, GD3, GM2, etc.), Le y, VEGF, VEGFR, Integrin alpha-V-beta-3, Integrin alpha-5-beta-1, EGFR, ERBB2, ERBB3, MET, IGF1R, EPHA3, TRAILR1, TRAILR2, RANKL, FAP, Tenascin, etc.

Biological response modifiers suitable for use in connection with the methods of the present disclosure include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) interferon-α; (7) interferon-γ; (8) colony-stimulating factors; (9) inhibitors of angiogenesis; and (10) antagonists of tumor necrosis factor.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation, therefore compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

### Subjects Suitable for Treatment

A variety of subjects are suitable for treatment with a subject method of treating cancer. Suitable subjects include any individual, e.g., a human or non-human animal who has cancer, who has been diagnosed with cancer, who is at risk for developing cancer, who has had cancer and is at risk for recurrence of the cancer, who has been treated with an agent other than a conditionally repressible ICR for the cancer and failed to respond to such treatment, or who has been treated with an agent other than a conditionally repressible ICR for the cancer but relapsed after initial response to such treatment.

Subjects suitable for treatment with a subject immunomodulatory method include individuals who have an autoimmune disorder; individuals who are organ or tissue transplant recipients; and the like; individuals who are immunocompromised; and individuals who are infected with a pathogen.

In some instances, a subject suitable for treatment with a subject method include a subject who was previously treated with a non-repressible ICR therapy and suffered an adverse event or considerable symptoms (i.e., side-effects) of the non-repressible ICR therapy. In some instances, a subject suitable for treatment with a subject method is a subject that stopped, including prematurely stopped treatment with a non-repressible ICR due to the presence of an adverse event or considerable symptoms (i.e., side-effects) due to the non-repressible ICR therapy.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure numbered 1-45 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A heteromeric, conditionally repressible synthetic immune cell receptor (ICR) comprising: a synthetic stimulatory ICR comprising a first member of a dimerization pair linked to the synthetic stimulatory ICR; and a synthetic ICR repressor comprising a second member of the dimerization pair linked to an intracellular inhibitory domain.

Aspect 2. The conditionally repressible synthetic ICR of Aspect 1, wherein the synthetic stimulatory ICR comprises an intracellular co-stimulatory domain.

Aspect 3. The conditionally repressible synthetic ICR of Aspect 2, wherein the intracellular co-stimulatory domain is selected from the group consisting of: 4-1BB (CD137), CD28, ICOS, OX-40, BTLA, CD27, CD30, GITR, and HVEM.

Aspect 4. The conditionally repressible synthetic ICR of any one of the preceding aspects, wherein the first member of a dimerization pair is linked intracellularly to the synthetic stimulatory ICR and the second member of the dimerization pair is linked intracellularly to the intracellular inhibitory domain.

Aspect 5. The conditionally repressible synthetic ICR of any one of the preceding aspects, wherein the synthetic ICR repressor further comprises a transmembrane domain.

Aspect 6. The conditionally repressible synthetic ICR of Aspect 5, wherein the second member of the dimerization pair is linked intracellularly to the transmembrane domain.

Aspect 7. The conditionally repressible synthetic ICR of Aspect 5, wherein the second member of the dimerization pair is extracellular and linked to the intracellular inhibitory domain by way of the transmembrane domain.

Aspect 8. The conditionally repressible synthetic ICR of any one of the preceding aspects, wherein the stimulatory ICR binds a soluble antigen.

Aspect 9. The conditionally repressible synthetic ICR of any one of the preceding aspects, wherein the stimulatory ICR binds a cell surface antigen.

Aspect 10. The conditionally repressible synthetic ICR of any one of the preceding aspects, wherein the stimulatory ICR binds a protein associated with the TCR complex.

Aspect 11. The conditionally repressible synthetic ICR of any one of the preceding aspects, wherein the intracellular inhibitory domain is an inhibitory domain derived from a protein selected from the group consisting of: PD-1, CTLA4, HPK1, SHP1, SHP2, Sts1 and Csk.

Aspect 12. The conditionally repressible synthetic ICR of any one of the preceding aspects, wherein the synthetic stimulatory ICR comprises an intracellular signaling domain selected from the group consisting of: a CD3-zeta signaling domain, a ZAP70 signaling domain and an immunoreceptor tyrosine-based activation motif (ITAM).

Aspect 13. The conditionally repressible synthetic ICR of any one of the preceding aspects, wherein the synthetic stimulatory ICR comprises an intracellular signaling domain that comprises a lymphocyte-specific protein tyrosine kinase (Lck) interaction sites.

Aspect 14. The conditionally repressible synthetic ICR of any one of the preceding aspects, wherein the first and second members of the dimerization pair form a homodimer in the presence of a small molecule dimerizer.

Aspect 15. The conditionally repressible synthetic ICR of any one of the preceding aspects, wherein the first and second members of the dimerization pair form a heterodimer in the presence of a small molecule dimerizer.

Aspect 16. The conditionally repressible synthetic ICR of any one of the preceding aspects, wherein the dimerization pair is a dimerization pair responsive to a small molecule selected from the group consisting of: rapamycin or an analog thereof, gibberellic acid or an analog thereof, coumermycin or an analog thereof, methotrexate or an analog thereof, abscisic acid or an analog thereof and tamoxifen or an analog thereof.

Aspect 17. The conditionally repressible synthetic ICR of any one of the preceding aspects, wherein the synthetic stimulatory ICR is a synthetic chimeric antigen receptor (CAR) or portion thereof.

Aspect 18. The conditionally repressible synthetic ICR of any one of the preceding aspects, wherein the synthetic stimulatory ICR is a synthetic T cell receptor (TCR) or portion thereof.

Aspect 19. The conditionally repressible synthetic ICR of any one of the preceding aspects, wherein the synthetic stimulatory ICR is a T cell-antigen coupler (TAC) or portion thereof.

Aspect 20. A mammalian cell genetically modified to produce the heteromeric, conditionally repressible synthetic ICR of any of the preceding aspects.

Aspect 21. The cell of Aspect 20, wherein the cell is a T cell.

Aspect 23. The cell of Aspect 21, wherein the cell is a CD4 T cell.

Aspect 24. The cell of Aspect 21, wherein the cell is a CD8 T cell.

Aspect 25. A nucleic acid comprising a nucleotide sequence encoding the heteromeric, conditionally repressible synthetic ICR of any one of Aspects 1 to 19.

Aspect 26. The nucleic acid of Aspect 25, wherein the nucleotide sequence is operably linked to a T cell specific promoter or a regulatable promoter.

Aspect 27. A recombinant expression vector comprising the nucleic acid of Aspect 25 or 26.

Aspect 28. The nucleic acid of Aspect 25, wherein the nucleic acid is in vitro transcribed RNA.

Aspect 29. A method of repressing T cell activation, the method comprising: contacting a T cell that expresses a heteromeric, conditionally repressible synthetic ICR of any one of Aspects 1-19 and has been activated by binding of an antigen or epitope to the synthetic stimulatory ICR with a dimerizing agent; wherein, in the presence of the dimerizing agent, the first and second members of the dimerization pair dimerize and the intracellular inhibitory domain represses the activation of the T cell.

Aspect 30. The method of aspect 29, wherein said contacting occurs in vivo.

Aspect 31. A method of making the cell of any of Aspects 20 to 24, the method comprising genetically modifying a mammalian cell with an expression vector comprising nucleotide sequences encoding the conditionally repressible synthetic ICR of any one of Aspects 1 to 19, or genetically modifying a mammalian cell with an RNA comprising nucleotide sequences encoding the conditionally repressible synthetic ICR of any one of Aspects 1 to 19.

Aspect 32. The method of Aspect 31, wherein said genetic modification is carried out ex vivo.

Aspect 33. The method of Aspect 31 or 32, wherein the cell is a T lymphocyte, a stem cell, an NK cell, a progenitor cell, a cell derived from a stem cell, or a cell derived from a progenitor cell.

Aspect 34. A method of modulating treatment of a cancer in an individual, the method comprising: genetically modifying an immune cell or immune cell progenitor obtained from the individual with an expression vector comprising nucleotide sequences encoding the conditionally repressible synthetic ICR of any one of Aspects 1 to 19, wherein the synthetic stimulatory ICR is specific for an epitope on a cancer cell in the individual; treating the individual with the genetically modified immune cell, immune cell progenitor or progeny thereof under conditions sufficient for killing of the cancer cell; and modulating the treatment of the individual by administering to the individual an effective amount of a dimerizing agent, wherein the dimerizing agent induces dimerization of the first and second members of the dimerization pair, wherein said dimerization provides for repression of the genetically modified immune cell, immune cell progenitor or progeny thereof.

Aspect 35. The method of Aspect 34, wherein the genetic modification is carried out ex vivo and the treating comprises introducing the genetically modified immune cell, immune cell progenitor or progeny thereof into the individual.

Aspect 36. A method of repressing the activity of a host cell, the method comprising contacting an activated host cell with a dimerizing agent, wherein the host cell is genetically modified to produce a conditionally repressible synthetic ICR of any one of Aspects 1 to 19, and wherein, in the presence of the dimerizing agent the first and second dimerizing members of the conditionally repressible synthetic ICR dimerize and represses at least one activity of the activated host cell.

Aspect 37. The method of Aspect 36, wherein the activity is selected from the group consisting of: proliferation, cell survival, apoptosis, gene expression, immune activation and combinations thereof.

Aspect 38. A heteromeric, conditionally repressible synthetic chimeric antigen receptor (CAR) comprising: a synthetic stimulatory CAR comprising: i) a extracellular recognition domain; ii) a transmembrane domain linked to the extracellular recognition domain; iii) a first member of a dimerization pair linked to the transmembrane domain; and iv) an intracellular stimulation domain; and a synthetic CAR repressor comprising: i) a second member of the dimerization pair; and ii) an intracellular inhibitory domain linked to the second member of the dimerization pair.

Aspect 39. The heteromeric, conditionally repressible synthetic CAR of Aspect 38, wherein the synthetic CAR repressor further comprises a transmembrane domain linked to the second member of the dimerization pair, the intracellular inhibitory domain or both.

Aspect 40. A heteromeric, conditionally repressible synthetic T cell receptor (TCR) comprising: a synthetic stimulatory TCR comprising: i) a transmembrane domain; ii) a first member of a dimerization pair linked to the transmembrane domain; iii) an engineered TCR polypeptide comprising at least one TCR alpha or beta chain, wherein the at least one TCR alpha or beta chain is linked to the transmembrane domain or the first member of a dimerization pair; and a synthetic TCR repressor comprising: i) a second member of the dimerization pair; and ii) an intracellular inhibitory domain linked to the second member of the dimerization pair.

Aspect 41. The heteromeric, conditionally repressible synthetic TCR of Aspect 40, wherein the synthetic TCR repressor further comprises a transmembrane domain linked to the second member of the dimerization pair, the intracellular inhibitory domain or both.

Aspect 42. The heteromeric, conditionally repressible synthetic TCR of Aspects 40 or 41, wherein the engineered TCR polypeptide further comprises a TCR gamma chain.

Aspect 43. A heteromeric, conditionally repressible T cell-antigen coupler (TAC) comprising: a synthetic stimulatory TAC comprising: i) a TCR specific binding domain; ii) a transmembrane domain; iii) a intracellular signaling domain; and iv) a first member of a dimerization pair; and a synthetic TAC repressor comprising: i) a second member of the dimerization pair; and ii) an intracellular inhibitory domain linked to the second member of the dimerization pair.

Aspect 44. The heteromeric, conditionally repressible TAC of Aspect 43, wherein the synthetic stimulatory TAC further comprises a target-specific binding domain.

Aspect 45. The heteromeric, conditionally repressible TAC of Aspect 43 or 44, wherein the synthetic TAC repressor further comprises a transmembrane domain linked to the second member of the dimerization pair, the intracellular inhibitory domain or both.

Aspect 46. The heteromeric, conditionally repressible TAC of any of Aspects 43-45, wherein the TCR specific binding domain specifically binds CD3.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Inhibition of Antigen-Induced T Cell Activation by Introduction of Rapalog Dimerizer to Conditionally Repressible Synthetic CAR Expressing T Cells

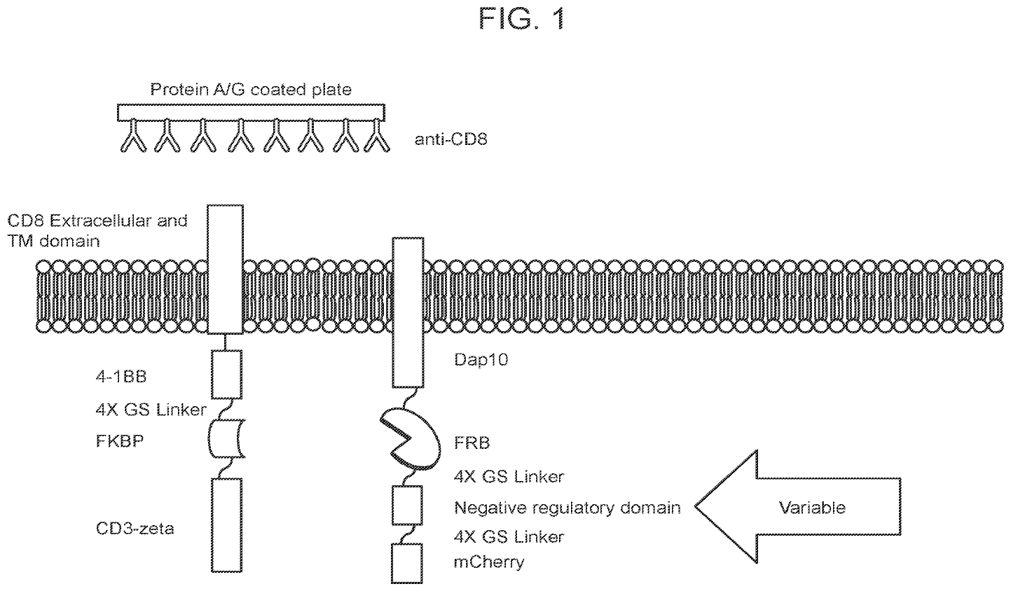

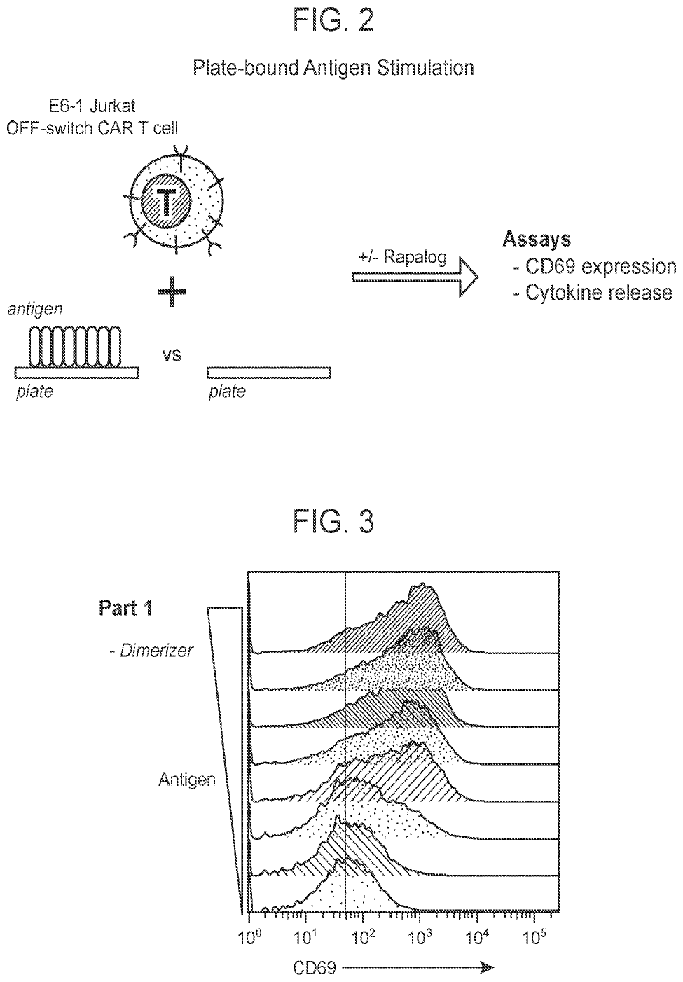

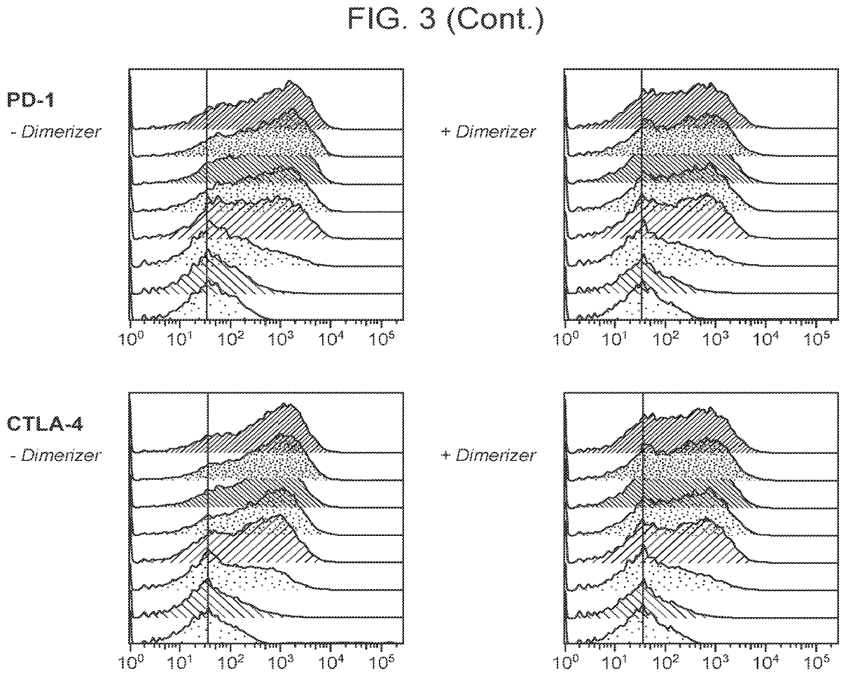

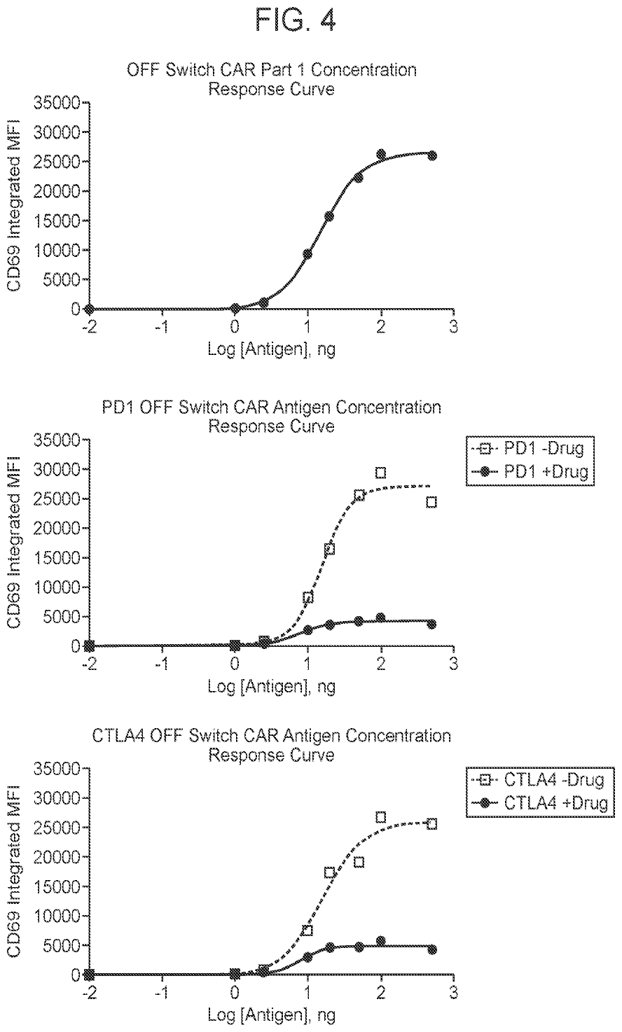

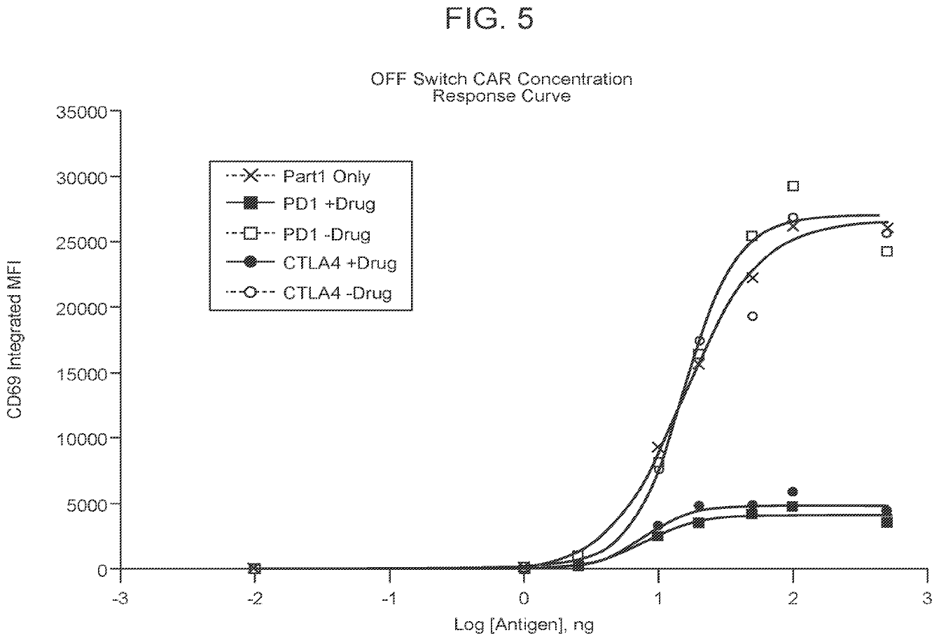

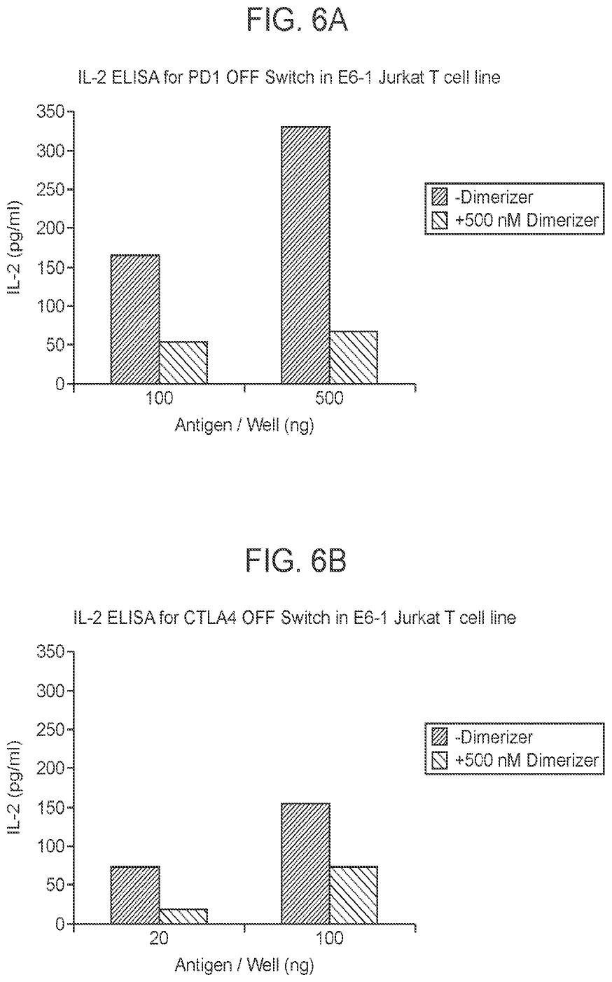

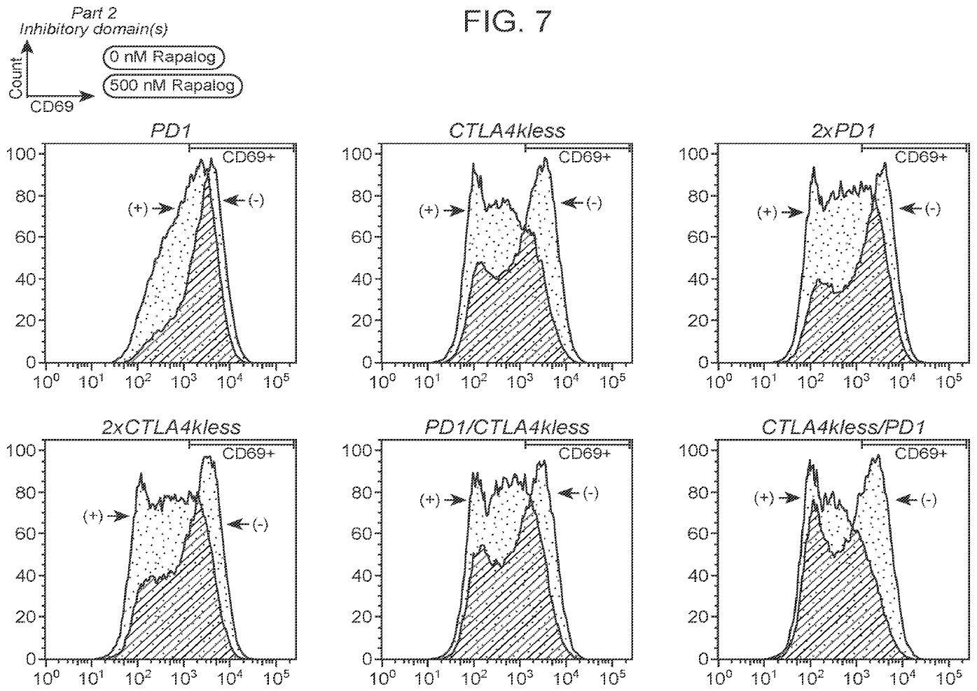

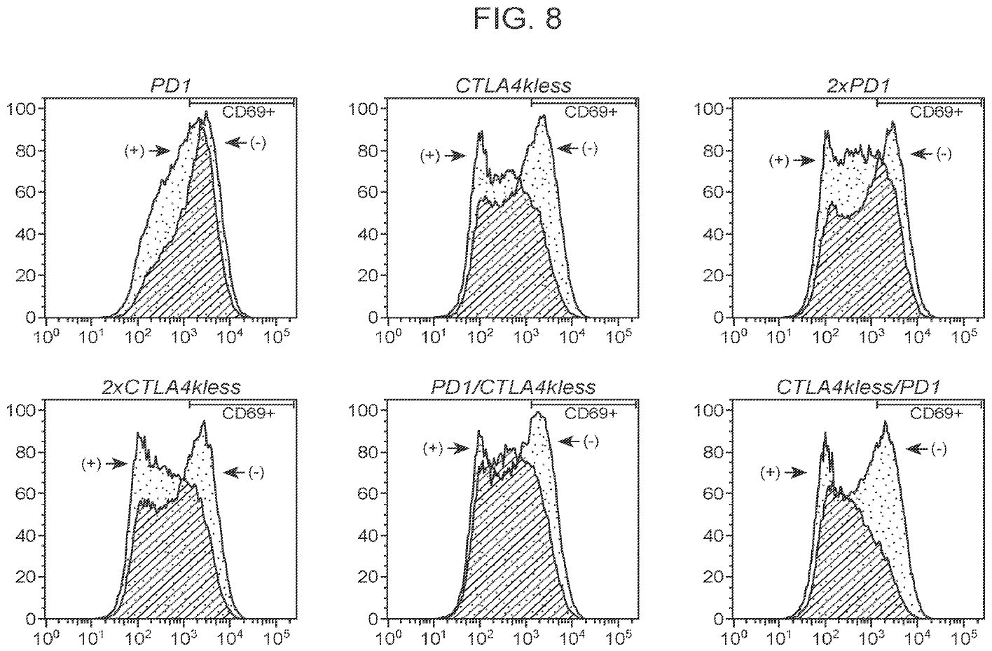

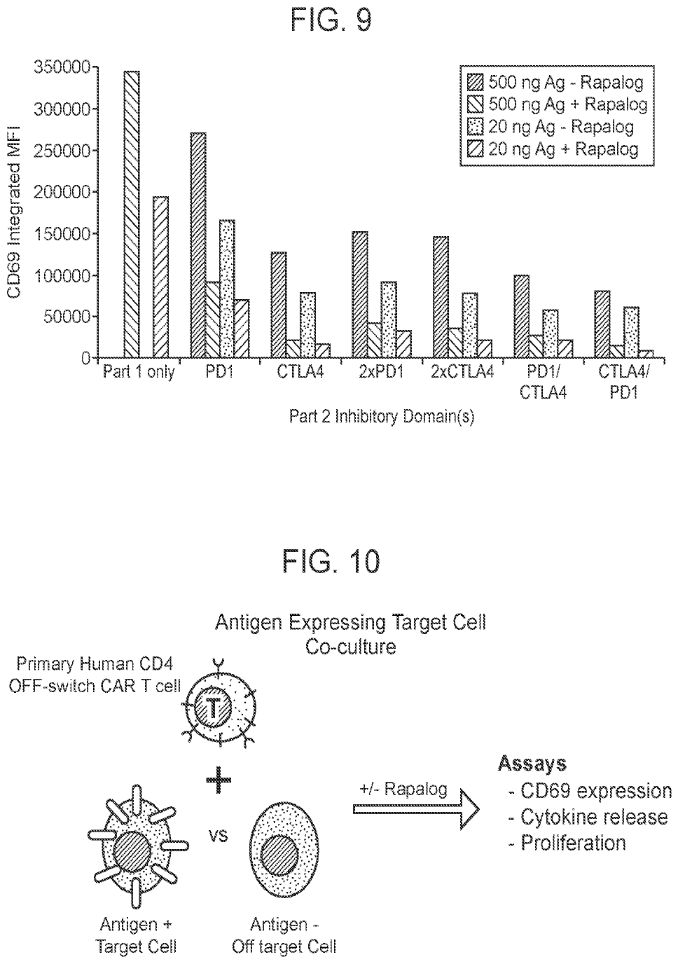

Heteromeric, conditionally repressible synthetic chimeric antigen receptors (CARs) having specificity for anti-CD8 were designed and constructed (FIG. 1). Synthetic CAR repressor domains were designed and constructed that contained various negative regulatory domains (cytoplasmic domain OFF-switches) including e.g., PD-1, CTLA4, HPK1, SHP1, SHP2, Sts1, and Csk. FKBP/FRB were used as the dimerizer pair. The conditionally repressible synthetic CARs (Part 1) were expressed with or without the synthetic CAR repressor domains (Part 2) and tested using E6-1 Jurkat T cells in a plate-bound antigen stimulation assay (FIG. 2). Cells were sorted for expression of the constructs by performing FACS with a BD FACSAria II cell sorter. Jurkat T cells expressing the OFF-switch CAR constructs were incubated overnight in wells coated with various amounts of plate-bound cognate antigen in the presence or absence of 500 nM rapalog. The rapalog was diluted in culture medium and added to the T cells at 500 nM 20 minutes prior to activation with antigen. T cell activation was monitored in single cells by quantifying expression of the cell surface protein CD69, a surface antigen up-regulated early during T cell activation. T cells in overnight assay mixtures were stained with a fluorophore-conjugated anti-CD69 antibody and analyzed by flow cytometry (FIG. 3, FIG. 4, FIG. 5, FIG. 7, FIG. 8, and FIG. 9). Supernatant from overnight assay mixtures was also collected and secretion of IL-2, a cytokine that is an indicator of an integrated cellular response, was quantified by ELISA (FIG. 6A-6B).

FIG. 3. Jurkat T cells expressing OFF-switch CAR constructs were incubated overnight in cell culture wells coated with various amounts of plate-bound cognate antigen in the presence or absence of 500 nM rapalog (see above). The OFF-switch CARs used in this experiment were as follows: (1) Part 1 Only, (2) Part 1+PD1 Part 2 and (3) Part 1+CTLA-4 Part2.

For each OFF-switch CAR, CD69 expression (x-axis, as determined by flow cytometry) is shown for various amounts of antigen (y-axis). CARs expressing Part 2 were tested with (+) and without (−) dimerizer. Increasing amount of antigen was shown to correlate with an increase in CD69 expression, indicating antigen mediated activation of the T cells. With the addition of the dimerizer drug, the OFF-switch constructs expressing PD1 or CTLA4 Part 2 showed a decrease in the number of T cells high activity state (measured by CD69 expression) (i.e. the number of cells shifted to high CD69 expression was reduced in the presence of dimerizer).

FIG. 4. Concentration responses curves show graphical depictions of the flow cytometry data presented in FIG. 3. When Jurkat T cells expressing the PD1 and CTLA4 OFF-switch constructs were stimulated with antigen in the absence of rapalog they display almost identical antigen-induced activation (“PD1-Drug” and “CTLA4-Drug”; see also overlay in FIG. 5). This finding indicates that, in Jurkat T cells, expression of Part 2 of the OFF-switch results in no inhibitory action in the absence of rapalog dimerizer. However, for both the PD1 and CTLA4 OFF-switch constructs addition of rapalog dimerizer was significantly reduced T cell activation over a range of cognate antigen levels (“PD1+Drug” and “CTLA4+Drug”).

Integrated mean fluorescence intensity (MFI; [(% MarkerX+)*(MarkerX mean fluorescence intensity)]) was used. Integrated MFI provides a quantitatively more correlative depiction of the functional response of cytokine-producing cells (See e.g., Shooshtari et al. Cytometry A. 2010; 77(9):873-80; the disclosure of which is incorporated herein by reference in its entirety).

FIG. 6A-6B. Jurkat T cells expressing the OFF-switch CAR constructs were incubated overnight in wells coated with various amounts of plate-bound cognate antigen in the presence or absence of 500 nM rapalog (see above). The OFF-switch CARs used in this experiment were as follows: (FIG. 6A) Part 1+PD1 Part 2 and (FIG. 6B) Part 1+CTLA4 Part2.

After overnight incubation, supernatant was collected, frozen, and later analyzed for IL-2 secretion by ELISA. For both the PD1 and CTLA4 OFF-switches, addition of 500 nM rapalog (dimerizer) was able to strongly inhibit IL-2 secretion at multiple levels of antigen stimulation. These findings demonstrate that CAR OFF-switches incorporating either PD1 or CTLA4 Part 2 constructs expressed in Jurkat T cells are able to inhibit antigen-induced T cell activation in a rapalog-dependent manner.

FIG. 7, E6-1 Jurkat OFF-Switch Part 2 Variations 500 ng Antigen Induced CD69 Expression. Jurkat T cells expressing the OFF-switch CAR constructs were incubated overnight in wells coated with 500 ng of plate-bound cognate antigen in the presence or absence of 500 nM rapalog (see above). The OFF-switch CARs used in this experiment were as follows: (1) Part 1 only; (2) Part 1+PD1 Part 2; (3) Part 1+CTLA4kless Part2; (4) Part 1+2×PD1 Part 2; (5) Part 1+2×CTLA4kless Part 2; (6) Part 1+PD1/CTLA4kless Part 2 and (7) Part 1+CTLA4kless/PD1 Part 2.

For each OFF-switch CAR, CD69 expression (determined by flow cytometry) is shown in the presence (+) and absence (−) of 500 nM rapalog. With the addition of the dimerizer drug all the OFF-switch constructs showed a decrease in the number of T cells in the CD69+ high activity state (as seen by a reduction in the number of cells shifted to high CD69 expression in the presence of dimerizer). This finding demonstrates, in part, that Part 2 of the OFF-switch CAR can function to repress T cell activation when constructed with single or multiple inhibitory domains.

FIG. 8, E6-1 Jurkat OFF-Switch Part 2 Variations 20 ng Antigen Induced CD69 Expression. Jurkat T cells expressing the OFF-switch CAR constructs were incubated overnight in wells coated with 20 ng of plate-bound cognate antigen in the presence or absence of 500 nM rapalog (see above). The OFF-switch CARs used in this experiment were as follows: (1) Part 1 only; (2) Part 1+PD1 Part 2; (3) Part 1+CTLA4kless Part2; (4) Part 1+2×PD1 Part 2; (5) Part 1+2×CTLA4kless Part 2; (6) Part 1+PD1/CTLA4kless Part 2 and (7) Part 1+CTLA4kless/PD1 Part 2.

With the addition of the dimerizer drug all the OFF-switch constructs show a decrease in the number of T cells in the CD69+ high activity state. This finding indicates that Part 2 of the OFF-switch CAR with single or multiple inhibitory domains can inhibit T cell activation induced by a range of cognate antigen levels.

FIG. 9. The CD69 integrated MFI for all of the histograms shown in FIG. 7 and FIG. 8 are displayed. Collectively, the data clearly show that at the two different antigen levels, CAR OFF-switches incorporating different inhibitory domain(s) into Part 2 are able to inhibit T cell activation (as measured by CD69 expression) in a drug-dependent manner.

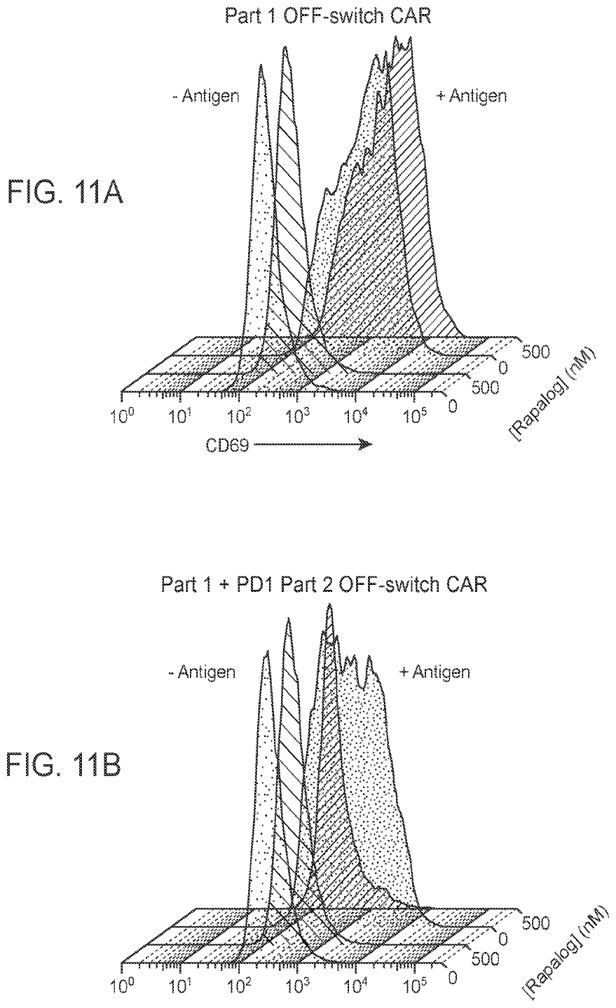

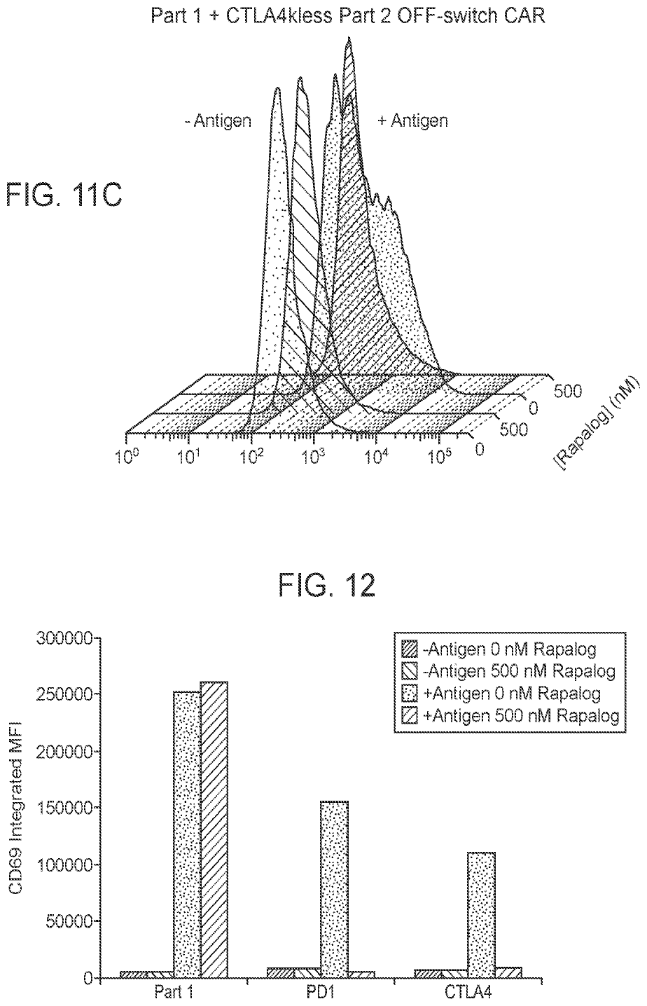

FIG. 10. Candidate OFF-switch CAR construct pairs (or Part 1 only) were expressed in primary human CD4 T cells. Cells were sorted for expression of the constructs by performing FACS with a BD FACSAria II cell sorter. Primary CD4 T cells expressing OFF-switch CARs were mixed with cognate (CD19+, “Antigen+Target Cell”) or non-cognate (mesothelin+, “Antigen—Off Target Cell”) K562 target cells at a 1:1 T cell:target cell ratio in a U-bottom 96-well plate. Dimerization was induced by incubation with rapalog diluted in culture medium and added to T cells at 500 nM 20 minutes prior to co-culture with target cells. Cell activation was assayed by various assays including, CD69 expression, cytokine release, and cell proliferation. T cells in overnight assay mixtures were stained with a fluorophore-conjugated anti-CD69 antibody and analyzed by flow cytometry (FIG. 11A-11C and FIG. 12)

FIG. 11A-11C. Primary human CD4 T cells expressing the OFF-switch CAR constructs were incubated overnight in wells coated with Antigen+ or Antigen-target cells in the presence or absence of 500 nM rapalog (see FIG. 10). The OFF-switch CARs used in this experiment were as follows: (1) Part 1 Only; (2) Part 1+PD1 Part 2 and (3) Part 1+CTLA4kless Part2.

For each OFF-switch CAR, CD69 expression (determined by flow cytometry) is shown for with target antigen (Antigen+) and off target antigen (Antigen-), as well as with (500 nM) and without (0 nM) rapalog dimerizer for both target cell types. No activation of T cells was seen when T cells containing any of the OFF-switches were incubated with off target antigen (Antigen-). When OFF-switch T cells were incubated with target antigen cells (Antigen+) in the absence of rapalog there was a large increase in CD69 expression for all OFF-switches. Addition of rapalog to the co-cultures with T cells expressing OFF-switch Part 1 only showed no effect on CD69 expression, indicating that the presence of rapalog alone (i.e., without Part 2) does not trigger the inhibitory effect. In contrast, addition of the rapalog to co-cultures with T cells expressing Part 1 and Part 2 (PD1 or CTLA4kless) of the OFF-switch caused a large reduction in the CD69 expression that was induced by target cell (antigen+) stimulation. These findings indicate that in primary human CD4 T cells, CAR OFF-switches incorporating either PD1 or CTLA4kless Part 2 constructs are able to inhibit antigen-induced T cell activation in a rapalog-dependent manner.

FIG. 12. The CD69 integrated MFI for all of the histograms shown in FIG. 11A-11C are graphed. In the case of both the PD1 and CTLA4kless OFF-switches, addition of the rapalog is able to strongly inhibit CD69 expression in response to cognate antigen sensing.

### Example 2: Alternative Designs for Heteromeric Conditionally Repressible Immune Cell Receptors

In certain embodiments, the general design for split OFF-Switch CAR and engineered T cell receptor (TCR) includes a Part 1 that activates the T cell upon ligand-binding and a Part 2 that is able to inhibit T cell activation upon drug-mediated dimerization. Various alternative designs for both Part 1 and Part 2 of the split OFF-Switch CARs and TCRs are contemplated, non-limiting examples of which are depicted in FIG. 13.

For Part 1, different potential ‘receptor bodies’ may be used, each of which is able to transduce binding of a target antigen into T cell activation, as well as different possible configurations of the heterodimerization domain. The ‘receptor bodies’ that will be incorporated into Part 1 are T cell immune activating receptors of various configurations that are modified with the addition of a heterodimerization domain, with or without the use of one or more linkers. In some instances, the ‘receptor bodies’ to which a heterodimerization domain are introduced include those described in, e.g., Grupp et al., N Engl J Med. 2013 (PMID: 23527958); Kalos et al., Sci Transl Med. 2011 (PMID: 21832238); Aggen et al., Gene Ther. 2012 (PMID: 21753797); Kochenderfer et al., Blood. 2010 (PMID: 20668228); Johnson et al., Blood. 2009 (PMID: 19451549); Sebesty et al., J Immunol. 2008 (PMID: 18490778); Morgan et al. S A, Rosenberg SA., Science. 2006 (PMID: 16946036); Schaft et al., Int Immunol. 2006 (PMID: 16507598); Zhao et al., J Immunol. 2005 (PMID: 15778407); Zhang et al., Cancer Gene Ther. 2004 (PMID: 15153936); Schaft et al., J Immunol Methods. 2003 (PMID: 12972184); Schaft et al., J Immunol. 2003 (PMID: 12574392); Willemsen et al., Gene Ther. 2000 (PMID: 10981663); Chung et al., Proc Natl Acad Sci USA. 1994 (PMID: 7809095); the disclosures of which are incorporated herein by reference in their entirety.

For designs that incorporate domains from the TCR, the constant domains of the alpha and beta TCR chains can be modified so as to improve engineered TCR expression and to reduce the expression of mispaired TCRs in which one of the engineered chains pairs with an endogenous TCR chain. For example, in some instances TCR modifications may include, e.g., murinized α+β chains, cysteine-modified α+β chains, domain-swapped α+β chains, and α+β chains with combinations of the modifications listed above as depicted in FIG. 14 and including but not limited to, e.g., those modifications described in Thomas et al. J Immunol. 2007; 179(9):5803-10 (PMID: 17947653); Cohen et al. Cancer Res. 2007; 67(8):3898-903 (PMID: 17440104); Kuball et al. Blood. 2007; 109(6):2331-8 (PMID: 17082316); Cohen et al. Cancer Res. 2006; 66(17):8878-86 (PMID: 16951205); Voss et al. Immunol Res. 2006; 34(1):67-87 (PMID: 16720899); Boulter et al. Protein Eng. 2003; 16(9):707-11 (PMID: 14560057); the disclosures of which are incorporated herein by reference in their entirety.

### Example 3: Titratable Inhibition of Activation in OFF-Switch CAR CD4 T Cells

The experimental setups and data collection used for all Primary T cell experiments described in Example 3 are as described above for and depicted in FIG. 10.

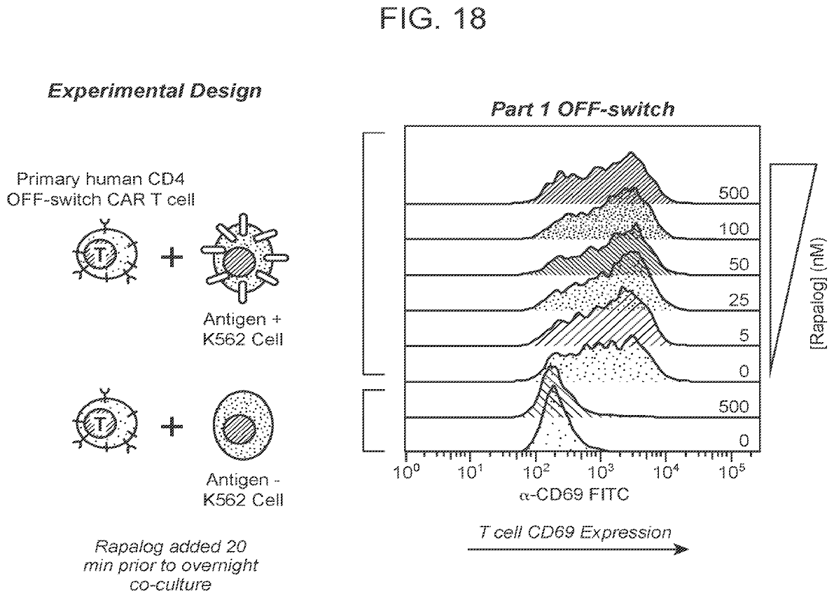

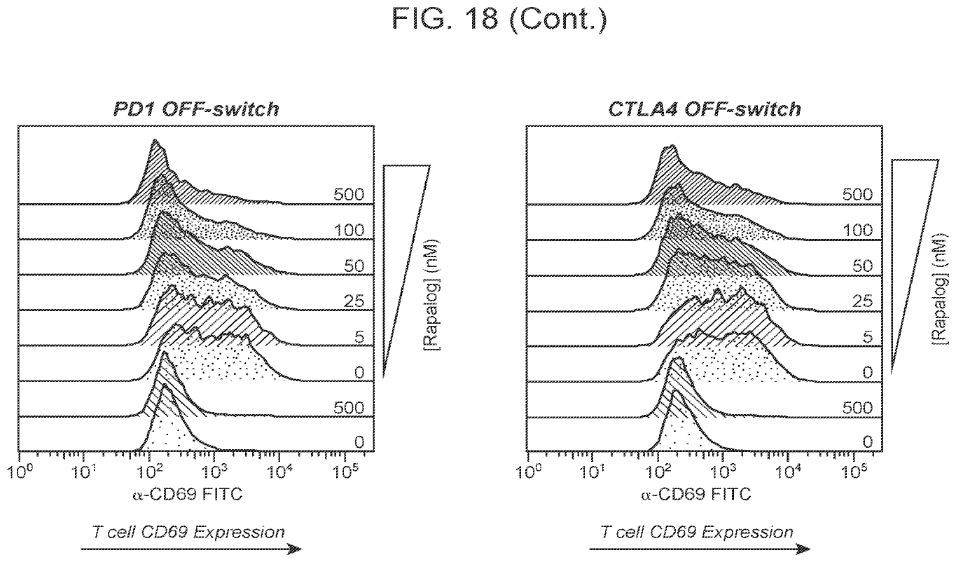

FIG. 18. CD69 Activation Marker Expression Rapalog Concentration Response.

Primary human CD4 T cells expressing OFF-switch CAR constructs were incubated overnight in wells together with Antigen+ or Antigen-target cells in the presence of 0, 5, 25, 50, 100, or 500 nM rapalog (see description of FIG. 10). The OFF-switch CARs used in this experiment were as follows: (1) Part 1 Only; (2) Part 1+PD1 Part 2 and (3) Part 1+CTLA4kless Part2.

For each OFF-switch CAR, CD69 expression (determined by flow cytometry) is shown for CAR T cells incubated with Antigen+ and Antigen− target cell types at varying concentrations of rapalog. When T cells containing the OFF-switches were incubated with Antigen− target cells, there was no activation of the T cells. When OFF-switch expressing T cells were incubated with Antigen+ target cells in the absence of rapalog there was a large increase in CD69 expression for all OFF-switches. Addition of rapalog to the co-cultures with T cells expressing OFF-switch Part 1 only had no effect on CD69 expression, indicating that the OFF-switch Part 2 is responsible for the inhibitory effect. In contrast, addition of the rapalog to co-cultures with T cells expressing the two parts of the OFF-switch (with PD1 or CTLA4kless Part 2) was able to cause a large reduction in CD69 expression induced by antigen+ target cell stimulation in a concentration-dependent manner, i.e. inhibition increased with increased rapalog concentration. These findings indicate that in primary human CD4 T cells, CAR OFF-switches incorporating either PD1 or CTLA4kless Part 2 constructs are able to inhibit antigen-induced T cell activation in a rapalog concentration-dependent manner.

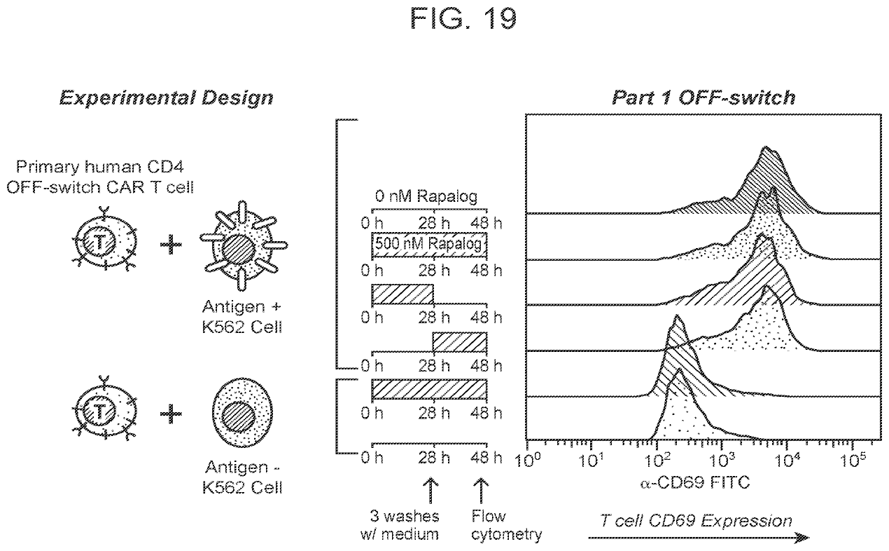

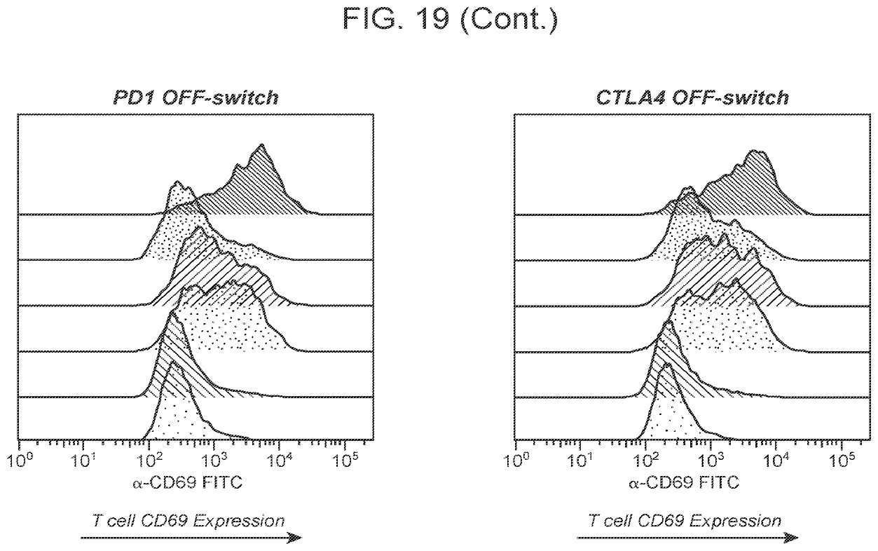

FIG. 19. CD69 Expression Varied Rapalog Timing.

Primary human CD4 T cells expressing the OFF-switch CAR constructs were incubated for 48 hours in wells together with Antigen+ or Antigen− target cells in the presence or absence of 500 nM rapalog (see FIG. 10 description). After 28 hours of co-culture, cells were wash three times with culture media and the media was replaced with media containing either the same or different rapalog concentration. The OFF-switch CARs used in this experiment were as follows: (1) Part 1 Only; (2) Part 1+PD1 Part 2 and (3) Part 1+CTLA4kless Part2.

For each OFF-switch CAR, CD69 expression (determined by flow cytometry) is shown for CAR T cells incubated with Antigen+ and Antigen− target cell types with different timing of rapalog dosing. When T cells containing any of the OFF-switches were incubated with Antigen− target cells, there was no activation of the T cells. When OFF-switch T cells were incubated with Antigen+ target cells in the absence of rapalog for the entire assay period there was a large increase in CD69 expression for all OFF-switches. Addition of rapalog at any time to the co-cultures with T cells expressing OFF-switch Part 1 only had no effect on CD69 expression, because Part 2 was not available for dimerization. The constant presence of 500 nM rapalog in co-cultures with T cells expressing the two parts of the OFF-switch (with PD1 or CTLA4kless Part 2) was able to cause a large reduction in CD69 expression induced by antigen+ target cell stimulation. The removal of rapalog after 28 hours allowed the PD1 and CTLA4kless OFF-switch T cells to activate as shown by CD69 upregulation, demonstrating the reversibility of the switch. The addition of rapalog after 28 hours induced inhibition of T cell activation in cells expressing PD1 or CTLA4kless Part 2 constructs. This finding indicates that OFF-switch CARs are able to inhibit already activated primary T cells.

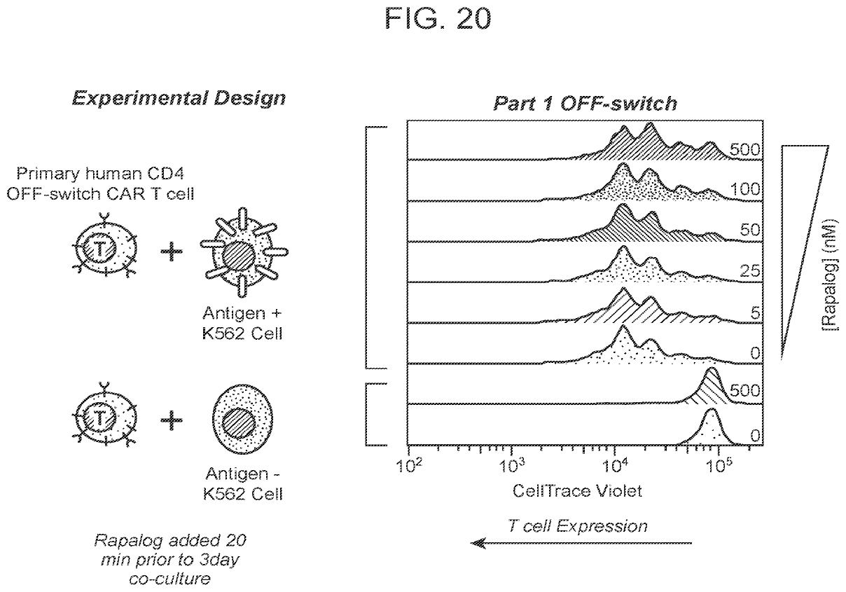

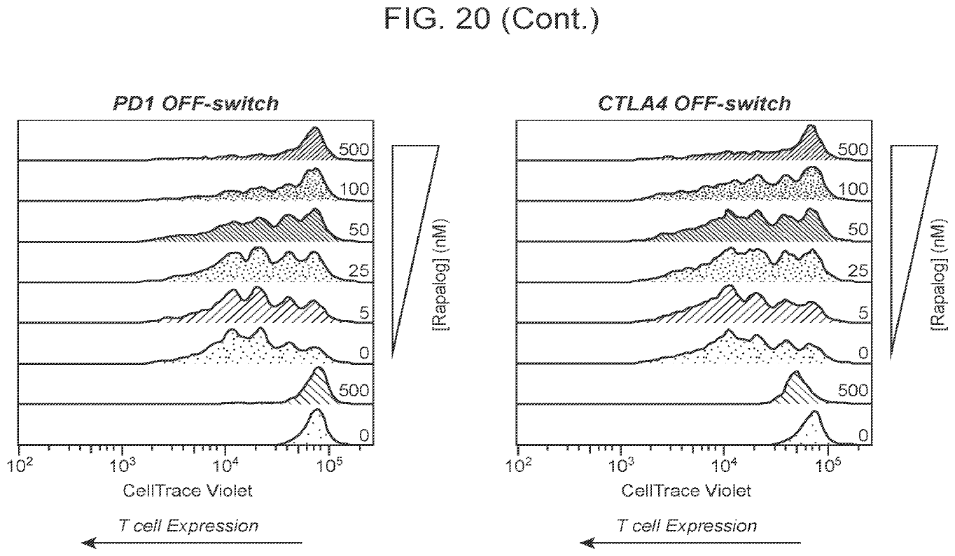

FIG. 20. T Cell Proliferation Rapalog Concentration Response.

Primary human CD4 T cells expressing the OFF-switch CAR constructs were incubated for 3 days in wells together with Antigen+ or Antigen− target cells in the presence of 0, 5, 25, 50, 100, or 500 nM rapalog (see FIG. 10 caption). The OFF-switch CARs used in this experiment were as follows: (1) Part 1 Only; (2) Part 1+PD1 Part 2 and (3) Part 1+CTLA4kless Part2.

For each OFF-switch CAR, CellTrace Violet dye staining (determined by flow cytometry) is shown for CAR T cells incubated with Antigen+ and Antigen− target cell types at varying concentrations of rapalog. CellTrace Violet was used to stain T cells prior to co-culture, and dilution of the dye (indicated by reduced fluorescence intensity) was used to track T cell proliferation. For both the PD1 and CTLA4kless OFF-switches, addition of increasing concentrations of rapalog led to corresponding reductions in T cell proliferation, a downstream result of T cell activation. These findings indicate that the PD1 and CTLA4kless OFF-switch CARs are able to inhibit primary CD4 T cell proliferation in a rapalog dose-dependent manner.

Primary human CD4 T cells expressing the OFF-switch CAR constructs were incubated overnight in wells together with Antigen(+) or Antigen(−) K562 target cells in the presence of 0, 25, 50, 100, or 500 nM rapalog. The OFF-switch CAR constructs used in this example were as follows: (1) Part 1 Only (“Part 1 OFF-switch); (2) Part 1+PD1 Part 2 (“PD1 OFF-switch”) and (3) Part 1+CTLA4kless Part2 (“CTLA4 OFF-Switch”).

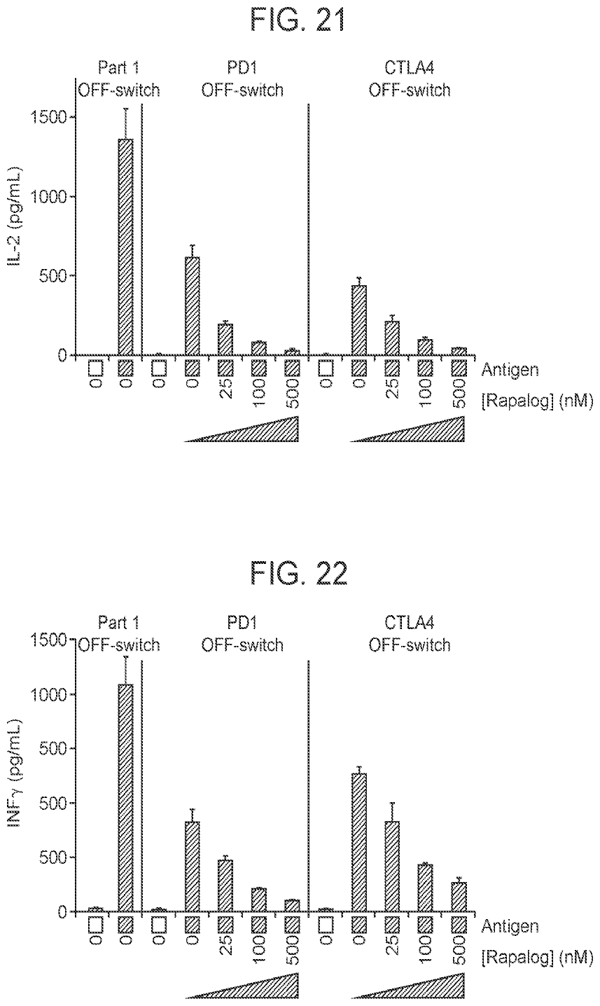

Results pertaining to titratable inhibition IL-2 cytokine secretion in this example are provided in FIG. 21. For each OFF-switch CAR, IL-2 secretion (as determined by Luminex assay) is shown for CAR T cells incubated with Antigen(+) or Antigen(−) target cell types at varying concentrations of rapalog. For both the PD1 and CTLA4kless OFF-switches, addition of increasing concentrations of rapalog led to corresponding reductions in IL-2 secretion, which is a downstream result of T cell activation. These findings indicate that the PD1 and CTLA4kless OFF-switch CARs are able to inhibit primary CD4 T cell IL-2 secretion in a rapalog dose-dependent manner.

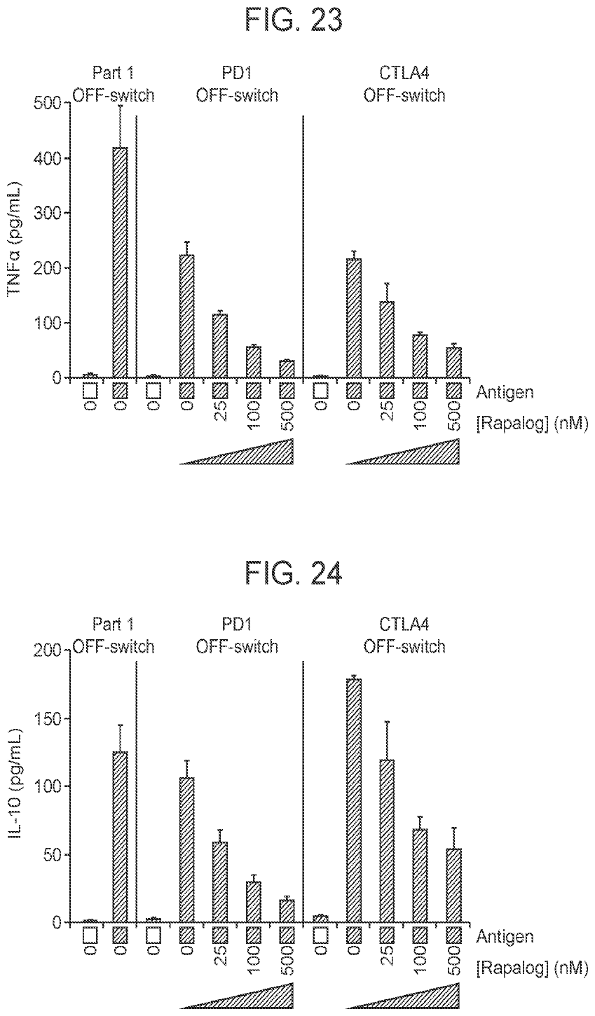

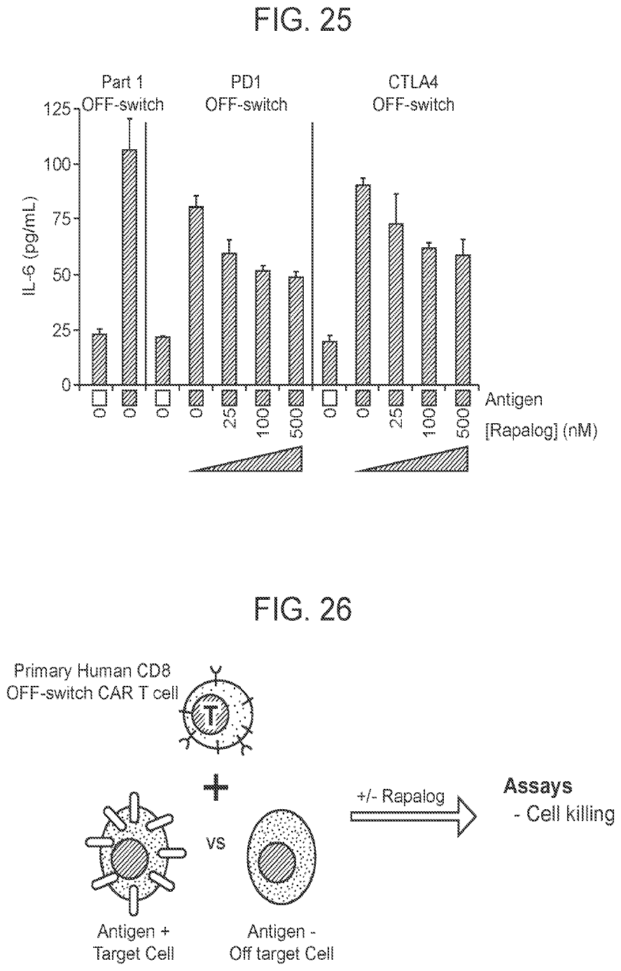

Titratable inhibition of activation of OFF-switch CAR CD4 T cells was also observed using INF-γ, INF-α, IL-10 and IL-6 readouts. Overnight co-culture was performed with K562 cells (antigen=CD19). Following co-culture supernatant was collected and INF-γ (FIG. 22), INF-α (FIG. 23), IL-10 (FIG. 24) and IL-6 (FIG. 25) were quantified by corresponding Luminex assay. In each assay, for both PD1 and CTLA4kless OFF-switches, addition of increasing concentrations of rapalog led to corresponding reductions in the measured readout of T cell activation. Collectively, these findings indicate that the PD1 and CTLA4kless OFF-switch CARs are able to inhibit activation of OFF-switch expressing T cells in a rapalog dose-dependent manner.

### Example 4: Titratable Cell Killing with OFF-Switch CAR CD8 Primary T Cells

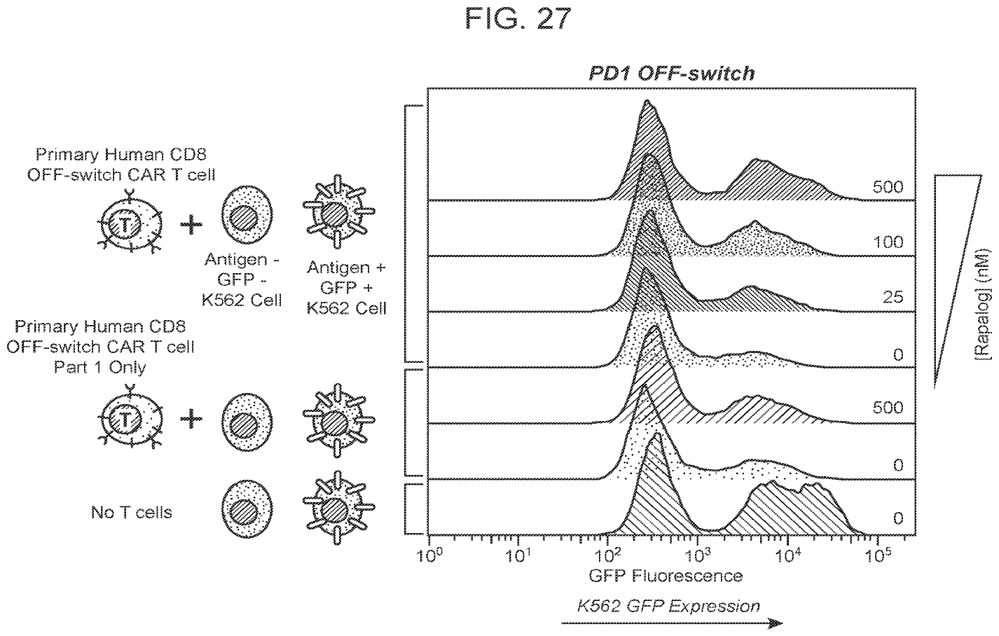

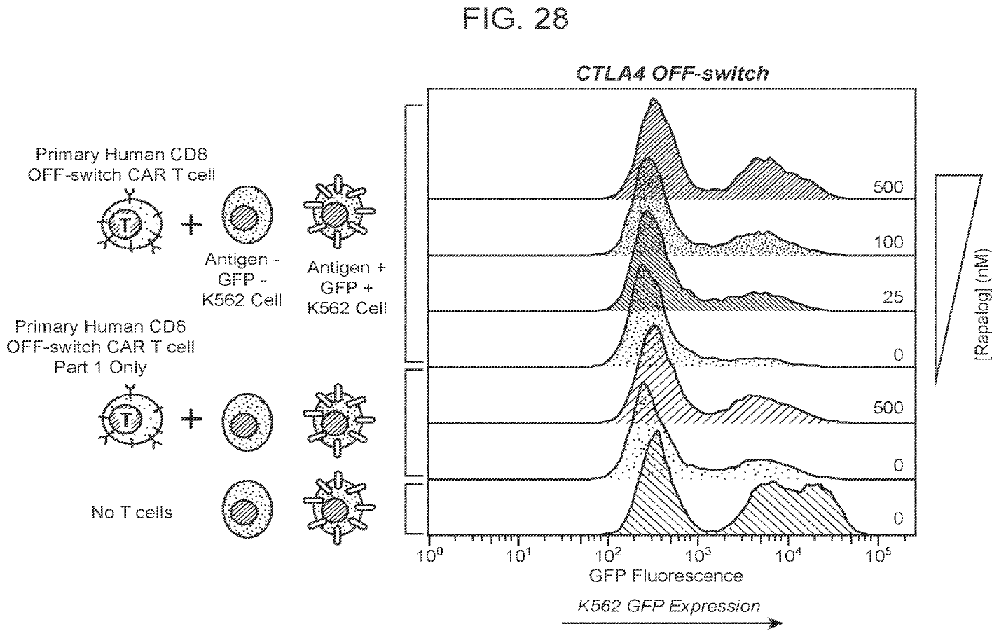

Primary human CD8 T cells expressing OFF-Switch CAR constructs were co-incubated with Antigen(+) or Antigen(−) K562 target cells in the presence of 0, 25, 50, 100, or 500 nM rapalog and cell killing was assessed using flow cytometry (FIG. 26). To assay cell killing specificity, OFF-switch CAR expressing CD8 T cells (PD1 or CTLA4) were co-incubated with both Antigen(+)/GFP(+) K562 target cells and Antigen(−)/GFP(−) K562 non-target cells. Dose dependent inhibition of specific cell killing was observed with both PD1 OFF-switch CAR expressing CD8 T cells (FIG. 27) and CTLA4 OFF-switch CAR expressing CD8 T cells (FIG. 28).

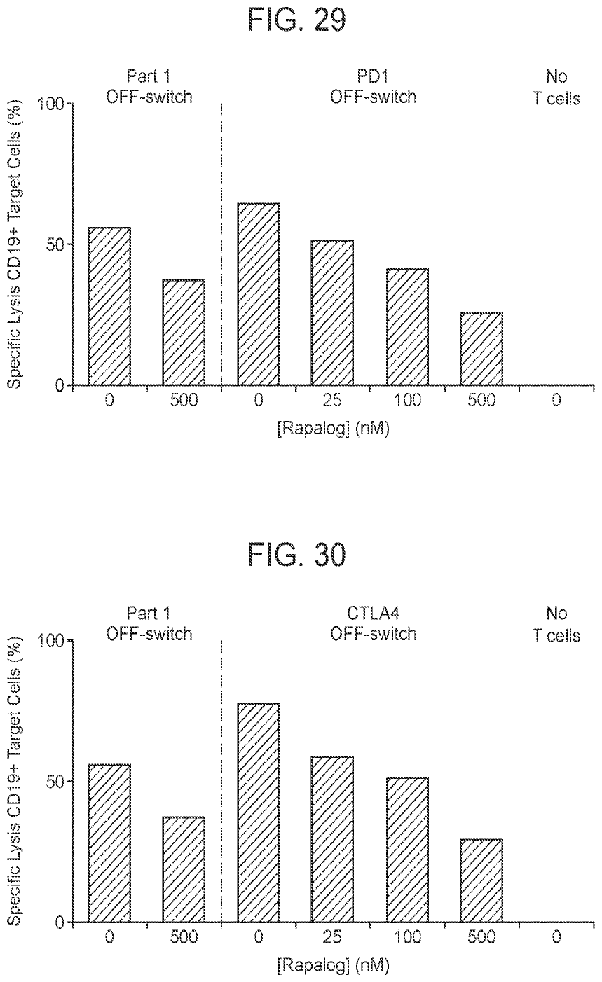

To quantify the specificity of cell killing a metric for specific lysis of CD19(+) Target cells was employed. Specific lysis was calculated as the difference between the percentage of GFP(+) cells in the control minus the percentage of GFP(+) in the respective experimental group divided by the percentage of GFP(+) cells in the control (i.e., (% GFP+con.−% GFP+condition)/% GFP+con.). The results of the lysis specificity calculations are provided in for both the PD-1 OFF-Switch CAR CD8 T cells (FIG. 29) and the CTLA4 OFF-Switch CAR CD8 T cells (FIG. 30).

Collectively, these data demonstrate that target cell specific killing attributable to OFF-Switch CAR expressing CD8 T cells is titratable in a rapalog dose-dependent manner.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A heteromeric, conditionally repressible synthetic immune cell receptor (ICR) comprising:
a synthetic stimulatory ICR comprising a first member of a dimerization pair linked to the synthetic stimulatory ICR; and
a synthetic ICR repressor comprising a second member of the dimerization pair linked to an intracellular inhibitory domain.

2. The conditionally repressible synthetic ICR of claim 1, wherein the synthetic stimulatory ICR comprises an intracellular co-stimulatory domain.

3. The conditionally repressible synthetic ICR of claim 2, wherein the intracellular co-stimulatory domain is selected from the group consisting of: 4-1BB (CD137), CD28, ICOS, OX-40, BTLA, CD27, CD30, GITR, and HVEM.

4. The conditionally repressible synthetic ICR of any one of the preceding claims, wherein the first member of a dimerization pair is linked intracellularly to the synthetic stimulatory ICR and the second member of the dimerization pair is linked intracellularly to the intracellular inhibitory domain.

5. The conditionally repressible synthetic ICR of any one of the preceding claims, wherein the synthetic ICR repressor further comprises a transmembrane domain.

6. The conditionally repressible synthetic ICR of claim 5, wherein the second member of the dimerization pair is linked intracellularly to the transmembrane domain.

7. The conditionally repressible synthetic ICR of claim 5, wherein the second member of the dimerization pair is extracellular and linked to the intracellular inhibitory domain by way of the transmembrane domain.

8. The conditionally repressible synthetic ICR of any one of the preceding claims, wherein the stimulatory ICR binds a soluble antigen.

9. The conditionally repressible synthetic ICR of any one of the preceding claims, wherein the stimulatory ICR binds a cell surface antigen.

10. The conditionally repressible synthetic ICR of any one of the preceding claims, wherein the stimulatory ICR binds a protein associated with the TCR complex.

11. The conditionally repressible synthetic ICR of any one of the preceding claims, wherein the intracellular inhibitory domain is an inhibitory domain derived from a protein selected from the group consisting of: PD-1, CTLA4, HPK1, SHP1, SHP2, Sts1 and Csk.

12. The conditionally repressible synthetic ICR of any one of the preceding claims, wherein the synthetic stimulatory ICR comprises an intracellular signaling domain selected from the group consisting of: a CD3-zeta signaling domain, a ZAP70 signaling domain and an immunoreceptor tyrosine-based activation motif (ITAM).

13. The conditionally repressible synthetic ICR of any one of the preceding claims, wherein the synthetic stimulatory ICR comprises an intracellular signaling domain that comprises a lymphocyte-specific protein tyrosine kinase (Lck) interaction sites.

14. The conditionally repressible synthetic ICR of any one of the preceding claims, wherein the first and second members of the dimerization pair form a homodimer in the presence of a small molecule dimerizer.

15. The conditionally repressible synthetic ICR of any one of the preceding claims, wherein the first and second members of the dimerization pair form a heterodimer in the presence of a small molecule dimerizer.

16. The conditionally repressible synthetic ICR of any one of the preceding claims, wherein the dimerization pair is a dimerization pair responsive to a small molecule selected from the group consisting of: rapamycin or an analog thereof, gibberellic acid or an analog thereof, coumermycin or an analog thereof, methotrexate or an analog thereof, abscisic acid or an analog thereof and tamoxifen or an analog thereof.

17. The conditionally repressible synthetic ICR of any one of the preceding claims, wherein the synthetic stimulatory ICR is a synthetic chimeric antigen receptor (CAR) or portion thereof.

18. The conditionally repressible synthetic ICR of any one of the preceding claims, wherein the synthetic stimulatory ICR is a synthetic T cell receptor (TCR) or portion thereof.

19. The conditionally repressible synthetic ICR of any one of the preceding claims, wherein the synthetic stimulatory ICR is a T cell-antigen coupler (TAC) or portion thereof.

20. A mammalian cell genetically modified to produce the heteromeric, conditionally repressible synthetic ICR of any of the preceding claims.

21. The cell of claim 20, wherein the cell is an immune cell.

22. The cell of claim 21, wherein the immune cell is a T cell.

23. The cell of claim 22, wherein the T cell is a CD4 T cell.

24. The cell of claim 22, wherein the T cell is a CD8 T cell.

25. A nucleic acid comprising a nucleotide sequence encoding the heteromeric, conditionally repressible synthetic ICR of any one of claims 1 to 19.

26. The nucleic acid of claim 25, wherein the nucleotide sequence is operably linked to a T cell specific promoter or a regulatable promoter.

27. A recombinant expression vector comprising the nucleic acid of claim 25 or 26.

28. The nucleic acid of claim 25, wherein the nucleic acid is in vitro transcribed RNA.

29. A method of repressing T cell activation, the method comprising:
contacting a T cell that expresses a heteromeric, conditionally repressible synthetic ICR of any one of claims 1-19 and has been activated by binding of an antigen or epitope to the synthetic stimulatory ICR with a dimerizing agent; wherein, in the presence of the dimerizing agent, the first and second members of the dimerization pair dimerize and the intracellular inhibitory domain represses the activation of the T cell.

30. The method of claim 29, wherein said contacting occurs in vivo.

31. A method of making the cell of any of claims 20 to 24, the method comprising genetically modifying a mammalian cell with an expression vector comprising nucleotide sequences encoding the conditionally repressible synthetic ICR of any one of claims 1 to 19, or genetically modifying a mammalian cell with an RNA comprising nucleotide sequences encoding the conditionally repressible synthetic ICR of any one of claims 1 to 19.

32. The method of claim 31, wherein said genetic modification is carried out ex vivo.

33. The method of claim 31 or 32, wherein the cell is a T lymphocyte, a stem cell, an NK cell, a progenitor cell, a cell derived from a stem cell, or a cell derived from a progenitor cell.

34. A method of modulating treatment of a cancer in an individual, the method comprising:
genetically modifying an immune cell or immune cell progenitor obtained from the individual with an expression vector comprising nucleotide sequences encoding the conditionally repressible synthetic ICR of any one of claims 1 to 19, wherein the synthetic stimulatory ICR is specific for an epitope on a cancer cell in the individual;
treating the individual with the genetically modified immune cell, immune cell progenitor or progeny thereof under conditions sufficient for killing of the cancer cell; and
modulating the treatment of the individual by administering to the individual an effective amount of a dimerizing agent, wherein the dimerizing agent induces dimerization of the first and second members of the dimerization pair, wherein said dimerization provides for repression of the genetically modified immune cell, immune cell progenitor or progeny thereof.

35. The method of claim 34, wherein the genetic modification is carried out ex vivo and the treating comprises introducing the genetically modified immune cell, immune cell progenitor or progeny thereof into the individual.

36. A method of repressing the activity of a host cell, the method comprising contacting an activated host cell with a dimerizing agent, wherein the host cell is genetically modified to produce a conditionally repressible synthetic ICR of any one of claims 1 to 19, and wherein, in the presence of the dimerizing agent the first and second dimerizing members of the conditionally repressible synthetic ICR dimerize and represses at least one activity of the activated host cell.

37. The method of claim 36, wherein the activity is selected from the group consisting of: proliferation, cell survival, apoptosis, gene expression, immune activation and combinations thereof.

38. A heteromeric, conditionally repressible synthetic chimeric antigen receptor (CAR) comprising:
a synthetic stimulatory CAR comprising:
i) a extracellular recognition domain;
ii) a transmembrane domain linked to the extracellular recognition domain;
iii) a first member of a dimerization pair linked to the transmembrane domain; and
iv) an intracellular stimulation domain; and
a synthetic CAR repressor comprising:
i) a second member of the dimerization pair; and
ii) an intracellular inhibitory domain linked to the second member of the dimerization pair.

39. The heteromeric, conditionally repressible synthetic CAR of claim 38, wherein the synthetic CAR repressor further comprises a transmembrane domain linked to the second member of the dimerization pair, the intracellular inhibitory domain or both.

40. A heteromeric, conditionally repressible synthetic T cell receptor (TCR) comprising:
a synthetic stimulatory TCR comprising:
i) a transmembrane domain;
ii) a first member of a dimerization pair linked to the transmembrane domain;
iii) an engineered TCR polypeptide comprising at least one TCR alpha or beta chain, wherein the at least one TCR alpha or beta chain is linked to the transmembrane domain or the first member of a dimerization pair; and
a synthetic TCR repressor comprising:
i) a second member of the dimerization pair; and
ii) an intracellular inhibitory domain linked to the second member of the dimerization pair.

41. The heteromeric, conditionally repressible synthetic TCR of claim 40, wherein the synthetic TCR repressor further comprises a transmembrane domain linked to the second member of the dimerization pair, the intracellular inhibitory domain or both.

42. The heteromeric, conditionally repressible synthetic TCR of claim 40 or 41, wherein the engineered TCR polypeptide further comprises a TCR gamma chain.

43. A heteromeric, conditionally repressible T cell-antigen coupler (TAC) comprising:
a synthetic stimulatory TAC comprising:
i) a TCR specific binding domain;
ii) a transmembrane domain;
iii) a intracellular signaling domain; and
iv) a first member of a dimerization pair; and
a synthetic TAC repressor comprising:
i) a second member of the dimerization pair; and
ii) an intracellular inhibitory domain linked to the second member of the dimerization pair.

44. The heteromeric, conditionally repressible TAC of claim 43, wherein the synthetic stimulatory TAC further comprises a target-specific binding domain.

45. The heteromeric, conditionally repressible TAC of claim 43 or 44, wherein the synthetic TAC repressor further comprises a transmembrane domain linked to the second member of the dimerization pair, the intracellular inhibitory domain or both.

46. The heteromeric, conditionally repressible TAC of any of claims 43-45, wherein the TCR specific binding domain specifically binds CD3.
